(19) United States
(12) Patent Application Publication
(10) Pub. No.: US 2020/0109408 A1
(43) Pub. Date: Apr. 9, 2020

(54) METHODS OF SELECTING CELLS COMPRISING GENOME EDITING EVENTS

(71) Applicant: Tropic Biosciences UK Limited, Norwich (GB)

(72) Inventors: Eyal MAORI, Cambridge (GB); Yaron GALANTY, Cambridge (GB); Cristina PIGNOCCHI, Norwich (GB); Angela CHAPARRO GARCIA, Norwich (GB); Ofir MEIR, Norwich (GB)

(73) Assignee: Tropic Biosciences UK Limited, Norwich (GB)

(21) Appl. No.: 16/617,515

(22) PCT Filed: May 31, 2018
(86) PCT No.: PCT/IB2018/053905

§ 371 Date: Nov. 27, 2019

(30) Foreign Application Priority Data
May 31, 2017 (GB) 1708661.2
May 31, 2017 (GB) 1708664.6
May 31, 2017 (GB) 1708666.1

(51) Int. Cl. C12N 15/82; C12N 15/10; C12N 9/22

(52) U.S. Cl. CPC C12N 15/8209; C12N 15/102; C12N 2800/80; C12N 2310/20; C12N 9/22

(57) ABSTRACT

Nucleic acid constructs for use in a method of selecting cells comprising a genome editing event, the method comprising (a) transforming cells of a plant of interest with the nucleic acid construct; (b) selecting transformed cells exhibiting fluorescence emitted by the fluorescent reporter using flow cytometry or imaging; and (c) culturing the transformed cells comprising the genome editing event by the DNA editing agent for a time sufficient to lose expression of the DNA editing agent so as to obtain cells which comprise a genome editing event generated by the DNA editing agent but lack DNA encoding the DNA editing agent.

### FIELD AND BACKGROUND OF THE INVENTION

The present invention, in some embodiments thereof, relates to methods of selecting cells comprising genome editing events.

To meet the challenge of increasing global demand for food production, the typical approaches to improving agricultural productivity (e.g. enhanced yield or engineered pest resistance) have relied on either mutation breeding or introduction of novel genes into the genomes of crop species by transformation. These processes are inherently nonspecific and relatively inefficient. For example, plant transformation methods deliver exogenous DNA that integrates into the genome at random locations. Thus, in order to identify and isolate transgenic plant lines with desirable attributes, it is necessary to generate hundreds of unique random integration events per construct and subsequently screen for the desired individuals. As a result, conventional plant trait engineering is a laborious, time-consuming, and unpredictable undertaking. Furthermore, the random nature of these integrations makes it difficult to predict whether pleiotropic effects due to unintended genome disruption have occurred.

The random nature of the current transformation processes requires the generation of hundreds of events for the identification and selection of transgene event candidates (transformation and event screening is rate limiting relative to gene candidates identified from functional genomic studies). In addition, depending upon the location of integration within the genome, a gene expression cassette may be expressed at different levels as a result of the genomic position effect. As a result, the generation, isolation and characterization of plant lines with engineered genes or traits has been an extremely labor and cost-intensive process with a low probability of success. In addition to the hurdles associated with selection of transgenic events, some major concerns related to gene confinement and the degree of stringency required for release of a transgenic plants into the environment for commercial applications arise.

Recent advances in genome editing techniques have made it possible to alter DNA sequences in living cells. Genome editing is more precise than conventional crop breeding methods or standard genetic engineering (transgenic or GM) methods. By editing only a few of the billions of nucleotides (the building blocks of genes) in the cells of plants, these new techniques might be the most effective way to get crops to grow better in harsh climates, resist pests or improve nutrition. Because the more precise the technique, the less of the genetic material is altered, so the lower the uncertainty about other effects on how the plant behaves.

The most established method of plant genetic engineering using CRISPR Cas9 genome editing technology requires the insertion of new DNA into the host's genome. This insert (e.g., a transfer DNA (T-DNA) based construct) carries several transcriptional units in order to achieve successful CRISPR Cas9 genome edits. These commonly consist of an antibiotic resistance gene to select for transgenic plants, the Cas9 machinery, and several sgRNA units. Because of the integration of foreign DNA into the genome, plants generated this way are classified as transgenic or genetically modified (GM). Once a genome edit has been established in the host, this T-DNA backbone can be removed through sexual propagation and breeding, as the CRISPR Cas9 machinery is no longer needed to maintain the phenotype. However, commercial crops like cultivated banana, pineapple and fig species are parthenocarpic (do not produce viable seeds) rendering the removal of T-DNA backbone by sexual reproduction impossible.

Additional background art includes:
U.S. Patent Application 20140075593;
Zhang, Y., et al., Efficient and transgene-free genome editing in wheat through transient expression of CRISPR/Cas9 DNA or RNA. Nat Commun, 2016. 7: p. 12617;
Woo, J. W., et al., DNA-free genome editing in plants with preassembled CRISPR-Cas9 ribonucleoproteins. Nat Biotechnol, 2015. 33(11): p. 1162-4;
Svitashev, S., et al., Genome editing in maize directed by CRISPR-Cas9 ribonucleoprotein complexes. Nat Commun, 2016. 7: p. 13274;
Luo, S., et al., Non-transgenic Plant Genome Editing Using Purified Sequence-Specific Nucleases. Mol Plant, 2015. 8(9): p. 1425-7;
Hoffmann 2017 PlosOne 12(2):e0172630; and
Chiang et al., 2016. SP1,2,3. Sci Rep. 2016 Apr. 15; 6:24356. doi: 10.1038/srep24356.

### SUMMARY OF THE INVENTION

According to an aspect of some embodiments of the present invention there is provided a nucleic acid construct comprising:

(i) a nucleic acid sequence encoding a genome editing agent;
(ii) a nucleic acid sequence encoding a fluorescent reporter,
the nucleic acid sequence encoding the genome editing agent and the nucleic acid sequence encoding the fluorescent reporter being operatively linked to a plant promoter.

According to some embodiments of the invention, each of the nucleic acid sequence encoding the genome editing agent and the nucleic acid sequence encoding the fluorescent reporter being operatively linked to a terminator.

According to some embodiments of the invention, the genome editing agent comprises an endonuclease.

According to some embodiments of the invention, the genome editing agent is of a DNA editing system selected from the group consisting of a meganuclease, a zinc finger nucleases (ZFN), a transcription-activator like effector nuclease (TALEN) and CRISPR.

According to some embodiments of the invention, the endonuclease comprises Cas-9.

According to some embodiments of the invention, the genome editing agent comprises a nucleic acid agent encoding at least one gRNA operatively linked to a plant promoter.

According to some embodiments of the invention, the fluorescent reporter is detectable by fluorescent activated cell sorter (FACS).

According to some embodiments of the invention, the fluorescent reporter is a green fluorescent protein (GFP) or a GFP derivative.

According to some embodiments of the invention, the plant promoters are identical.

According to some embodiments of the invention, the plant promoters are different.

According to some embodiments of the invention, the promoters comprise a 35S promoter.

According to some embodiments of the invention, the promoters comprise a U6 promoter.

According to some embodiments of the invention, the promoters comprise a U6 promoter operatively linked to the nucleic acid agent encoding at least one gRNA and a 35S promoter operatively linked to the nucleic acid sequence encoding the genome editing agent or the nucleic acid sequence encoding the fluorescent reporter.

According to an aspect of some embodiments of the present invention there is provided a cell comprising the nucleic acid construct as described herein.

According to some embodiments of the invention, the cell is a plant cell.

According to some embodiments of the invention, the plant cell is a protoplast.

According to an aspect of some embodiments of the present invention there is provided a method of selecting cells comprising a genome editing event, the method comprising:

(a) transforming cells of a plant of interest with the nucleic acid construct as described herein;

(b) selecting transformed cells exhibiting fluorescence emitted by the fluorescent reporter using flow cytometry or imaging; and

(c) culturing the transformed cells comprising the genome editing event by the DNA editing agent for a time sufficient to lose expression of the DNA editing agent so as to obtain cells which comprise a genome editing event generated by the DNA editing agent but lack DNA encoding the DNA editing agent.

According to some embodiments of the invention, the method further comprises validating in the transformed cells loss of expression of the fluorescent reporter following step (c).

According to some embodiments of the invention, the method further comprises validating in the transformed cells loss of expression of the DNA editing agent following step (c).

According to some embodiments of the invention, the validating is by imaging.

According to some embodiments of the invention, the validating comprises sequencing.

According to some embodiments of the invention, the validating comprises a structure-selective enzyme that recognizes and cleaves mismatched DNA.

According to some embodiments of the invention, the enzyme comprises a T7 endonuclease.

According to some embodiments of the invention, step (b) is effected 24-72 hours following step (a).

According to some embodiments of the invention, step (c) is effected for at least −60-100 days.

According to some embodiments of the invention, step (c) is effected in the absence of an effective amount of antibiotics.

According to some embodiments of the invention, the cells comprise protoplasts.

According to some embodiments of the invention, the method further comprises regenerating plants following steps (c) from the transformed cells which comprise the genome editing event but lack the DNA encoding the DNA editing agent.

Yet another aspect of the disclosure includes methods of editing the genome of one or more cells without integration of a selectable marker or screenable reporter into the genome comprising:

(a) transforming one or more cells of a plant of interest with a nucleic acid construct comprising:

(i) a nucleic acid sequence encoding a genome editing agent;

(ii) a nucleic acid sequence encoding a fluorescent reporter,

the nucleic acid sequence encoding said genome editing agent and the nucleic acid sequence encoding the fluorescent reporter being operatively linked to a plant promoter;

(b) selecting transformed cells exhibiting fluorescence emitted by said fluorescent reporter using flow cytometry or imaging; and

(c) culturing said transformed cells comprising a genome editing event generated by the genome editing agent for a time sufficient to lose the nucleic acid construct so as to obtain cells which comprise the genome editing event generated by the genome editing agent but lack the nucleic acid construct and the nucleic acid sequence encoding the genome editing agent.

According to some embodiments of this aspect the nucleic acid construct is non-integrating.

According to some embodiments of this aspect, which may be combined with the preceding embodiment, the nucleic acid sequence encoding the fluorescent reporter is non-integrating.

According to a further embodiment of the preceding embodiment, the non-integrating nucleic acid sequence encoding the fluorescent reporter lack flanking sequences homologous to the genome of the plant of interest.

According to some embodiments of this aspect, which may be combined with any of the preceding embodiments, the genome editing event comprises a deletion, a single base pair substitution, or an insertion of genetic material from a second plant that could otherwise be introduced into the plant of interest by traditional breeding.

According to some embodiments of this aspect, which may be combined with any of the preceding embodiments, the genome editing event does not comprise the introduction of foreign DNA into the genome of the plant of interest that could not be introduced through traditional breeding.

According to some embodiments of this aspect, which may be combined with any of the preceding embodiments, each of the nucleic acid sequence encoding the genome editing agent and the nucleic acid sequence encoding the fluorescent reporter being operatively linked to a terminator.

According to some embodiments of this aspect, which may be combined with any of the preceding embodiments, the genome editing agent comprises an endonuclease.

According to some embodiments of this aspect, which may be combined with any of the preceding embodiments, the genome editing agent is a DNA editing system selected from the group consisting of a meganuclease, a zinc finger nucleases (ZFN), a transcription-activator like effector nuclease (TALEN) and CRISPR.

According to some embodiments of this aspect, which include endonucleases, the endonuclease comprises Cas-9.

According to some embodiments of this aspect, which may be combined with any of the preceding embodiments, the genome editing agent comprises a nucleic acid agent encoding at least one gRNA operatively linked to a plant promoter.

According to some embodiments of this aspect, which may be combined with any of the preceding embodiments, the fluorescent reporter is detectable by fluorescent activated cell sorter (FACS).

According to some embodiments of this aspect, which may be combined with any of the preceding embodiments, the fluorescent reporter is a green fluorescent protein (GFP) or a GFP derivative.

According to some embodiments of this aspect, which may be combined with any of the preceding embodiments, the plant promoters are identical.

According to some embodiments of this aspect, which may be combined with any of the preceding embodiments, the plant promoters are different.

According to some embodiments of this aspect, which may be combined with any of the preceding embodiments, at least one of the promoters comprises a 35S promoter.

According to some embodiments of this aspect, which may be combined with any of the preceding embodiments, at least one of the promoters comprises a U6 promoter.

According to some embodiments of this aspect, which may be combined with any of the preceding embodiments, the plant promoter operatively linked to the nucleic acid agent encoding at least one gRNA is a U6 promoter and the plant promoter operatively linked to the nucleic acid sequence encoding said genome editing agent or to the nucleic acid sequence encoding said fluorescent reporter is a CaMV 35S promoter.

According to some embodiments of this aspect, which may be combined with any of the preceding embodiments, further validating the transformed cells loss of the nucleic acid sequence encoding a fluorescent reporter following step (c) is performed.

According to some embodiments of this aspect, which may be combined with any of the preceding embodiments, further validating in said transformed cells loss of the nucleic acid sequence encoding the genome editing agent following step (c) is performed.

According to some embodiments of this aspect, which include further validating, the further validating is by imaging.

According to some embodiments of this aspect, which include further validating, the further validating comprises sequencing.

According to some embodiments of this aspect, which include further validating, the further validating comprises a structure-selective enzyme that recognizes and cleaves mismatched DNA.

According to some embodiments of this aspect, which include a structure-selective enzyme, the structure-selective enzyme comprises a T7 endonuclease.

According to some embodiments of this aspect, which may be combined with any of the preceding embodiments, step (b) is effected 24-72 hours following step (a).

According to some embodiments of this aspect, which may be combined with any of the preceding embodiments, step (c) is effected for at least 60-100 days.

According to some embodiments of this aspect, which may be combined with any of the preceding embodiments, step (c) is effected in the absence of an effective amount of antibiotics.

According to some embodiments of this aspect, which may be combined with any of the preceding embodiments, said cells comprise protoplasts.

According to some embodiments of this aspect, which may be combined with any of the preceding embodiments, further regenerating plants following steps (c) from said transformed cells which comprise said genome editing event but lack said DNA encoding said DNA editing agent is performed.

Still another aspect of the disclosure includes nucleic acid construct for editing the genome of one or more plant cells without integration of a selectable marker or screenable reporter comprising:

(i) a nucleic acid sequence encoding a genome editing agent;

(ii) a nucleic acid sequence encoding a fluorescent reporter,

said nucleic acid sequence encoding said genome editing agent and said nucleic acid sequence encoding said fluorescent reporter being operatively linked to a plant promoter.

According to some embodiments of this aspect the nucleic acid construct is non-integrating.

According to some embodiments of this aspect, which may be combined with the preceding embodiment, the nucleic acid sequence encoding a fluorescent reporter is non-integrating.

According to a further embodiment of the preceding embodiment, the non-integrating nucleic acid sequence encoding the fluorescent reporter lack flanking sequences homologous to the genome of the plant of interest.

According to some embodiments of this aspect, which may be combined with any of the preceding embodiments, the genome editing event comprises a deletion, a single base pair substitution, or an insertion of genetic material from a second plant that could otherwise be introduced into the plant of interest by traditional breeding.

According to some embodiments of this aspect, which may be combined with any of the preceding embodiments, the genome editing event does not comprise the introduction of foreign DNA into the genome of the plant of interest that could not be introduced through traditional breeding.

According to some embodiments of this aspect, which may be combined with any of the preceding embodiments, each of the nucleic acid sequence encoding the genome editing agent and the nucleic acid sequence encoding the fluorescent reporter being operatively linked to a terminator.

According to some embodiments of this aspect, which may be combined with any of the preceding embodiments, the genome editing agent comprises an endonuclease.

According to some embodiments of this aspect, which may be combined with any of the preceding embodiments, the genome editing agent is a DNA editing system selected from the group consisting of a meganuclease, a zinc finger nucleases (ZFN), a transcription-activator like effector nuclease (TALEN) and CRISPR.

According to some embodiments of this aspect, which include an endonuclease, the endonuclease comprises Cas-9.

According to some embodiments of this aspect, which may be combined with any of the preceding embodiments, the genome editing agent comprises a nucleic acid agent encoding at least one gRNA operatively linked to a plant promoter.

According to some embodiments of this aspect, which may be combined with any of the preceding embodiments, the fluorescent reporter is detectable by fluorescent activated cell sorter (FACS).

According to some embodiments of this aspect, which may be combined with any of the preceding embodiments, the fluorescent reporter is a green fluorescent protein (GFP) or a GFP derivative.

According to some embodiments of this aspect, which may be combined with any of the preceding embodiments, the plant promoters are identical.

According to some embodiments of this aspect, which may be combined with any of the preceding embodiments, the plant promoters are different.

According to some embodiments of this aspect, which may be combined with any of the preceding embodiments, at least one of the promoters comprises a 35S promoter.

According to some embodiments of this aspect, which may be combined with any of the preceding embodiments, at least one of the promoters comprises a U6 promoter.

According to some embodiments of this aspect, which may be combined with any of the preceding embodiments, the plant promoter operatively linked to the nucleic acid agent encoding at least one gRNA is a U6 promoter and the plant promoter operatively linked to the nucleic acid sequence encoding said genome editing agent or to the nucleic acid sequence encoding said fluorescent reporter is a CaMV 35S promoter.

Another aspect still includes cells comprising the nucleic acid construct the preceding aspect and any and all embodiments and combinations of embodiments.

According to some embodiments of this aspect, the cell is a plant cell.

According to some embodiments of the preceding embodiment, the plant cell is a protoplast.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an embodiment of the method of selecting cells comprising a genome editing event;

FIGS. 2A-B show positive transfection of banana and coffee protoplasts with mCherry or GFP plasmids respectively. 1×10⁶ banana and coffee protoplasts were transfected using PEG with plasmid (pAC2010) carrying mCherry (fluorescent marker) (FIG. 2A) or pDK1202 carrying GFP (fluorescent marker) (FIG. 2B). 3 days post-transfection, the transfection efficiency was analysed under a fluorescent microscope. FIG. 2A. Banana protoplasts, upper panel brightfield, lower panel fluorescence; FIG. 2B. Coffee protoplasts, upper panel brightfield, lower panel fluorescence.

FIGS. 3A-B show FACS enrichment of positive mCherry banana and dsRed coffee protoplasts. 1×10⁶ banana (FIG. 3A) and coffee (FIG. 3B) protoplasts were transfected using PEG with plasmid pAC2010 (FIG. 3A, right panel) or pDK2023 (FIG. 3B, right panel) carrying the fluorescent marker mCherry (FIG. 3A) or dsRed (FIG. 3B). Three (FIG. 3A) or 4 (FIG. 3B) days post-transfection protoplasts were analyzed by FACS, all positive cells were sorted and collected. FIG. 3A. FACS analysis of banana protoplasts-enrichment and collection of positive mCherry expressing protoplasts. FIG. 3B. FACS analysis of coffee protoplasts-enrichment and collection of positive dsRed expressing protoplasts FIG. 3C shows FACS enrichment of positive mCherry banana protoplasts. Enrichment of mCherry banana protoplasts was confirmed by fluorescent microscopy. Unsorted (upper panels) and sorted (lower panels) transfected protoplasts were imaged with a fluorescent microscope at 3 days post transfection.

FIGS. 4A-B show the quantification of genome editing activity in tobacco (FIG. 4A) and coffee (FIG. 4B) using FACS. Protoplasts were transfected with different versions of the sensor construct (1 to 4) each expressing GFP+mCherry and different sgRNAs against GFP. Positive editing of the GFP marker was evaluated by measuring the reduction of the GFP signal compared to the control without sgRNA. Three (FIG. 4A) or 4 (FIG. 4B) days after transfection, cells were analysed for efficient genome editing and the ratio of green versus red protoplasts was measured. The efficiency of the sensor was measured by the reduction of the green/red protoplasts ratio. All sensor constructs with specific sgRNA showed a reduction of green versus red when compared to the control plasmid in both tobacco and coffee. Sensor 1 to 4 refers to 4 different plasmids that have different sgRNAs under different U6 promoters targetting GFP. Sensor 1: pU6+sgRNA-eGFP1; sensor 2 pU6+sgRNA-eGFP2; Sensor 3: pU6-26+sgRNA-eGFP1; sensor 4 pU6-26+sgRNA-eGFP2.

FIGS. 5A-C show the decrease of mCherry positive banana protoplasts over time indicating transient transformation events. Banana protoplasts transfected with a plasmid carrying the mCherry fluorescent marker were imaged at 3 (FIG. 5A) and 10 (FIG. 5B) days post transfection. FIG. 5C. Progressive reduction in number of mCherry positive protoplasts up to 25 days post transfection, measured by FACS. 100% represents the proportion of cherry-expressing cells at 3 days post-transfection.

FIG. 6A shows the decrease of mCherry-positive banana protoplasts over time indicating transient transformation events. Non-sorted protoplasts imaged before FACS. Musa acuminata protoplasts were transfected with a plasmid carrying the mCherry fluorescent marker (pAC2010) or with no DNA. Non-sorted protoplasts were imaged at 3, 6, and 10 days post transfection as indicated. Microscopy images show the progressive reduction in number and intensity of mCherry-positive protoplasts along time. BF (Bright field).

FIG. 6B shows the decrease of mCherry-positive protoplasts over time indicating transient transformation events. Sorted protoplasts and imaged after FACS. Musa acuminata protoplasts transfected with a plasmid carrying the mCherry fluorescent marker (2010) were sorted and imaged at 3, 6, and 10 days post transfection as indicated. Microscopy images show the progressive reduction in number and intensity of mCherry-positive protoplasts along time. BF (Bright field).

FIGS. 7A-B show identification and targeting of the coffee PDS gene Cc04_g00540. (A) is a cartoon illustrating the major features of the gene: yellow boxes represent exons, numbers 110 and 113 above horizontal arrows show the primers used for amplification of the target area, and the positions of the sgRNAs 1 to 4 are indicated. (B) Cc04_g00540 was amplified flanking sgRNA1 to 4 regions (panel A) using DNA extracted at 6 days post transfection from coffee transfected and sorted protoplasts as template. Samples were transfected with the following plasmids: (1) pDK2028 (sgRNA 165+sgRNA166 targeting Cc04_g00540), (2) pDK2029 (sgRNA167+sgRNA168 targeting Cc04_g00540) as depicted in A, (3) pDK2030 (as a control, sgRNA targeting an unrelated gene) and (4) PCR negative control (no DNA). The agarose gel shows that treatment with plasmid pDK2029 induces indels as reflected by the additional bands in sample 2, which are not observed in the other samples.

FIGS. 8A-C show identification and targeting of the banana PDS gene Ma08_g1 6510. (A) is a cartoon representing the Ma08_g16510 locus indicating the relative positions where the sgRNAs were designed and the primers used for further analysis. (FIG. 8B) DNA extracted at 6 days post transfection from banana transfected and sorted protoplasts was used as template to amplify the Ma08_g16510 locus with specific primers outside of the sgRNAs region as indicated in panel A. Samples were transfected with the following plasmids: (P2) pAC2023 (sgRNA227+sgRNA224 targeting Ma08_g16510), (P4) pAC2024 (sgRNA228+sgRNA224 targeting Ma08_g16510), (ctr) pAC2010 (as a control, no sgRNA), (−) PCR negative control (no DNA) and (WT) is wildtype M. acuminata gDNA. The agarose gel shows that treatment with plasmid pAC2023 induces a clear deletion as reflected by the additional band in sample P2, which are not observed in the other samples. (FIG. 8C) is the alignment of the sequenced amplicons of WT and P2 samples showing the deletion seen in FIG. 8B.

### DESCRIPTION OF SPECIFIC EMBODIMENTS OF THE INVENTION

The present invention, in some embodiments thereof, relates to methods of selecting cells comprising genome editing events.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The most established method of plant genetic engineering using CRISPR-Cas genome editing technology requires the insertion of new DNA into the host's genome. This insert, a transfer DNA (T-DNA), carries several transcriptional units in order to achieve successful CRISPR-Cas-mediated genome edits. These commonly consist of an antibiotic resistance gene to select for transgenic plants, the Cas machinery, and several sgRNA units. Because of the integration of foreign DNA into the genome, plants generated this way are classified as transgenic or genetically modified (GM). Once a genome edit has been established in the host, the T-DNA can be removed through sexual propagation and breeding, as the CRISPR Cas9 machinery is no longer needed to maintain the phenotype. However, for parthenocarpic crops that do not produce viable seeds, removal of T-DNA by sexual reproduction is impossible.

Whilst reducing embodiments of the invention to practice, the present inventors devised a novel selection method which can be used to elicit genome editing events without carrying a transgene in the final product, even in parthenocarpic crops.

Specifically, embodiments of the invention rely on the transient transfection of a nucleic acid construct comprising a genome editing module/agent and a reporter gene. Shortly after transfection, transformants are positively selected based on expression of the reporter gene (e.g., using flow cytometry) and sequencing to identify cells exhibiting an editing event. These cells are then cultured in the absence of antibiotics so as to allow losing expression of the reporter gene and the DNA editing agent. A non-transgenic genome editing event is confirmed at the level of expression e.g., cytometry/imaging (to affirm the absence of the reporter gene) and/or at the DNA sequence level.

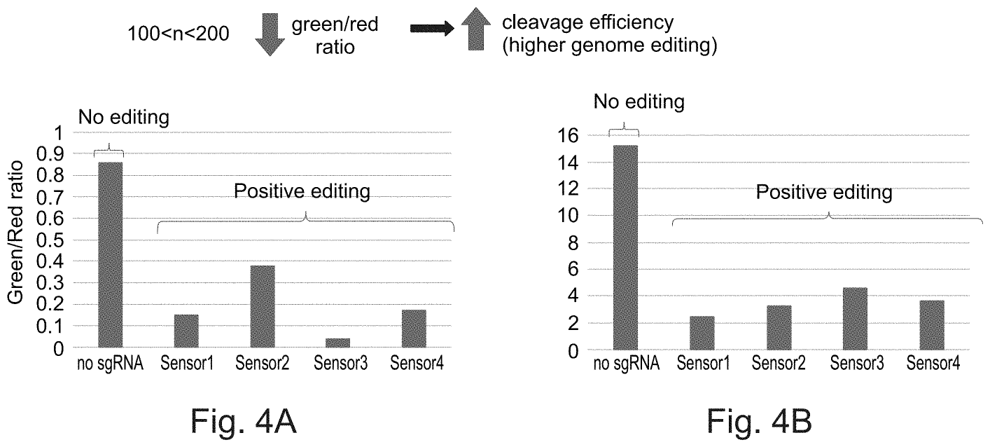

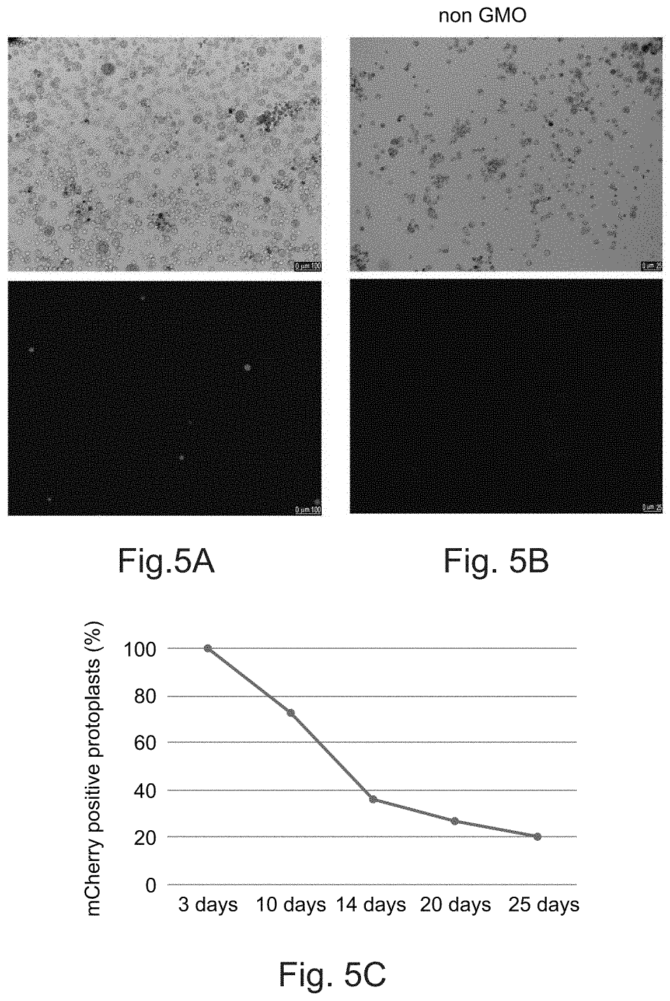

As is illustrated herein and in the Examples section which follows, the present inventors were able to transform banana, coffee and tobacco protoplasts. The transformed cells expressed a fluorescent target gene (e.g., GFP) and a reporter gene (e.g., mCherry, dsRed) having distinct fluorescent signals than the target gene along with a genome editing agent directed to the target gene. The present inventors were able to efficiently edit the target as evidenced by FIG. 4 while avoiding stable transgenesis, as evidenced by FIGS. 5A-C to 6A-B.

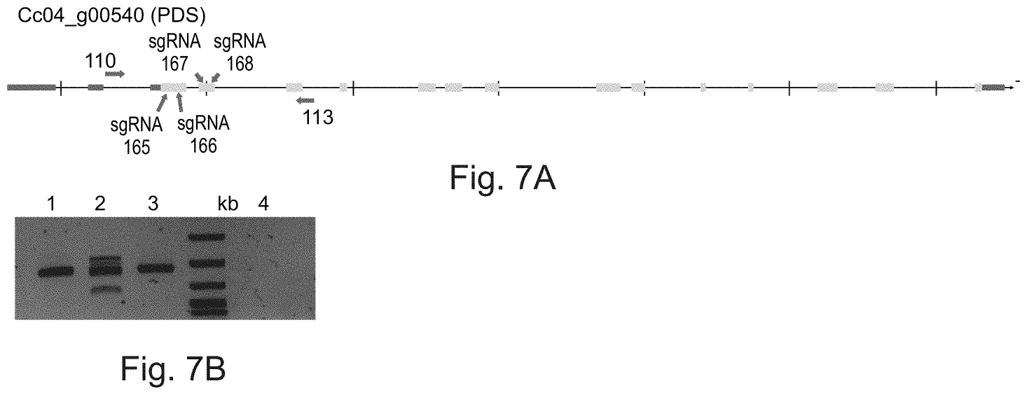

The present inventors also used the selection system of some embodiments of the invention for effectively enriching genome editing events on an endogenous gene, e.g., PDS, as shown in FIGS. 7A-B and 8A-C, without stable transgenesis.

Hence the present methodology allows genome editing without integration of a selectable or screenable reporter.

Non-transgenic cells selected using this method can be regenerated to plants in a simple and economical manner even for non-parthenocarpic plants, negating the need for crossing and back-crossing thus rendering the process cost- and time-effective.

Thus, according to an aspect of the invention there is provided a nucleic acid construct comprising:

(i) a nucleic acid sequence encoding a genome editing agent;
(ii) a nucleic acid sequence encoding a fluorescent reporter,

the nucleic acid sequence encoding the genome editing agent and the nucleic acid sequence encoding the fluorescent reporter each being operatively linked to a plant promoter.

Following is a description of various non-limiting examples of methods and DNA editing agents used to introduce nucleic acid alterations to a nucleic acid sequence (genomic) of interest and agents for implementing same that can be used according to specific embodiments of the present disclosure.

According to a specific embodiment, the genome editing agent comprises an endonuclease, which may comprise or have an auxiliary unit of a DNA targeting module.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJF). NHEJF directly joins the DNA ends in a double-stranded break, while HDR utilizes a homologous donor sequence as a template for regenerating the missing DNA sequence at the break point. In order to introduce specific nucleotide modifications to the genomic DNA, a donor DNA repair template containing the desired sequence must be present during HDR.

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved motif after which they are named. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered in a manner such that these nucleases can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALENs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALENs on the other hand are found in repeats with a one-to-one recognition ratio between the amino acids and the recognized nucleotide pairs. Because both zinc fingers and TALENs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system (also referred to herein as “CRISPR”) Many bacteria and archaea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system is coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present disclosure include those described in the Example section which follows.

In order to use the CRISPR system, both gRNA and a CAS endonuclease (e.g. Cas9) should be expressed in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A—Cambridge, Mass. 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. CAS endonucleases that can be used to effect DNA editing with gRNA include, but are not limited to, Cas9, Cpf1 (Zetsche et al., 2015, Cell. 163(3):759-71), C2c1, C2c2, and C2c3 (Shmakov et al., Mol Cell. 2015 Nov. 5; 60(3):385-97).

According to a specific embodiment, the CRISPR comprises a sgRNA comprising a nucleic acid sequence selected from the group consisting of SEQ ID NOs: 10-33.

As mentioned, the nucleic acid construct comprises a nucleic acid agent encoding a fluorescent protein.

As used herein, “a fluorescent protein” refers to a polypeptide that emits fluorescence and is typically detectable by flow cytometry or imaging, therefore can be used as a basis for selection of cells expressing such a protein.

Examples of fluorescent proteins that can be used as reporters are the Green Fluorescemt Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

GFP is a protein composed of 238 amino acid residues (26.9 kDa) that exhibits bright green fluorescence when exposed to light in the blue to ultraviolet range. Although many other marine organisms have similar green fluorescent proteins, GFP traditionally refers to the protein first isolated from the jellyfish Aequorea victoria. The GFP from A. victoria has a major excitation peak at a wavelength of 395 nm and a minor one at 475 nm. Its emission peak is at 509 nm, which is in the lower green portion of the visible spectrum. The fluorescence quantum yield (QY) of GFP is 0.79. The GFP from the sea pansy (Renilla reniformis) has a single major excitation peak at 498 nm. GFP makes for an excellent tool in many areas of biology due to its ability to form internal chromophores without requiring any accessory cofactors, gene products, or enzymes/substrates other than molecular oxygen.

Also contemplated are GFP derivatives e.g., S65T mutation that dramatically improves the spectral characteristics of GFP, resulting in increased fluorescence, photostability, and a shift of the major excitation peak to 488 nm, with the peak emission kept at 509 nm. This matches the spectral characteristics of commonly available FITC filter sets. The F64L point mutant yields enhanced GFP (EGFP). EGFP has an extinction coefficient (denoted ε) of 55,000 M⁻¹cm⁻¹. The fluorescence quantum yield (QY) of EGFP is 0.60. The relative brightness, expressed as ε·QY, is 33,000 M⁻¹cm⁻¹. Superfolder GFP, a series of mutations that allow GFP to rapidly fold and mature even when fused to poorly folding peptides is also contemplated herein.

Many other mutations are contemplated, including color mutants; in particular, blue fluorescent protein (EBFP, EBFP2, Azurite, mKalamal), cyan fluorescent protein (ECFP, Cerulean, CyPet, mTurquoise2), and yellow fluorescent protein derivatives (YFP, Citrine, Venus, YPet). BFP derivatives (except mKalamal) contain the Y66H substitution. They exhibit a broad absorption band in the ultraviolet centered close to 380 nanometers and an emission maximum at 448 nanometers. A green fluorescent protein mutant (BFPms1) that preferentially binds Zn(II) and Cu(II) has been developed. BFPms1 have several important mutations including and the BFP chromophore (Y66H),Y145F for higher quantum yield, H148G for creating a hole into the beta-barrel and several other mutations that increase solubility. Zn(II) binding increases fluorescence intensity, while Cu(II) binding quenches fluorescence and shifts the absorbance maximum from 379 to 444 nm.

Because of the great variety of engineered GFP derivatives, fluorescent proteins that belong to a different family, such as the bilirubin-inducible fluorescent protein UnaG, dsRed, eqFP611, Dronpa, TagRFPs, KFP, EosFP, Dendra, IrisFP and many others, are erroneously referred to as GFP derivatives however each is contemplated herein, provided that they are not toxic to the plant cell (which can be easily determined).

Other fluorescent proteins (reporters) contemplated herein are provided below.

FMN-binding fluorescent proteins (FbFPs), a class of small (11-16 kDa), oxygen-independent fluorescent proteins that are derived from blue-light receptors.

A new class of fluorescent protein was evolved from a cyanobacterial (Trichodesmium erythraeum) phycobiliprotein, α-allophycocyanin, and named small ultra red fluorescent protein (smURFP) in 2016. smURFP autocatalytically self-incorporates the chromophore biliverdin without the need of an external protein, known as a lyase. Jellyfish- and coral-derived fluorescent proteins require oxygen and produce a stoichiometric amount of hydrogen peroxide upon chromophore formation. smURFP does not require oxygen or produce hydrogen peroxide and uses the chromophore, biliverdin. smURFP has a large extinction coefficient (180,000 M⁻¹ cm⁻¹) and has a modest quantum yield (0.20), which makes it comparable biophysical brightness to eGFP and ˜2-fold brighter than most red or far-red fluorescent proteins derived from coral. smURFP spectral properties are similar to the organic dye Cy5.

A review of new classes of fluorescent proteins and applications can be found in Trends in Biochemical Sciences [Rodriguez, Erik A.; Campbell, Robert E.; Lin, John Y; Lin, Michael Z.; Miyawaki, Atsushi; Palmer, Amy E.; Shu, Xiaokun; Zhang, Jin; Tsien, Roger E “The Growing and Glowing Toolbox of Fluorescent and Photoactive Proteins”. Trends in Biochemical Sciences. doi:10.1016/j.tibs.2016.09.010].

In certain embodiments, the nucleic acid construct is a non-integrating construct, preferably where the nucleic acid sequence encoding the fluorescent reporter is also non-integrating. As used herein, “non-integrating” refers to a construct or sequence that is not affirmatively designed to facilitate integration of the construct or sequence into the genome of the plant of interest. For example, a functional T-DNA vector system for Agrobacterium-mediated genetic transformation is not a non-integrating vector system as the system is affirmatively designed to integrate into the plant genome. Similarly, a fluorescent reporter gene sequence or selectable marker sequence that has flanking sequences that are homologous to the genome of the plant of interest to facilitate homologous recombination of the fluorescent reporter gene sequence or selectable marker sequence into the genome of the plant of interest would not be a non-integrating fluorescent reporter gene sequence or selectable marker sequence.

Typically, the nucleic acid construct is a nucleic acid expression construct.

The nucleic acid construct (also referred to herein as an “expression vector”, “vector” or “construct”) of some embodiments of the invention includes additional sequences which render this vector suitable for replication in prokaryotes, eukaryotes, or preferably both (e.g., shuttle vectors). To express a functional editing agent, the nuclease may not be sufficient, in cases where the cleaving module (nuclease) is not an integral part of the recognition unit. In such a case, the nucleic acid construct may also encode the recognition unit, which in the case of CRISPR-Cas is the gRNA. Alternatively, the gRNA can be cloned into a separate vector onto which a fluorescent reporter (preferably different than that cloned with the nuclease) is cloned as described herein. In such a case, at least two different vectors with at least two different reporters must be transformed into the same plant cell. Alternatively, the gRNA (or any other DNA recognition module used, dependent on the editing system that is used) can be provided as RNA to the cell.

Examples of suggested configurations include, but are not limited to:

1) The fluorescent protein is fused to the nuclease (e.g., Cas9);
2) The fluorescent protein is fused to the nuclease (e.g., Cas9) and then, post-translational proteolytic cleavage separates them. In such a case, and according to some embodiments the fluorescent protein is fused to the endonuclease (e.g., Cas9) and a 2A cleaving peptide which is exogenously expressed, post translationally cleaves the nuclease from the fluorescent reporter, separating them into two separate individual and functional proteins, i.e., endonuclease; and fluorescent protein;
3) The fluorescent protein is fused to the nuclease (e.g., Cas9) and a T2A cleaving peptide which is expressed on the vector (or a separate vector) cleaves the nuclease from the fluorescent reporter;
4) The endonuclease (e.g., Cas9) and the fluorescent protein are expressed by the same promoter, but are translated separately using an internal ribosome entry site (IRES);
5) The endonuclease (e.g., Cas9) and the sgRNA are expressed by the same promoter and the recognition unit (e.g., sgRNA) is cleaved out by ribozyme.

Typical cloning vectors may also contain a transcription and translation initiation sequence, transcription and translation terminator and optionally a polyadenylation signal.

According to a specific embodiment, the vector needs not comprise a selection marker (e.g., antibiotics selection marker).

According to a specific embodiment, each of the nucleic acid sequences encoding the genome editing agent and the nucleic acid sequence encoding the fluorescent reporter is operatively linked to a terminator (e.g., CaMV-35S terminator).

Constructs useful in the methods according to some embodiments of the invention may be constructed using recombinant DNA technology well known to persons skilled in the art. The nucleic acid sequences may be inserted into vectors, which may be commercially available, suitable for transforming into plants and suitable for transient expression of the gene of interest in the transformed cells. The genetic construct can be an expression vector wherein said nucleic acid sequence is operably linked to one or more regulatory sequences allowing expression in the plant cells.

In a particular embodiment of some embodiments of the invention the regulatory sequence is a plant-expressible promoter.

As used herein the phrase “plant-expressible” refers to a promoter sequence, including any additional regulatory elements added thereto or contained therein, that is at least capable of inducing, conferring, activating or enhancing expression in a plant cell, tissue or organ, preferably a monocotyledonous or dicotyledonous plant cell, tissue, or organ. Examples of preferred promoters useful for the methods of some embodiments of the invention are presented in Table I, below.

TABLE 1
Exemplary constitutive promoters for use in the
performance of some embodiments of the invention

| Gene | Expression | |
|---|---|---|
| Source | Pattern | Reference |
| Actin | constitutive | McElroy et al, Plant Cell, 2: |
| | | 163-171, 1990 |
| CaMV 35S | constitutive | Odell et al, Nature, 313: |
| | | 810-812, 1985 |
| CaMV 19S | constitutive | Nilsson et al., Physiol. Plant 100: |
| | | 456-462, 1997 |
| GOS2 | constitutive | de Pater et al, Plant J Nov; 2(6): |
| | | 837-44, 1992 |
| ubiquitin | constitutive | Christensen et al, Plant Mol. Biol. 18: |
| | | 675-689, 1992 |
| Rice | constitutive | Bucholz et al, Plant Mol Biol. 25(5): |
| cyclophilin | | 837-43, 1994 |
| Maize H3 | constitutive | Lepetit et al, Mol. Gen. Genet. 231: |
| histone | | 276-285, 1992 |
| Actin 2 | constitutive | An et al, Plant J. 10(1); |
| | | 107121, 1996 |
| CVMV | constitutive | Lawrenson et al, Gen Biol 16: |
| (Cassava Vein | | 258, 2015 |
| Mosaic Virus | | |
| U6 (AtU626; | constitutive | Lawrenson et al, Gen Biol 16: |
| TaU6) | | 258, 2015 |

According to a specific embodiment, promoters in the nucleic acid construct are identical (e.g., all identical, at least two identical).

According to a specific embodiment, promoters in the nucleic acid construct are different (e.g., at least two are different, all are different).

According to a specific embodiment, promoters in the nucleic acid construct comprise a Pol3 promoter. Examples of Pol3 promoters include, but are not limited to, AtU6-29, AtU626, AtU3B, AtU3d, TaU6.

According to a specific embodiment, promoters in the nucleic acid construct comprise a Pol2 promoter. Examples of Pol2 promoters include, but are not limited to, CaMV 35S, CaMV 19S, ubiquitin, CVMV.

According to a specific embodiment, promoters in the nucleic acid construct comprise a 35S promoter.

According to a specific embodiment, promoters in the nucleic acid construct comprise a U6 promoter.

According to a specific embodiment, promoters in the nucleic acid construct comprise a Pol 3 (e.g., U6) promoter operatively linked to the nucleic acid agent encoding at least one gRNA and/or a Pol2 (e.g., CamV35S) promoter operatively linked to said nucleic acid sequence encoding said genome editing agent or said nucleic acid sequence encoding said fluorescent reporter.

According to a specific embodiment, the construct is useful for transient expression (Helens et al., 2005, Plant Methods 1:13).

According to a specific embodiment, the nucleic acid sequences comprised in the construct are devoid or sequences which are homologous to the plant cell genome so as to avoid integration to the plant genome.

Methods of transient transformation are further described herein.

Various cloning kits can be used according to the teachings of some embodiments of the invention [e.g., GoldenGate assembly kit by New England Biolabs (NEB)].

According to a specific embodiment the nucleic acid construct is a binary vector. Examples for binary vectors are pBIN19, pBI101, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery (e.g. transfection, electroporation, bombardment, viral inoculation) are: pGE-sgRNA (Zhang et al. Nat. Comms. 2016 7:12697), pJIT163-Ubi-Cas9 (Wang et al. Nat. Biotechnol 2004 32, 947-951), pICH47742::2x355-5′UTR-hCas9(STOP)-NOST (Belhan et al. Plant Methods 2013 11; 9(1):39), pAHC25 (Christensen, A.H. & P. H. Quail, 1996. Ubiquitin promoter-based vectors for high-level expression of selectable and/or screenable marker genes in monocotyledonous plants. Transgenic Research 5: 213-218), pHBT-sGFP(S65T)-NOS (Sheen et al. Protein phosphatase activity is required for light-inducible gene expression in maize, EMBO J. 12 (9), 3497-3505 (1993).

According to an aspect of the invention there is provided a method of selecting cells comprising a genome editing event, the method comprising:

(a) transforming cells of a plant of interest with the nucleic acid construct as described herein;

(b) selecting transformed cells exhibiting fluorescence emitted by the fluorescent reporter using flow cytometry or imaging;

(c) culturing the transformed cells comprising the genome editing event by the DNA editing agent for a time sufficient to lose expression of the DNA editing agent so as to obtain cells which comprise a genome editing event generated by the DNA editing agent but lack DNA encoding the DNA editing agent; and

According to some embodiments, the method further comprises validating in the transformed cells, loss of expression of the fluorescent reporter following step (c).

According to some embodiments, the method further comprises validating in the transformed cells loss, of expression of the DNA editing agent following step (c).

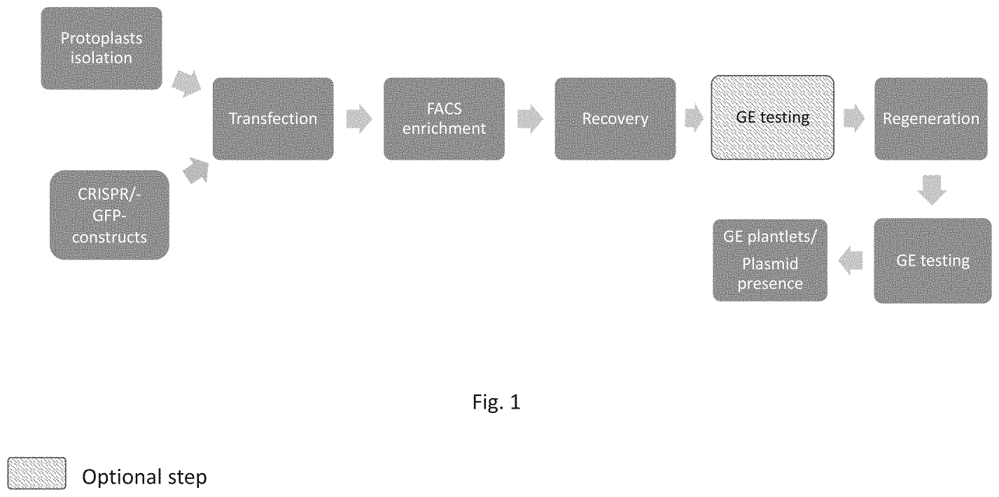

A non-limiting embodiment of the method is described in the Flowchart of FIG. 1.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores.

According to a specific embodiment, the plant or plant cell is non-transgenic [i.e., does not comprise heterologous sequence(s) integrated in the genome].

As used herein “heterologous” refers to non-naturally occurring either by way of composition (i.e., exogenous) or by way of position in the genome.

According to a specific embodiment, the plant part is a bean.

“Grain,” “seed,” or “bean,” refers to a flowering plant's unit of reproduction, capable of developing into another such plant. As used herein, especially with respect to coffee plants, the terms are used synonymously and interchangeably.

According to a specific embodiment, the cell is a germ cell.

According to a specific embodiment, the cell is a somatic cell.

The plant may be in any form including suspension cultures, protoplasts, embryos, meristematic regions, callus tissue, leaves, gametophytes, sporophytes, pollen, and microspores.

According to a specific embodiment, the plant part comprises DNA.

Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (<Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to a specific embodiment, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam.

According to a specific embodiment, the plant is asexually propagated.

According to a specific embodiment, the plant is banana.

According to a specific embodiment, the plant has a juvenile period of at least 2 years (e.g., at least 3 years).

According to a specific embodiment, the plant is coffee.

As used herein a “coffee” refers to a plant of the family Rubiaceae, genus Coffea. There are many coffee species. Embodiments of the invention may refer to two primary commercial coffee species: Coffea Arabica (C. arabica), which is known as arabica coffee, and Coffea canephora, which is known as robusta coffee (C. robusta). Coffea liberica Bull. ex Hiern is also contemplated here which makes up 3% of the world coffee bean market. Also known as Coffea arnoldiana De Wild or more commonly as Liberian coffee. Coffees from the species Arabica are also generally called “Brazils” or they are classified as “other milds”. Brazilian coffees come from Brazil and “other milds” are grown in other high-grade coffee producing countries, which are generally recognized as including Colombia, Guatemala, Sumatra, Indonesia, Costa Rica, Mexico, United States (Hawaii), El Salvador, Peru, Kenya, Ethiopia and Jamaica. Coffea canephora, i.e. robusta, is typically used as a low-cost extender for arabica coffees. These robusta coffees are typically grown in the lower regions of West and Central Africa, India, Southeast Asia, Indonesia, and also Brazil. A person skilled in the art will appreciate that a geographical area refers to a coffee growing region where the coffee growing process utilizes identical coffee seedlings and where the growing environment is similar.

According to a specific embodiment, the coffee plant is of a coffee breeding line, more preferably an elite line.

According to a specific embodiment, the coffee plant is of an elite line.

According to a specific embodiment, the coffee plant is of a purebred line.

According to a specific embodiment, the coffee plant is of a coffee variety or breeding germplasm.

The term “breeding line”, as used herein, refers to a line of a cultivated coffee having commercially valuable or agronomically desirable characteristics, as opposed to wild varieties or landraces. The term includes reference to an elite breeding line or elite line, which represents an essentially homozygous, usually inbred, line of plants used to produce commercial Fi hybrids. An elite breeding line is obtained by breeding and selection for superior agronomic performance comprising a multitude of agronomically desirable traits. An elite plant is any plant from an elite line. Superior agronomic performance refers to a desired combination of agronomically desirable traits as defined herein, wherein it is desirable that the majority, preferably all of the agronomically desirable traits are improved in the elite breeding line as compared to a non-elite breeding line. Elite breeding lines are essentially homozygous and are preferably inbred lines.

The term “elite line”, as used herein, refers to any line that has resulted from breeding and selection for superior agronomic performance. An elite line preferably is a line that has multiple, preferably at least 3, 4 5, 6 or more (genes for) desirable agronomic traits as defined herein.

The terms “cultivar” and “variety” are used interchangeable herein and denote a plant with has deliberately been developed by breeding, e.g., crossing and selection, for the purpose of being commercialized, e.g., used by farmers and growers, to produce agricultural products for own consumption or for commercialization. The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and-or backcrossing.

A non-comprehensive list, of coffee varieties is provided herein:

Wild Coffee: This is the common name of “Coffea racemosa Lour” which is a coffee species native to Ethiopia.

Baron Goto Red: A coffee bean cultivar that is very similar to ‘Catuai Red’. It is grown at several sites in Hawaii.

Blue Mountain: Coffea arabica L. ‘Blue Mountain’. Also known commonly as Jamaican coffea or Kenyan coffea. It is a famous Arabica cultivar that originated in Jamaica but is now grown in Hawaii, PNG and Kenya. It is a superb coffee with a high quality cup flavor. It is characterized by a nutty aroma, bright acidity and a unique beef-bullion like flavor.

Bourbon: Coffea arabica L. ‘Bourbon’. A botanical variety or cultivar of Coffea Arabica which was first cultivated on the French controlled island of Bourbon, now called Reunion, located east of Madagascar in the Indian ocean.

Brazilian Coffea: Coffea arabica L. ‘Mundo Novo’. The common name used to identify the coffee plant cross created from the “Bourbon” and “Typica” varieties.

Caracol/Caracoli: Taken from the Spanish word Caracolillo meaning ‘seashell’ and describes the peaberry coffee bean.

Catimor: Is a coffee bean cultivar cross-developed between the strains of Caturra and Hibrido de Timor in Portugal in 1959. It is resistant to coffee leaf rust (Hemileia vastatrix). Newer cultivar selection with excellent yield but average quality.

Catuai: Is a cross between the Mundo Novo and the Caturra Arabica cultivars. Known for its high yield and is characterized by either yellow (Coffea arabica L. ‘Catuai Amarelo’) or red cherries (Coffea arabica L. ‘Catuai Vermelho’).

Caturra: A relatively recently developed sub-variety of the Coffea Arabica species that generally matures more quickly, gives greater yields, and is more disease resistant than the traditional “old Arabica” varieties like Bourbon and Typica.

Columbiana: A cultivar originating in Columbia. It is vigorous, heavy producer but average cup quality.

Congencis: Coffea Congencis—Coffee bean cultivar from the banks of Congo, it produces a good quality coffee but it is of low yield. Not suitable for commercial cultivation

Dewevreilt: Coffea Dewevreilt. A coffee bean cultivar discovered growing naturally in the forests of the Belgian Congo. Not considered suitable for commercial cultivation.

Dybowskiilt: Coffea Dybowskiilt. This coffee bean cultivar comes from the group of Eucoffea of inter-tropical Africa. Not considered suitable for commercial cultivation

Excelsa: Coffea Excelsa—A coffee bean cultivar discovered in 1904. Possesses natural resistance to diseases and delivers a high yield. Once aged it can deliver an odorous and pleasant taste, similar to var. Arabica.

Guadalupe: A cultivar of Coffea Arabica that is currently being evaluated in Hawaii.

Guatemala(n): A cultivar of Coffea Arabica that is being evaluated in other parts of Hawaii.

Hibrido de Timor: This is a cultivar that is a natural hybrid of Arabica and Robusta. It resembles Arabica coffee in that it has 44 chromosomes.

Icatu: A cultivar which mixes the “Arabica & Robusta hybrids” to the Arabica cultivars of Mundo Novo and Caturra.

Interspecific Hybrids: Hybrids of the coffee plant species and include; ICATU (Brazil; cross of Bourbon/MN & Robusta), 52828 (India; cross of Arabica & Liberia), Arabusta (Ivory Coast; cross of Arabica & Robusta).

‘K7’, ‘SL6’, ‘SL26’, ‘H66”, ‘KP532’: Promising new cultivars that are more resistant to the different variants of coffee plant disease like Hemileia.

Kent: A cultivar of the Arabica coffee bean that was originally developed in Mysore India and grown in East Africa. It is a high yielding plant that is resistant to the “coffee rust” decease but is very susceptible to coffee berry disease. It is being replaced gradually by the more resistant cultivar's of ‘S.288’, ‘S.333’ and ‘S.795’.

Kouillou: Name of a Coffea canephora (Robusta) variety whose name comes from a river in Gabon in Madagascar.

Laurina: A drought resistant cultivar possessing a good quality cup but with only fair yields.

Maragogipe/Maragogype: Coffea arabica L. ‘Maragopipe’. Also known as “Elephant Bean”. A mutant variety of Coffea Arabica (Typica) which was first discovered (1884) in Maragogype County in the Bahia state of Brazil.

Mauritiana: Coffea Mauritiana. A coffee bean cultivar that creates a bitter cup. Not considered suitable for commercial cultivation

Mundo Novo: A natural hybrid originating in Brazil as a cross between the varieties of ‘Arabica’ and ‘Bourbon’. It is a very vigorous plant that grows well at 3,500 to 5,500 feet (1,070 m to 1,525 m), is resistant to disease and has a high production yield. Tends to mature later than other cultivars.

Neo-Arnoldiana: Coffea Neo-Arnoldiana is a coffee bean cultivar that is grown in some parts of the Congo because of its high yield. It is not considered suitable for commercial cultivation.

Nganda: Coffea canephora Pierre ex A. Froehner ‘Nganda’. Where the upright form of the coffee plant Coffea Canephora is called Robusta its spreading version is also known as Nganda or Kouillou.

Paca: Created by El Salvador's agricultural scientists, this cultivar of Arabica is shorter and higher yielding than Bourbon but many believe it to be of an inferior cup in spite of its popularity in Latin America.

Pacamara: An Arabica cultivar created by crossing the low yield large bean variety Maragogipe with the higher yielding Paca. Developed in El Salvador in the 1960's this bean is about 75% larger than the average coffee bean.

Pache Colis: An Arabica cultivar being a cross between the cultivars Caturra and Pache comum. Originally found growing on a Guatemala farm in Mataquescuintla.

Pache Comum: A cultivar mutation of Typica (Arabica) developed in Santa Rosa

Guatemala. It adapts well and is noted for its smooth and somewhat flat cup

Preanger: A coffee plant cultivar currently being evaluated in Hawaii.

Pretoria: A coffee plant cultivar currently being evaluated in Hawaii.

Purpurescens: A coffee plant cultivar that is characterized by its unusual purple leaves. Racemosa: Coffea Racemosa—A coffee bean cultivar that looses its leaves during the dry season and re-grows them at the start of the rainy season. It is generally rated as poor tasting and not suitable for commercial cultivation.

Ruiru 11: Is a new dwarf hybrid which was developed at the Coffee Research Station at Ruiru in Kenya and launched on to the market in 1985. Ruiru 11 is resistant to both coffee berry disease and to coffee leaf rust. It is also high yielding and suitable for planting at twice the normal density.

San Ramon: Coffea arabica L. ‘San Ramon’. It is a dwarf variety of Arabica var typica. A small stature tree that is wind tolerant, high yield and drought resistant.

Tico: A cultivar of Coffea Arabica grown in Central America.

Timor Hybrid: A variety of coffee tree that was found in Timor in 1940s and is a natural occurring cross between the Arabica and Robusta species.

Typica: The correct botanical name is Coffea arabica L. ‘Typica’. It is a coffee variety of Coffea Arabica that is native to Ethiopia. Var Typica is the oldest and most well known of all the coffee varieties and still constitutes the bulk of the world's coffee production. Some of the best Latin-American coffees are from the Typica stock. The limits of its low yield production are made up for in its excellent cup.

Villalobos: A cultivar of Coffea Arabica that originated from the cultivar ‘San Ramon’ and has been successfully planted in Costa Rica.

As used herein the term “banana” refers to a plant of the genus Musa, including Plantains.

According to a specific embodiment, the banana is triploid.

Other ploidies are also contemplated, including, diploid and tetraploid.

Following is a non-limiting list of cultivars that can be used according to the present teachings.

AA Group

Diploid Musa acuminata, both wild banana plants and cultivars
Chingan banana
Lacatan banana
Lady Finger banana (Sugar banana)
Pisang jari buaya (Crocodile fingers banana)
Señorita banana (Monkoy, Arnibal banana, Cuarenta dias, Cariñosa, Pisang Empat Puluh Hari, Pisang Lampung)^{[12]}
Sinwobogi banana

AAA Group

Triploid Musa acuminata, both wild banana plants and cultivars

### Cavendish Subgroup

### ‘Dwarf Cavendish’

### ‘Giant Cavendish’ (‘Williams’)

### ‘Grand Nain’ (‘Chiquita’)

### ‘Masak Hijau’

### ‘Robusta’

### ‘Red Dacca’

Dwarf Red banana
Gros Michel banana
East African Highland bananas (AAA-EA subgroup)

AAAA Group

Tetraploid Musa acuminata, both wild bananas and cultivars
Bodles Altafort banana
Golden Beauty banana

AAAB Group

Tetraploid cultivars of Musa×paradisiaca
Atan banana
Goldfinger banana

AAB Group

Triploid cultivars of Musa×paradisiaca. This group contains the Plantain subgroup, composed of “true” plantains or African Plantains—whose centre of diversity is Central and West Africa, where a large number of cultivars were domesticated following the introduction of ancestral Plantains from Asia, possibly 2000-3000 years ago.

The Iholena and Maoli-Popo'ulu subgroups are referred to as Pacific plantains.
Iholena subgroup—subgroup of cooking bananas domesticated in the Pacific region
Maoli-Popo′ulu subgroup—subgroup of cooking bananas domesticated in the Pacific region
Maqueño banana
Popoulu banana
Mysore subgroup—cooking and dessert bananas^{[15]}
Mysore banana
Pisang Raja subgroup
Pisang Raja banana
Plantain subgroup
French plantain
Green French banana
Horn plantain & Rhino Horn banana
Nendran banana
Pink French banana
Tiger banana
Pome subgroup
Pome banana
Prata-anã banana (Dwarf Brazilian banana, Dwarf Prata)
Silk subgroup
Latundan banana (Silk banana, Apple banana)

### Others

Pisang Seribu banana
plu banana

AABB Group

Tetraploid cultivars of Musa×paradisiaca
Kalamagol banana
Pisang Awak (Ducasse banana)

AB Group

Diploid cultivars of Musa×paradisiaca
Ney Poovan banana

ABB Group

Triploid cultivars of Musa×paradisiaca
Blue Java banana (Ice Cream banana, Ney mannan, Ash plantain, Pata hina, Dukuru, Vata)

### Bluggoe Subgroup

Bluggoe banana (also known as orinoco and “burro”)
Silver Bluggoe banana
Pelipita banana (Pelipia, Pilipia)

### Saba Subgroup

Saba banana (Cardaba, Dippig)
Cardaba banana
Benedetta banana

ABBB Group

Tetraploid cultivars of Musa×paradisiaca
Tiparot banana

BB Group

Diploid Musa balbisiana, wild bananas

BBB Group

Triploid Musa balbisiana, wild bananas and cultivars

### Kluai Lep Chang Kut

According to a specific embodiment, the plant is a plant cell e.g., plant cell in an embryonic cell suspension.

According to a specific embodiment, the plant cell is a protoplast.

The protoplasts are derived from any plant tissue e.g., roots, leaves, embryonic cell suspension, calli or seedling tissue.

According to a specific embodiment, the genome editing event comprises a deletion, a single base pair substitution, or an insertion of genetic material from a second plant that could otherwise be introduced into the plant of interest by traditional breeding.

According to a specific embodiment, the genome editing event does not comprise an introduction of foreign DNA into a genome of the plant of interest that could not be introduced through traditional breeding.

There are a number of methods of introducing DNA into plant cells e.g., using protoplasts and the skilled artisan will know which to select.

The delivery of nucleic acids may be introduced into a plant cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into plant cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mae et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, the introduction of DNA into plant cells (e.g., protoplasts) is effected by electroporation.

According to a specific embodiment, the introduction of DNA into plant cells (e.g., protoplasts) is effected by bombardment/biolistics.

According to a specific embodiment, for introducing DNA into protoplasts the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373. Protoplasts are then cultured under conditions that allowed them to grow cell walls, start dividing to form a callus, develop shoots and roots, and regenerate whole plants.

Transient transformation can also be effected by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

Regardless of the transformation/infection method employed, the present teachings further relate to any cell e.g., a plant cell (e.g., protoplast) or a bacterial cell comprising the nucleic acid construct(s) as described herein.

Following transformation, cells are subjected to flow cytometry to select transformed cells exhibiting fluorescence emitted by the fluorescent reporter.

This analysis is typically effected within 24-72 hours e.g., 48-72, 24-28 hours, following transformation. To ensure transient expression, no marker selection is employed e.g., antibiotics for a selection marker. The culture may still comprise antibiotics but not to a selection marker.

Flow cytometry of plant cells is typically performed by Fluorescence Activated Cell Sorting (FACS). Fluorescence activated cell sorting (FACS) is a well-known method for separating particles, including cells, based on the fluorescent properties of the particles (see, e.g., Kamarch, 1987, Methods Enzymol, 151:150-165).

For instance, FACS of GFP-positive cells makes use of the visualization of the green versus the red emission spectra of protoplasts excited by a 488 nm laser. GFP-positive protoplasts can be distinguished by their increased ratio of green to red emission.

Following is a non-binding protocol adapted from Bastiaan et al. J Vis Exp. 2010; (36): 1673, which is hereby incorporated by reference. FACS apparati are commercially available e.g., FACSMelody (BD), FACSAria (BD).

A flow stream is set up with a 100 μm nozzle and a 20 psi sheath pressure. The cell density and sample injection speed can be adjusted to the particular experiment based on whether a best possible yield or fastest achievable speed is desired, e.g., up to 10,000,000 cells/ml. The sample is agitated on the FACS to prevent sedimentation of the protoplasts. If clogging of the FACS is an issue, there are three possible troubleshooting steps: 1. Perform a sample-line backflush. 2. Dilute protoplast suspension to reduce the density. 3. Clean up the protoplast solution by repeating the filtration step after centrifugation and resuspension. The apparatus is prepared to measure forward scatter (FSC), side scatter (SSC) and emission at 530/30 nm for GFP and 610/20 nm for red spectrum auto-fluorescence (RSA) after excitation by a 488 nm laser. These are in essence the only parameters used to isolate GFP-positive protoplasts. The voltage settings can be used: FSC-60V, SSC 250V, GFP 350V and RSA 335V. Note that the optimal voltage settings will be different for every FACS and will even need to be adjusted throughout the lifetime of the cell sorter.

The process is started by setting up a dotplot for forward scatter versus side scatter. The voltage settings are applied so that the measured events are centered in the plot. Next, a dot plot is created of green versus red fluorescence signals. The voltage settings are applied so that the measured events yield a centered diagonal population in the plot when looking at a wild-type (non-GFP) protoplast suspension. A protoplast suspension derived from a GFP marker line will produce a clear population of green fluorescent events never seen in wild-type samples. Compensation constraints are set to adjust for spectral overlap between GFP and RSA. Proper compensation constraint settings will allow for better separation of the GFP-positive protoplasts from the non-GFP protoplasts and debris. The constraints used here are as follows: RSA, minus 17.91% GFP. A gate is set to identify GFP-positive events, a negative control of non-GFP protoplasts should be used to aid in defining the gate boundaries. A forward scatter cutoff is implemented in order to leave small debris out of the analysis. The GFP-positive events are visualized in the FSC vs. SSC plot to help determine the placement of the cutoff. E.g., cutoff is set at 5,000. Note that the FACS will count debris as sort events and a sample with high levels of debris may have a different percent GFP positive events than expected. This is not necessarily a problem. However, the more debris in the sample, the longer the sort will take. Depending on the experiment and the abundance of the cell type to be analyzed, the FACS precision mode is set either for optimal yield or optimal purity of the sorted cells.

Following FACS sorting, positively selected pools of transformed plant cells, (e.g., protoplasts) displaying the fluorescent marker are collected and an aliquot can be used for testing the DNA editing event (optional step, see FIG. 1). Alternatively (or following optional validating) the clones are cultivated in the absence of selection (e.g., antibiotics for a selection marker) until they develop into colonies i.e., clones (at least 28 days) and micro-calli. Following at least 60-100 days in culture (e.g., at least 70 days, at least 80 days), a portion of the cells of the calli are analyzed (validated) for: the DNA editing event and the presence of the DNA editing agent, namely, loss of DNA sequences encoding for the DNA editing agent, pointing to the transient nature of the method.

Thus, clones are validated for the presence of a DNA editing event also referred to herein as “mutation” or “edit”, dependent on the type of editing sought e.g., insertion, deletion, insertion-deletion (Indel), inversion, substitution and combinations thereof.

Methods for detecting sequence alteration are well known in the art and include, but not limited to, DNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Hetroduplex and Sanger sequencing.

Another method of validating the presence of a DNA editing event e.g., Indels comprises a mismatch cleavage assay that makes use of a structure selective enzyme (e,g,m endonuclease) that recognizes and cleaves mismatched DNA.

The mismatch cleavage assay is a simple and cost-effective method for the detection of indels and is therefore the typical procedure to detect mutations induced by genome editing. The assay uses enzymes that cleave heteroduplex DNA at mismatches and extrahelical loops formed by multiple nucleotides, yielding two or more smaller fragments. A PCR product of −300-1000 bp is generated with the predicted nuclease cleavage site off-center so that the resulting fragments are dissimilar in size and can easily be resolved by conventional gel electrophoresis or high-performance liquid chromatography (HPLC). End-labeled digestion products can also be analyzed by automated gel or capillary electrophoresis. The frequency of indels at the locus can be estimated by measuring the integrated intensities of the PCR amplicon and cleaved DNA bands. The digestion step takes 15-60 min, and when the DNA preparation and PCR steps are added the entire assays can be completed in <3 h.

Two alternative enzymes are typically used in this assay. T7 endonuclease 1 (T7E1) is a resolvase that recognizes and cleaves imperfectly matched DNA at the first, second or third phosphodiester bond upstream of the mismatch. The sensitivity of a T7E1-based assay is 0.5-5%. In contrast, Surveyor™ nuclease (Transgenomic Inc., Omaha, Nebr., USA) is a member of the CEL family of mismatch-specific nucleases derived from celery. It recognizes and cleaves mismatches due to the presence of single nucleotide polymorphisms (SNPs) or small indels, cleaving both DNA strands downstream of the mismatch. It can detect indels of up to 12 nt and is sensitive to mutations present at frequencies as low as ˜3%, i.e. 1 in 32 copies.

Yet another method of validating the presence of an editing even comprises the high-resolution melting analysis.

High-resolution melting analysis (HRMA) involves the amplification of a DNA sequence spanning the genomic target (90-200 bp) by real-time PCR with the incorporation of a fluorescent dye, followed by melt curve analysis of the amplicons. HRMA is based on the loss of fluorescence when intercalating dyes are released from double-stranded DNA during thermal denaturation. It records the temperature-dependent denaturation profile of amplicons and detects whether the melting process involves one or more molecular species.

Yet another method is the heteroduplex mobility assay. Mutations can also be detected by analyzing re-hybridized PCR fragments directly by native polyacrylamide gel electrophoresis (PAGE). This method takes advantage of the differential migration of heteroduplex and homoduplex DNA in polyacrylamide gels. The angle between matched and mismatched DNA strands caused by an indel means that heteroduplex DNA migrates at a significantly slower rate than homoduplex DNA under native conditions, and they can easily be distinguished based on their mobility. Fragments of 140-170 bp can be separated in a 15% polyacrylamide gel. The sensitivity of such assays can approach 0.5% under optimal conditions, which is similar to T7E1 (After reannealing the PCR products, the electrophoresis component of the assay takes ˜2 h.

Other methods of validating the presence of editing events are described in length in Zischewski 2017 Biotechnol. Advances 1(1):95-104.

It will be appreciated that positive clones can be homozygous or heterozygous for the DNA editing event. The skilled artisan will select the clone for further culturing/regeneration according to the intended use.

Clones exhibiting the presence of a DNA editing event as desired are further analyzed for the presence of the DNA editing agent. Namely, loss of DNA sequences encoding for the DNA editing agent, pointing to the transient nature of the method.

This can be done by analyzing the expression of the DNA editing agent (e.g., at the mRNA, protein) e.g., by fluorescent detection of GFP or q-PCR, HPLC.

Alternatively or additionally, the cells are analyzed for the presence of the nucleic acid construct as described herein or portions thereof e.g., nucleic acid sequence encoding the reporter polypeptide or the DNA editing agent.

Clones showing no DNA encoding the fluorescent reporter or DNA editing agent (e.g., as affirmed by fluorescent microscopy, q-PCR and or any other method such as Southern blot, PCR, sequencing, HPLC) yet comprising the DNA editing event(s) [mutation(s)] as desired are isolated for further processing.

These clones can therefore be stored (e.g., cryopreserved).

Alternatively, cells (e.g., protoplasts) may be regenerated into whole plants first by growing into a group of plant cells that develops into a callus and then by regeneration of shoots (caulogenesis) from the callus using plant tissue culture methods. Growth of protoplasts into callus and regeneration of shoots requires the proper balance of plant growth regulators in the tissue culture medium that must be customized for each species of plant

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the gene editing event(s) generated according to the present teachings.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals in between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### General Materials and Methods

### Embryogenic Callus and Cell Suspension Generation and Maintenance

Embryonic calli were obtained as previously described [Etienne, H., Somatic embryogenesis protocol: coffee (Coffea arabica L. and C. canephora P.), in Protocol for somatic embryogenesis in woody plants. 2005, Springer. p. 167-1795]. Briefly, young leaves were surface sterilized, cut into 1 cm² pieces and placed on half strength semi solid MS medium supplemented with 2.26 μM 2,4-dichlorophenoxyacetic acid (2,4-D), 4.92 μM indole-3-butyric acid (IBA) and 9.84 μM isopentenyladenine (iP) for one month. Explants were then transferred to half strength semisolid MS medium containing 4.52 μM 2,4-D and 17.76 μM 6-benzylaminopurine (6-BAP) for 6 to 8 months until regeneration of embryogenic calli. Embryogenic calli were maintained on MS media supplemented with 5 μM 6-BAP.

Cell suspension cultures were generated from embryogenic calli as previously described [Acuna, J. R. and M. de Pena, Plant regeneration from protoplasts of embryogenic cell suspensions of Coffea arabica L. cv. caturra. Plant Cell Reports, 1991. 10(6): p. 345-348]. Embryogenic calli (30 g/l) were placed in liquid MS medium supplemented with 13.32 μM 6-BAP. Flasks were placed in a shaking incubator (110 rpm) at 28° C. The cell suspension was subcultured/passaged every two to four weeks until fully established. Cell suspension cultures were maintained in liquid MS medium with 4.44 μM 6-BAP.

Target Genes Phytoene desaturase gene (PDS).

Rationale:

PDS is an essential gene in the chlorophyll biosynthesis pathway and loss of PDS function in plants results in albino phenotype (Fan D et al. 2015 Sci Rep 20, 5:12217). When used as a target gene in genome editing (GE) strategy, positively edited plants are easily identified by partial or complete loss of chlorophyll in leaves and other organs.

Methods:

sgRNAs targeting the PDS gene from banana and coffee are designed and cloned (see Table 2). Following transfection and FACS sorting, protocolonies (or calli) that tested positive for DNA editing and negative for the presence of Cas9 are transferred into solid regeneration media (half strength MS+B5 vitamins, 20 g/l sucrose, 0.8% agar) until shoots are regenerated. Loss of pigmentation in these shoots indicates loss of function of the PDS gene and correct GE. No albino phenotype is observed in the control plantlets transfected with an empty vector.

CLA1 gene.

Rationale:

CLA1 encodes the first enzyme of the 2-C-methyl-Derythriol-4-phosphate pathway and loss of function in this gene interferes with the normal development of chrloroplasts, resulting in albino plant tissues (Gao et al 2011 Plant J 66, 2:293). When used as a target gene in GE strategy, positively edited plants are easily identified by partial or complete loss of chlorophyll in leaves and other organs.

Methods:

sgRNAs targeting the CLA1 gene from banana and coffee were designed and cloned (see Table 2). Following transfection and FACS sorting, protocolonies (or calli) that tested positive for DNA editing and negative for the presence of Cas9 are transferred into solid regeneration media (half strength MS+B5 vitamins, 20 g/l sucrose, 0.8% agar) until shoots are regenerated. Loss of pigmentation in these shoots indicates loss of function of the CLA1 gene and correct GE. No albino phenotype is observed in the control plantlets transfected with an empty vector.

TOR1 (tortifolia 1) gene.

Rationale:

TOR1 is a plant-specific microtubule associated protein that regulates the orientation of cortical microtubules and the direction of organ growth. Loss of TOR1 function leads to a striking twisting of leaf petioles resulting in right-handed displacement of the leaf blades and helical growth (Buschmann et al 2004 Curr Biol 14, 16:1515).

sgRNAs Design

sgRNAs are designed using the publically available sgRNA designer, from Park, J., S. Bae, and J.-S. Kim, Cas-Designer: a web-based tool for choice of CRISPR-Cas9 target sites. Bioinformatics, 2015. 31(24): p. 4014-4016. Two sgRNAs are designed for each gene to increase the chances of a DSBs which could result in the loss of function of the target gene.

TABLE 2
Target Genes IDs

| | | | Banana gene 1 | Banana gene 2 | | |
|---|---|---|---|---|---|---|
| | | Query | ID and identity | ID and identity | Coffee gene ID and | |
| Gene | Query sequence | sequence | (%) to Query/ | (%) to Query/ | identity (%) to | sgRNA (SEQ |
| name | ID | organism | SEQ ID NO: | SEQ ID NO: | Query/SEQ ID NO: | ID NO:) |
| PDS | Solyc03g123760.2 | Solanum | Ma08_p16510.2 | Ma08_p16510.1 | Cc04_g00540 (82%) | 10-13, 25, |
| | | lycopersicum | (75%) | (77%) | | 28, 29 |
| | | (tomato) | | | | |
| CLA1 | AT4G15560 | Arabidopsis | Ma10_p01930.1 | Ma03_p26140.1 | Cc03_g02540 (88%) | 14-21, 26, |
| | | thaliana | (81%) | (82%) | | 30, 31 |
| | Solyc01g067890.2.1 | Solanum | Ma10_p01930.1 | Ma03_p26140.1 | Cc03_g02540 (84%) | |
| | | lycopersicum | (83%) | (85%) | | |
| TOR1 | AT4G27060 | Arabidopsis | Ma09_p11270.1 | Ma09_p02740.1 | Cc05_g13520 (56%) | 822-24, 27, |
| | | thaliana | (50%) | (49%) | | 32, 33 |
| | Solyc10g006350.2.1 | Solanum | Ma09_p11270.1 | Ma09_p02740.1 | Cc05_g13520 (71%) | |
| | | lycopersicum | (57%) | (54%) | | |
| | | | AT4G27060/ | | | |
| | | | Solyc10g006350.2.1 | | | |
| | | | identity: | | | |
| | | | 57% | | | |
| eGFP | AFA52654 | Aequorea | | | | 34, 35 |
| | | victoria | | | | |

sgRNA Cloning

The transfection plasmid utilized was composed of 4 modules comprising of 1, eGFP driven by the CaMV35s promoter terminated by a G7 temination sequence; 2, Cas9 (human codon optimised) driven by the CaMV35s promoter terminated by Mas termination sequence; 3, AtU6 promoter driving sgRNA for guide 1; 4 AtU6 promoter driving sgRNA for guide 2. A binary vector can be used such as pCAMBIA or pRI-201-AN DNA.

Cas9 and/or sgRNA Plasmid Optimization by Targeting Exogenous Reporter Gene GFP

To analyze the strength of different RNA polymerase III (pol-III) promoters sgRNA were designed for targeting eGFP in the CRISPR Cas9 complex and then the effect of different promoters in knocking out eGFP expression in transformed cells was tested.

Specifically, plasmids (e.g. pBluescript, pUC19) contained four transcriptional units containing Cas9, eGFP, dsRED, and sgRNA-GFP driven by different pol-II and pol-III promoters (e.g. CAMV 35S, U6) These plasmids were transfected into protoplast cultures and analyzed by FACS after a 24-72 hour incubation period. High frequency in dsRED (or mCherry, RFP) expression indicated high transfection efficiency, while low frequency in eGFP expression indicated successful gene editing through CRISPR-Cas9. Therefore the line that showed the lowest eGFP:dsRED expression ratio was the chosen pol-III promoter as it caused the highest proportion of eGFP inactivation through CRISPR Cas9 complexes.

Final Plasmid Design

For transient expression, a plasmid containing four transcriptional units was used. The first transcriptional unit contained the CaMV-35S promoter-driving expression of Cas9 and the tobacco mosaic virus (TMV) terminator. The next transcriptional unit consisted of another CaMV-35S promoter driving expression of eGFP and the nos terminator. The third and fourth transcriptional units each contained the Arabidopsis U6 promoter expressing sgRNA to target genes (as mentioned each vector comprises two sgRNAs).

Protoplasts Isolation

Protoplasts were isolated by incubating plant material (e.g. leaves, calli, cell suspensions) in a digestion solution (1% cellulase, 0.5% macerozyme, 0.5% driselase, 0.4M mannitol, 154 mM NaCl, 20 mM KCl, 20 mM MES pH 5.6, 10 mM CaCl2) for 4-24 h at room temperature and gentle shaking. After digestion, remaining plant material was washed with W5 solution (154 mM NaCl, 125 mM CaCl2, 5 mM KCl, 2 mM MES pH5.6) and protoplasts suspension was filtered through a 40 um strainer. After centrifugation at 80 g for 3 min at room temperature, protoplasts were resuspended in 2 ml W5 buffer and precipitated by gravity in ice. The final protoplast pellet was resuspended in 2 ml of MMG (0.4M mannitol, 15 mM MagC12, 4 mM MES pH 5.6) and protoplast concentration was determined using a hemocytometer. Protoplasts viability was estimated using Trypan Blue staining.

Polyethylene glycol (PEG)-mediated plasmid transfection. PEG-transfection of coffee and banana protoplasts was effected using a modified version of the strategy reported by Wang et al. (2015) [Wang, H., et al., An efficient PEG-mediated transient gene expression system in grape protoplasts and its application in subcellular localization studies of flavonoids biosynthesis enzymes. Scientia Horticulturae, 2015. 191: p. 82-89]. Protoplasts were resuspended to a density of 2-5×10⁶ protoplasts/ml in MMg solution. 100-200 μl of protoplast suspension was added to a tube containing the plasmid. The plasmid:protoplast ratio greatly affects transformation efficiency therefore a range of plasmid concentrations in protoplast suspension, 5-300 μg/μ1, were assayed. PEG solution (100-200 μl) was added to the mixture and incubated at 23° C. for various lengths of time ranging from 10-60 minutes. PEG4000 concentration was optimized, a range of 20-80% PEG4000 in 200-400 mM mannitol, 100-500 mM CaCl₂) solution was assayed. The protoplasts were then washed in W5 and centrifuged at 80 g for 3 min, prior resuspension in 1 ml W5 and incubated in the dark at 23° C. After incubation for 24-72 h fluorescence was detected by microscopy.

Electroporation

A plasmid containing Pol2-driven GFP/RFP, Pol2-driven-NLS-Cas9 and Pol3-driven sgRNA targeting the relevant genes (see list of Table 2 above) was introduced to the cells using electroporation (BIORAD-GenePulserII; Miao and Jian 2007 Nature Protocols 2(10): 2348-2353. 500 μl of protoplasts were transferred into electroporation cuvettes and mixed with 100 μl of plasmid (10-40 μg DNA). Protoplasts were electroporated at 130 V and 1,000 F and incubated at room temperature for 30 minutes. 1 ml of protoplast culture medium was added to each cuvette and the protoplast suspension was poured into a small petri dish. After incubation for 24-48 h fluorescence was detected by microscopy.

FACS Sorting of Fluorescent Protein-Expressing Cells

48 hrs after plasmid/RNA delivery, cells were collected and sorted for fluorescent protein expression using a flow cytometer in order to enrich for GFP/Editing agent expressing cells [Chiang, T. W., et al., CRISPR-Cas9(D10A) nickase-based genotypic and phenotypic screening to enhance genome editing. Sci Rep, 2016. 6: p. 24356]. This enrichment step allows bypassing antibiotic selection and collecting only cells transiently expressing the fluorescent protein, Cas9 and the sgRNA. These cells can be further tested for editing of the target gene by non-homologues end joining (NHEJ) and loss of the corresponding gene expression.

Colony Formation

The fluorescent protein positive cells were partly sampled and used for DNA extraction and genome editing (GE) testing and partly plated at high dilution in liquid medium to allow colony formation for 28-35 days. Colonies were picked, grown and split into two aliquots. One aliquot was used for DNA extraction and genome editing (GE) testing and CRISPR DNA-free testing (see below), while the others were kept in culture until their status was verified. Only the ones clearly showing to be GE and CRISPR DNA-free were selected forward.

After 20 days in the dark (from splitting for GE analysis, i.e., 60 days, hence 80 days in total), the colonies were transferred to the same medium but with reduced glucose (0.46 M) and 0.4% agarose and incubated at a low light intensity. After six weeks agarose was cut into slices and placed on protoplast culture medium with 0.31 M glucose and 0.2% gelrite. After one month, protocolonies (or calli) were subcultured into regeneration media (half strength MS+B5 vitamins, 20 g/l sucrose). Regenerated plantlets were placed on solidified media (0.8% agar) at a low light intensity at 28° C. After 2 months plantlets were transferred to soil and placed in a glasshouse at 80-100% humidity.

Screen for Gene Modification and Absence of CRISPR System DNA

From each colony DNA was extracted from an aliquot of GFP-sorted protoplasts (optional step) and from protoplasts-derived colonies and a PCR reaction was performed with primers flanking the targeted gene. Measures are taken to sample the colony as positive colonies will be used to regenerate the plant. A control reaction from protoplasts subjected to the same method but without Cas9-sgRNA is included and considered as wild type (WT). The PCR products were then separated on an agarose gel to detect any changes in the product size compared to the WT. The PCR reaction products that vary from the WT products were cloned into pBLUNT or PCR-TOPO (Invitrogen). Alternatively, sequencing was used to verify the editing event. The resulting colonies were picked, plasmids were isolated and sequenced to determine the nature of the mutations. Clones (colonies or calli) harbouring mutations that were predicted to result in domain-alteration or complete loss of the corresponding protein were chosen for whole genome sequencing in order to validate that they were free from the CRISPR system DNA/RNA and to detect the mutations at the genomic DNA level.

Positive clones exhibiting the desired GE were first tested for GFP expression via microscopy analysis (compared to WT). Next, GFP-negative plants were tested for the presence of the Cas9 cassette by PCR using primers specific (or next generation sequencing, NGS) for the Cas9 sequence or any other sequence of the expression cassette. Other regions of the construct can also be tested to ensure that nothing of the original construct is in the genome.

Plant Regeneration

Clones that were sequenced and predicted to have lost the expression of the target genes and found to be free of the CRISPR system DNA/RNA were propagated for generation in large quantities and in parallel were differentiated to generate seedlings from which functional assay is performed to test the desired trait.

Phenotypic Analysis

As described above, such as by looking at the pigmentation or morphology dependent on the target gene.

### Example 2

### FACS Enrichment of Cells Expressing Fluorescent Reporter in

Banana and Coffee

TABLE 3
sgRNAs used in this Example are provided in Table 3 below.

| Species | Gene | Gene ID | sgRNA ID | sgRNA sequence |
|---|---|---|---|---|
| Musa | PDS | Ma08_g16510 | sgRNA224 | GACTAGAGATGTCCTGT/ |
| acuminata | | | | SEQ ID NO: 66 |
| | | | sgRNA227 | CATCTTTCTGCAATTCCAC/ |
| | | | | SEQ ID NO: 67 |
| | | | sgRNA228 | GTCTCTCCCATGAAGTTAAGT/ |
| | | | | SEQ ID NO: 68 |
| Coffea | PDS | Cc04_g00540 | sgRNA165 | TTTCTGCACTAAGCCTGACCA/ |
| canephora | | | | SEQ ID NO: 69 |
| | | | sgRNA166 | TTTATTGATTCTATG// |
| | | | | SEQ ID NO: 70 |
| | | | sgRNA167 | TGAAAATGCCGTCAACTATTT// |
| | | | | SEQ ID NO: 71 |
| | | | sgRNA168 | CCGTACTTCTCCTCATCCAAATA/ |
| | | | | SEQ ID NO: 72 |
| N/A | eGFP | N/A | sgRNA- | GCGAAGCTGTTCACCG/ |
| | | | eGFP1 | SEQ ID NO: 73 |
| N/A | eGFP | N/A | sgRNA- | CCACAAGTTCAGCGTGTC/ |
| | | | eGFP3 | SEQ ID NO: 74 |

A robust protocols for to efficient isolation of protoplasts from Coffea species' calli and/or cell suspensions and Musa acuminata cells suspensions was developed to subsequently transfect them with plasmids carrying the CRISPR/Cas9 machinery to target genes of interest (e.g. PDS as an endogenous gene or GFP as an exogenous gene, also termed as a reporter sensor plasmid) and enrich for cells expressing a reporter using FACS sorting. To achieve this aim, the present inventors (i) generated and maintained embryogenic material; (ii) isolated protoplasts from that material; (iii) transfected with specific plasmids targeting PDS or a reporter-sensor plasmid (e.g., eGFP); (iv) enriched for cells expressing a fluorescent marker as a proxy for cells (e.g., mCherry) that carry the CRISPR/Cas9 complex and sgRNAs that target the gene of interest or a reporter-sensor plasmid; and (v) advanced sorted protoplasts through our protoplast-regeneration pipeline to regenerate plantlets.

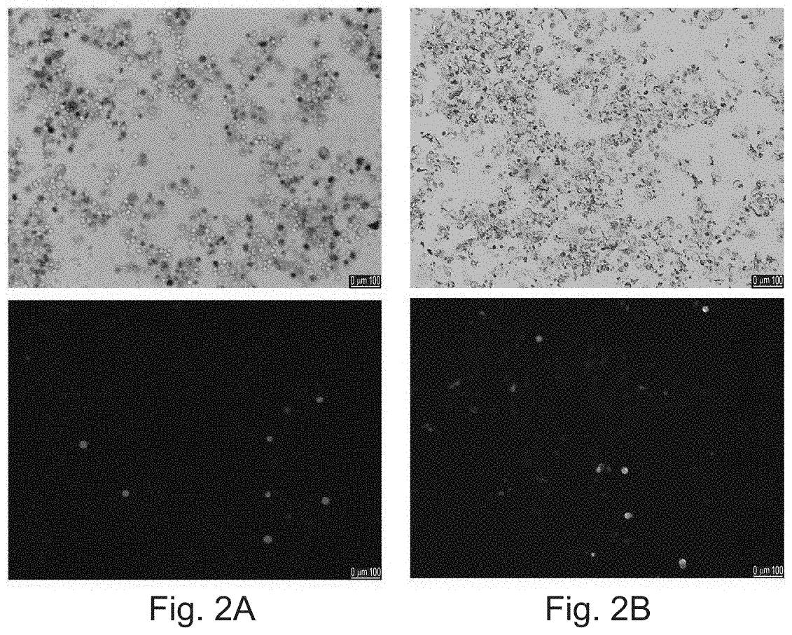

To test whether viable protoplasts from coffee and banana plant material could be recovered, coffee and banana plant material (e.g. calli, cell suspensions) was incubated in a digestion solution for 4-24 h at room temperature with gentle shaking. After digestion, the plant material was washed, filtered and re-suspended in 2 ml of MMG buffer (0.4M mannitol, 15 mM MagC12, 4 mM MES pH 5.6)). Protoplast concentration was determined and adjusted to 1×10⁶. Next, DNA plasmids pDK1202 (carrying a GFP fluorescent marker) or pAC2010 (carrying mCherry as fluorescent marker) were incubated with the protoplasts derived from coffee and banana, respectively, in the presence of polyethylene glycol (PEG). The expression of GFP or mCherry in the protoplasts was detected by fluorescence microscopy 3 days post transfection for coffee (FIG. 2B) and banana (FIG. 2A).

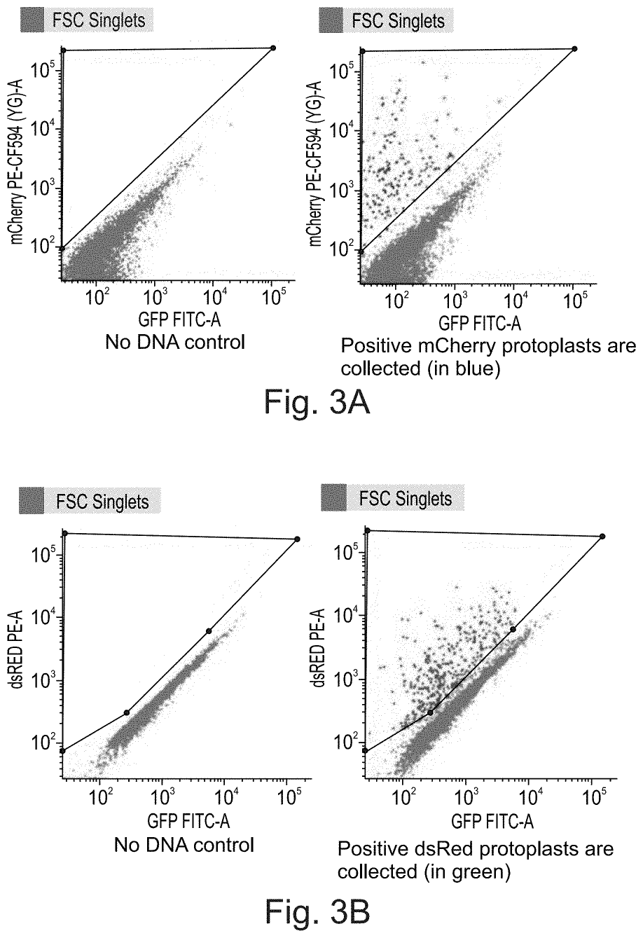

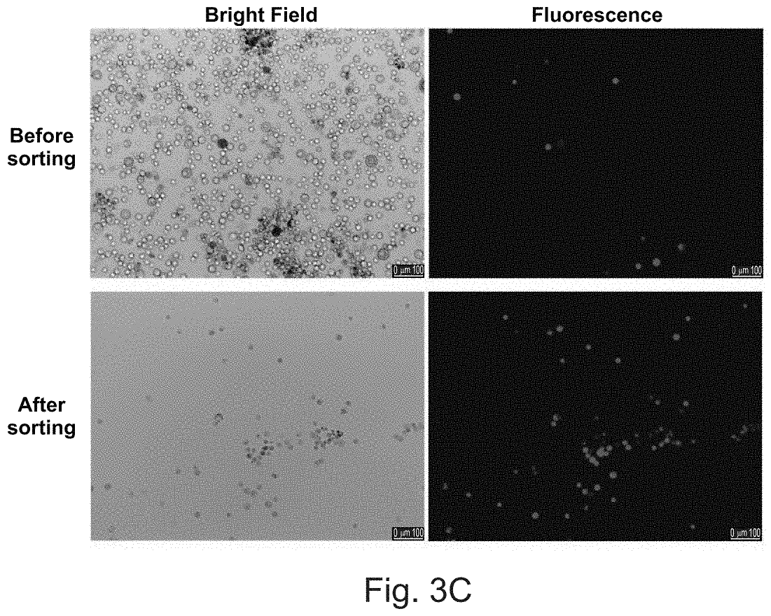

The next step in recovering gene-edited plants was to deliver the CRISPR/Cas9 complex and sgRNAs that target genes of interest in coffee and banana protoplasts and enrich for cells that carry such complex by fluorescence-activated cell sorting (FACS), thereby separating successfully transfected coffee and banana cells that transiently express the fluorescent protein, Cas9 and the sgRNA. Using FACS, positive dsRed or mCherry expressing protoplasts for coffee (FIG. 3B) and banana (FIG. 3A), respectively, were enriched and collected and confirmed that the sorted protoplasts were still intact and indeed expressing the fluorescent marker by fluorescence microscopy (FIG. 3C).

To assess that the CRISPR/Cas9 complex and sgRNAs are functional, 4 reporter-sensor plasmids were prepared that consisted of a red fluorescent marker, Cas9, a GFP fluorescent marker and sgRNAs targeting GFP in one vector. Sensor 1 and 3 have the same sgRNA but different U6 promoters and sensor 2 and 4 have the same sgRNA but different U6 promoters (FIGS. 4A-B). All 4 plasmids were delivered independently into protoplasts derived from Nicotiana benthamiana (FIG. 4A) or Coffea canephora (FIG. 4B) and confirmed Cas9 activity in these protoplasts by measuring the ratio of green versus red protoplasts using FACS. Evidence of genome editing of the GFP marker is shown as a reduction of the green versus red ratio when compared to the control plasmid, which only lacks the sgRNAs. As shown in FIGS. 4A-B, all versions of the reporter-sensor plasmid indicate that Cas9 is active in tobacco (FIG. 4A) and coffee (FIG. 4B) and leads to positive editing thereby specifically reducing the signal of the GFP marker.

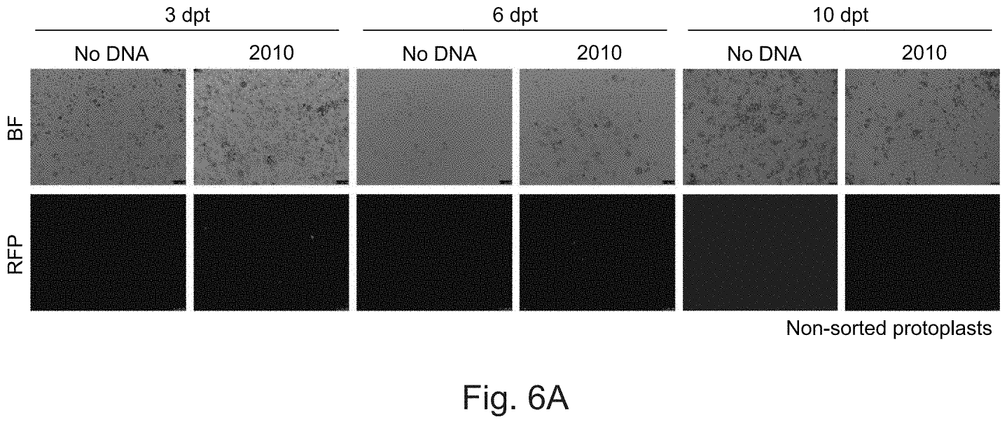

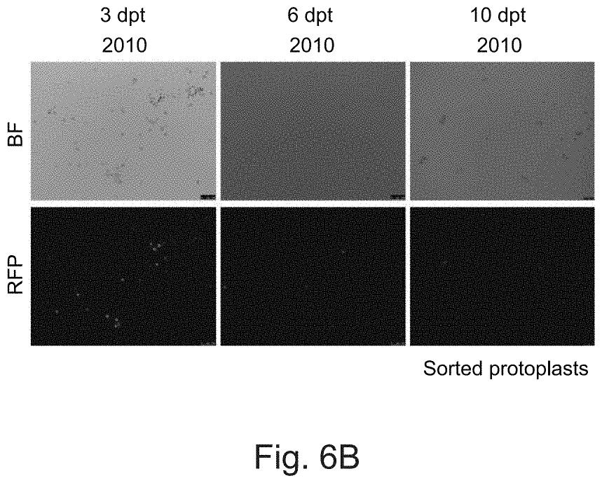

The transient nature of the transfection of the CRISPR/Cas9 complex and sgRNAs that target genes of interest in Musa acuminata protoplasts was next examined. Since all our plasmids consist of a fluorescent marker (e.g. dsRed, mCherry), Cas9, and sgRNAs (under a U6 promoter and targeting an endogenous gene of interest or GFP in the case of the reporter-sensor plasmid), the expression of the fluorescent marker in transfected banana protoplasts was followed over time and the number of mCherry-positive protoplasts was used as a proxy to get an indication of how long the CRISPR/Cas9 complex and sgRNAs might be expressed (FIGS. 5A-C). FACS was used to quantify the percentage of mCherry-positive banana protoplasts over time and set the total number of mCherry-positive banana protoplasts at 3 days post transfection (dpt) as 100%. It was found that already at 10 dpt, mCherry-positive banana protoplasts decreased by 30% of the initial number of mCherry-positive banana protoplasts and by 25 dpt almost 80% of transfected banana protoplasts did not show any fluorescence (FIG. 5C). mCherry expression was also monitored in non-sorted banana protoplasts by microscopy at 3 dpt (FIG. 5A; FIG. 6A), 6 dpt (FIG. 6A) and 10 dpt (FIG. 5B; FIG. 6A), which confirmed that indeed mCherry expression diminishes over time. Moreover, fluorescence microscopy of sorted banana protoplasts shows the progressive reduction in number and intensity of mCherry-positive protoplasts (FIG. 6B) as seen by FACS (FIG. 5C). Taken all together, these results indicate that the expression of vectors carrying the CRISPR/Cas9 complex and sgRNAs is transient and no further Cas9 activity or integration in the plant genome is expected.

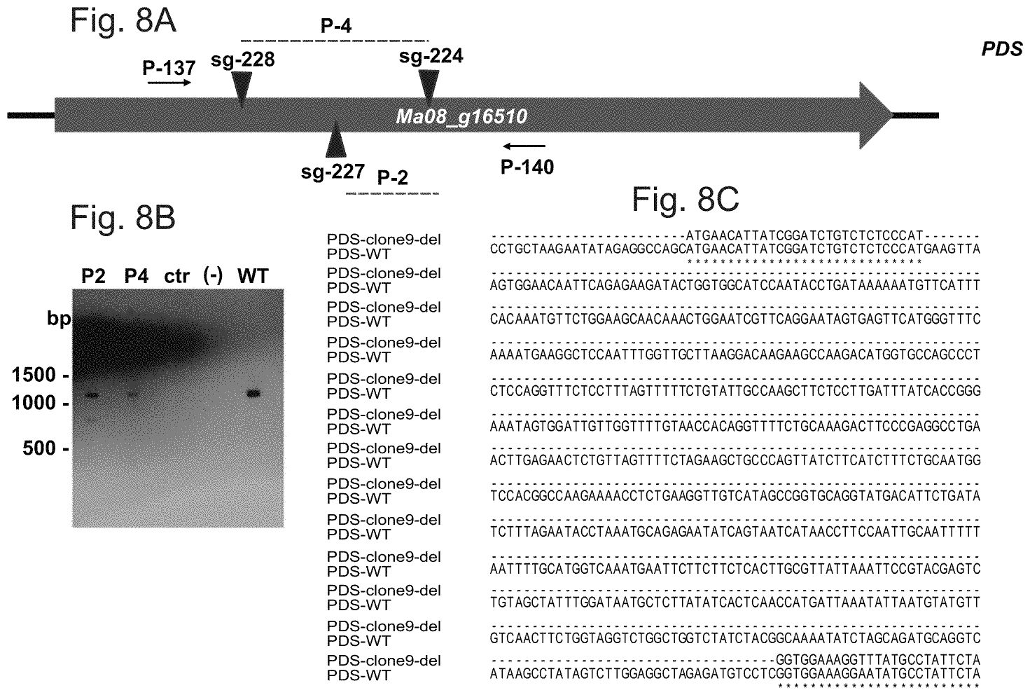

Finally, the above described pipeline for protoplasts isolation, sgRNA design, the system of vectors carrying the CRISPR/Cas9 complex and sgRNAs was used to target an endogenous gene in coffee (FIGS. 7A-B) and banana (FIGS. 8A-C) protoplasts. Annotated PDS genes for coffee (Cc04_g00540) and banana (Ma08_g16510) were used to designed specific sgRNAs as depicted in FIG. 7A and FIG. 8A, respectively. The sgRNAs design was based upon the sgRNA predicted activity and mistmatch identity against the coffee and banana genome to avoid possible off-target genes. After transfections with the plasmids indicated in the figure legends, it was seen that distinct sgRNAs combinations induced indels in both coffee (FIG. 7B) and banana (FIG. 8B; 8C) PDS gene. These results demonstrate that the CRISPR/Cas9 system can successfully be used to introduce precise mutations in an endogenous gene of interest in coffee and banana genomes and that this system combined with the robust pipeline for plant regeneration from protoplasts paves the way to efficiently modify traits of agricultural importance in these crops.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A nucleic acid construct comprising:
(i) a nucleic acid sequence encoding a genome editing agent;
(ii) a nucleic acid sequence encoding a fluorescent reporter which is detectable by fluorescent activated cell sorter (FACS),
said nucleic acid sequence encoding said genome editing agent and said nucleic acid sequence encoding said fluorescent reporter being operatively linked to a plant promoter.

2. The nucleic acid construct of claim 1, wherein each of said nucleic acid sequence encoding said genome editing agent and said nucleic acid sequence encoding said fluorescent reporter being operatively linked to a terminator.

3. The nucleic acid construct of claim 1, wherein said genome editing agent comprises an endonuclease.

4. (canceled)

5. The nucleic acid construct of claim 3, wherein said endonuclease comprises Cas-9.

6. The nucleic acid construct of claim 5, wherein said genome editing agent comprises a nucleic acid agent encoding at least one gRNA operatively linked to a plant promoter.

7-8. (canceled)

9. The nucleic acid construct of claim 1, wherein said plant promoters are identical.

10. The nucleic acid construct of claim 1, wherein said plant promoters are different.

11. The nucleic acid construct of claim 1, wherein said promoters comprise a 35S or a U6 promoter.

12. (canceled)

13. The nucleic acid construct of claim 6, wherein said promoters comprise a U6 promoter operatively linked to said nucleic acid agent encoding at least one gRNA and a 35S promoter operatively linked to said nucleic acid sequence encoding said genome editing agent or said nucleic acid sequence encoding said fluorescent reporter.

14-16. (canceled)

17. A method of selecting cells comprising a genome editing event, the method comprising:
(a) transforming cells of a plant of interest with the nucleic acid construct of claim 1;
(b) selecting transformed cells exhibiting fluorescence emitted by said fluorescent reporter using flow cytometry or imaging; and
(c) culturing said transformed cells comprising said genome editing event by said DNA editing agent for a time sufficient to lose expression of said DNA editing agent so as to obtain cells which comprise a genome editing event generated by said DNA editing agent but lack DNA encoding said DNA editing agent.

18. The method of claim 17 further comprising
validating in said transformed cells loss of expression of said fluorescent reporter and/or said DNA editing agent following step (c).

19. (canceled)

20. The method of claim 18, wherein said validating is by imaging and/or comprises sequencing and/or comprises a structure-selective enzyme that recognizes and cleaves mismatched DNA.

21-23. (canceled)

24. The method of claim 17, wherein step (b) is effected 24-72 hours following step (a).

25. The method of claim 17, wherein step (c) is effected for at least 60-100 days and/or wherein step (c) is effected in the absence of an effective amount of antibiotics.

26-29. (canceled)

30. The method of claim 17, wherein said genome editing event does not comprise an introduction of foreign DNA into a genome of the plant of interest that could not be introduced through traditional breeding.

31-34. (canceled)


SEQUENCE LISTING (74 sequences)

SEQ ID NO: 1 - DNA, 1005 bp, Artificial sequence
  CaMV-35S-promoter
tttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgagaggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctctttcaaggacgacggcacttacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcactgccgacaagcagaagaacggcatcaaggccaacttcaagatccgccacaacgttgaggacggcagcgtgcagctcgccgaccactaccagcagaacacccccatcggcgacggccccgtgctgctgcccgacaaccactacctgagcacccagtccgttctgagcaaagaccccaacgagaagcgcgatcacatggtcctgctggagttcgtgaccgccgccgggatcactctcggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcc

SEQ ID NO: 2 - DNA, 855 bp, Artificial sequence
  NOS terminator
gctcgtccatgccgagagtgatcccggcggcggtcacgaactccagcaggaccatgtgatcgcgcttctcgttggggtctttgctcagaacggactgggtgctcaggtagtggttgtcgggcagcagcacggggccgtcgccgatgggggtgttctgctggtagtggtcggcgagctgcacgctgccgtcctcaacgttgtggcggatcttgaagttggccttgatgccgttcttctgcttgtcggcagtgatatagacgttgtggctgttgaagttgtactccagcttgtgccccaggatgttgccgtcctccttgaagtcgatgcccttcagctcgatgcggttcaccagggtgtcgccctcgaacttcacctcggcgcgggtcttgtaagtgccgtcgtccttgaaagagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgttggtggcatcgccctcgccctcgcctctcacgctgaacttgtggccgtttacgtcgccgtccagctcgaccaggatgggcaccaccccggtgaacagctcctcgcccttgctcaccatggatcctctagagtcgactgtaattgtaaatagtaattgtaatgttgtttgttgtttgttgttgttggtaattgttgaaggatctcaagaagcctgtcctctccaaatgaaatgaacttccttatatagaggaagggtcttgcgaa

SEQ ID NO: 3 - DNA, 215 bp, Artificial sequence
  CaMV-35S terminator
cgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggc

SEQ ID NO: 4 - DNA, 1746 bp, Musa acuminata
atgaacattatcggatctgtctctcccatgaagttaagtggaacaattcagagaagatactggtggcatccaaatcctgataaaaaatgttcatttcacaaatgttctggaagcaacaaactggaatcgttcaggaatagtgagttcatgggtttcaaaatgaaggctccaatttggttgcttaaggacaagaagccaagacatggtgccagccctctccaggttttctgcaaagacttcccgaggcctgaacttgagaacactgttagttttctagaagctgcccagttatcttcatctttctgcaatggtccacggccaagaaaacctctgaaggttgtcatagccggtgcaggtctggctggtctatctacggcaaaatatctagcagatgcaggtcataagcctatagtcttggaggctagagatgtcctgggtggaaaggttgctgcttggaaggacaatgatggagattggtatgagacaggcctccatatattctttggggcatatcccaatatgcagaacttgtttggggaacttggtatcaatgatcgcttgcaatggaaggagcattctatgatttttgcaatgccgaacaagccaggagagtttagcagattcgatttcccagaaactcttcctgcacctttcaatggaatatttgcaatattaagaaatagtgaaatgctgacttggccagagaaagtgagatttgcacttggacttttgccagccatgcttggagggcaagcttatgtggaggcgcaggatgggttgactgttacagagtggatgagaaggcagggtgtgccggaccgagtcaatgatgaagttttcattgccatgtccaaagcactcaactttataaaccccgatgagctttccatgcaatgtgtattaattgctttgaaccgttttcttcaggaaaagcatggttcaaaaatggccttcctagatggtaatcctcctgaaagattatgcaagccaattgttgatcatattgaatcattgggtggagaagtttgggttaattcacgaactcagaaaattgagctaaaccccgatggaactgtaaagcactttttgctcagcagtggaaacataatcagtggagatgtttatgtaattgccactcctgttgatatcttgaagcttcttttaccgcaagagtggaaggatattctgtacttcaagaagttggaaaaattagtgggagtccctgttatcaatgtacatatatggtttgacagaaaactgaagaacacctatgaccatcttctattcagcaggagtcctcttttgagtgtatatgcagacatgtccgtcacatgcaaggaatattatgatcctgatcgttcaatgttggaattagtgtttgctcctgcagaacaatggatctcatgcagtgaccaggaaattgttgatgccactatgcaagaactggctaagctatttcctgatgagattgcggcggatcaaagtaaagccaaaattctgaaatatcatgtagtaaagactccaagatctgtttacaagactgttccagattgtgaaccatgccgccctttgcaaagatccccggttaaaggtttctatctggctggcgactatacaaaacagaaatatttggcttccatggagggtgctgtgctatctgggaagctttgtgctcaggcaatcacacaggactatgatgtgttggttgctcaggccgcccagagagaagtccaggtgtcaatatga

SEQ ID NO: 5 - DNA, 1746 bp, Musa acuminata; identical to SEQ ID NO: 4

SEQ ID NO: 6 - DNA, 2256 bp, Musa acuminata
atggcctcgctcaccaccatcatctacaagtcctcctccccctgctcttcctcctcctcccctccatgttcgcccaccatcactactagttcaccgcgcttgcagtgccctccccccccccacccgtcatctgctccttccatggctctctccgcattctccttcccctgccatttcctcggcgcagctccctccttcactgatctccaacaccagcagcccctgcccacaagagttctcaagccgaagaaaagggcctgtgtttgtgcatcgctatcagagaccggggagtatcactcacagagaccgccaactccactcctcgacaccgtcaacttccccatccacatgaagaatctctcggtccgggagctgaagcaactcgccgacgagctccgctctgatatcatcttcaacgtgtctaggaccggcggtcacctcggttccagcctcggcgtggtcgagctcaccgtcgcgctccactacgtcttcaacgctccgcaggacaagatcctttgggatgtcggccaccagtcgtatcctcacaagatattgacgggaaggagagacaagatggcgacaatgaggcagacgaatggcttgtccgggttcaccaagcggtcggagagcgagtacgactgcttcggtgccggccacagctcgaccagcatatcggcagccctcgggatggcagtcggaagggatctgaaggggcgaaagaacaacgtagtggcagtgattggggacggagccatgaccgcggggcaagcttatgaggccatgaacaatgctggctatctcgactccgacatgattgtgatcttgaatgacaacaagcaggtctctctgcccactgcaactcttgatggccctgttcctccagttggagctctgagcagtgcccttagcagactgcagtcctccaagccactcagggaactgagggaggtcgctaagggagtcacgaagcagatcggtggatccatgcacgaaatagctgccaaagtcgacgaatacgctcgaggaatgatcggtggatcagggtcgaccttgttcgaagagctcggtctctactacatcggtcctgtcgatgggcacaacatagatgacctggtcgccattctcaaggacgtgaagagcaccaagacgacaggccctgttctcatccatgtcgtgaccgagaagggacgagggtatccctacgccgagaaagctgcagacaagtatcatggtgtcgccaaattcgatccagcgacagggaagcaattcaaatcgggctccaagacgcagtcttacacgaactacttcgcggaggcgttgattgccgaggcggaggtggacgaaggcatcgtcgcgatccacgcggccatgggaggaggaacagggctcaactacttccttcgctgctacccgacgaggtgcttcgacgtggggatcgcggagcagcacgcggtcacgtttgcggcagggctcgcctgcgaaggcctcaagccattctgcgcgatctactcgtcgttcctgcagcgggcttacgaccaggtgatacacgacgtggacttgcagaagctgccggtgaggtttgcgatggatcgggcgggactcgtcggagcggacgggccgactcactgcggctccttcgatgtcacctacatggcttgcctaccgaacatggtggtcatggcgccctccgacgaagcggagctgttccacatggtggccaccgcggcggccatcgacgaccggccgtcctgcttccggtaccccaggggcaacggcatcggtgttccgcttccccccggaaacaagggtattccacttgaggtggggaaggggaggatactgaaggaaggggagagggtgactcttctgggatacggaacagcagttcaaagctgcttggccgcggcatcgctgctggaggaacgcggcctaaagatcaccgtcgccgacgcacggttctgcaagccactcgaccggagcctgatccgaaacctggcgaggtcgcacgaggtgctcctcaccgtggaagaaggatccatcggcggtttcggctcccacgtcgtccagttcttggccctcgacggcctcctcgacggcaccctcaagtggcggccggtggttctcccggatcggtacatcgaccatggatcgccgcgcgatcagctggcggaagctggattgacgccgtctcatatcgcagcgactgtgctcaacatcctcggacagacgcgagaggcactcgagatcatgtcttag

SEQ ID NO: 7 - DNA, 2157 bp, Musa acuminata
atggctgcatccacgcttcccttctcttgccatttgcctgctctgctttcctcggatctgcagaaggcttcccccctcctgcctacgcagttgtttgcagggactgatctcccgcaccaccggcatcgtcatgggtttctcacgcctaggagacggtcatgtgtttgcgcctcactatcaggaactggggagtacttctcgcagcggccaccaactccgctgctggacaccgtcaactatcccatccatatgaagaatctctcggtcaaggaactcaaacaacttgcggacgaacttcggtcagatgtcatcttccatgtctctaagacgggaggacatcttggttcgagccttggagtggttgagctaaccgtcgctctacactatgtcttcaatgctcctcaagacaagatactatgggatgttgggcaccagtcgtacccacacaagatactaacagggaggagagacaagatgcctacgttacgacggacgaatggattatctgggttcacaaaacgatcagagagtgactatgatagctttggaactggtcatagttcaaccagcatctcagcagcccttgggatggctgtcggaagggatctgaagggcagaaagaataatgttatagcagtgataggggatggggccatgactgctggacaagcatatgaagctatgaacaatgctgggtatcttgactcggacatgattgtcattctgaatgacaacaagcaggtctctctgcccactgcaagtcttgacgggcctataccaccagttggagctttaagcagtgctctcagtagattacaatctagcagaccattaagagaactgagagaggtcgccaagggagttacgaagcagattggtggatcgatgcatcaaattgcggcaaaagtcgatgaatatgctcgaggaatgattagtggatctggctcaactttgtttgaagagcttggtctctattatattggcccggtggatggccacaacatagatgacctcgtttccatactcaaggaggttaaggacacaaagacaacaggtccagttcttatacatgttgtaacagaaaaaggacggggatatccctatgcagagagagctgctgacaagtatcatggtgttaccaaatttgatccggccactgggaaacaattgaagtcgatctctcagactcaatcttataccaattattttgctgaagctttgatagctgaggcagaggtagacaaagatatagtcgcaattcatgcagccatgggaggtggaaccggccttaactacttccttcgtcgatttccaacaagatgttttgatgtcggtatagccgagcagcatgctgttacatttgcagctggtctagcctgcgaaggcctcaagccattctgtgcaatctactcatctttcttgcaacgggcttacgatcaggtgatacatgatgtggacttgcagaaacttcctgtaagatttgctatggaccgagcggggcttgtcggagctgatgggccaactcattgtggtgcatttgatgtcacatacatggcatgtctgcctaatatgattgtcatggctccttccgatgaagctgaactgtttcacatggttgccactgcagcagccatcaatgaccggccatcctgcttccgatatccaagaggaaatggcattggcgttcccctgccccaaggaaacaaaggtgttccgcttgagatcggcaaaggcaggatattgattgagggtgagagggtggctcttcttggatatggaacagcagttcagagctgtgtggctgcagcttccctcctggaacaacgtggtctaagggtcacagtggctgatgcacgattctgcaagccgctggatcatgctttgattcggaacttatctaaatctcaccaagtgctgattacagttgaagaaggatccatcggagggtttggctctcatgtcgcccagttcatggcacttaatggtcttcttgatggcacgataaagtggagaccgctggttcttcctgatcgttacatcgagcatggatcacccaatgatcagctggcagaagctggtttgacaccgtctcatgttgcagccacagtgctcaacatccttggacaaactagagaggcacttgaaatcatgtcatag

SEQ ID NO: 8 - DNA, 2913 bp, Musa acuminata
atggctacttcttccatttccagaccctcttcgaagctctccaagtccccatcccgatcccataacccctccaattcctcctcttcttccaaatcccaatcttcttcctccctttcctcccatcttgcaatggtggaactcaaatcgcgggtcctgtcggcgctgtcgaagctttccgaccgcgacacccaccagatcgcggtcgacgacctggagaagatcatccggaccctccccgccgacggcgtccccatgctcctccacgccctcatccacgacccctccatgccctcgcccagcccccaggacccgcccgggtccaagaacccctccttcctcgtgggtcgccgcgagtccctccgcctcctcgcgctcctctgcgcctcccacaccgacgccgcttccgcgcacctccccaggatcatggcccacatcgtccgccgcctcaaggaccccgcctccgactcctccgttcgcgacgcctgccgtgacgccgccggttcgctcgccgcgctctatctccgcccctcgctcgcagcggcggccgctcatgtggacggcgctggcagcggaggaccgtctccggtggtggcgttgttcgtgaagccattgtttgaggccatgggggagcagaataaggcggtgcagggcggggctgccatgtgcctcgcgaaggtggtcgagtctgctggaggtggcggcgtcggcggtggtgggcaaagggaggagggaagggtgatgacgacaggagtggttttccagaagttgtgccctaggatctgtaagctgcttggtggccagagctttctagctaaaggagcattgctttcagtcatctctagccttgctcaggtaggagcaatcagtcctcagagcatgcaacaagtgctgcaaactattcgtgaatgtcttgagaatagtgactgggctacccgtaaggcagctgctgatacactctgtgtgttggcctctcactcgagccatgttcttggtgatggggctacagcaaccataactgctcttgaggcctgccgttttgataaggtaaaacctgttagagatagcatgatggaggcactgcagctatggaagaagattagaggagatggaactttggcagacacaaaagattctagaagctcggacttaactgataatgaagaaaaggaagatcataaaaggtttaaccctagcaaaaagttagaatctttaaaaatttcatctgctggattttcatctggtgaaagtgactctgtctccaaagaaaatggcaccaacatgctagagaaagcaacagtgcttttaatgaaaaaagcaccatcattaaccgataaggagttgaatccagaattcttccaaaagctagagaagaggagtttggatgactttcctgttgaagtggtgctacctcgtaggtgcttacagtcttcccattctcaatgtgaagaaggatcagaagtaacttgtaatgattcgacgggcacatcaaactgtgatggagcagcactccaggaatcagatgacactcatggatataacactgccaattaccggaatgaagataaacgaccagggccttacaagaaggtgcaggacttggataattttgctcgggacaaatggacagagcaaaggggatctaaggcaaaagaatcaaaagcaaaagttttgaatgttgaggacacaactgaagtctgtcagaaagatccttctcctggtcgtacaaatgtccctagatctgatgccaacactgatgggccttttatgagcaatagggcgaattggactgcgatacagaggcagttggctcaattagagaggcaacaagccagtctcatgaatatgttacaggacttcattggtggctcccatgatagtatggtaactctagaaaatagagttaggggtcttgagagagttgttgaagaaatggctcatgatttggctatgtcatctggaaggagagttggaaatatgatgctgggatttgacaaatctccaggaaggtcttcaagcaagtacaatggccttcatgattactccagctcaaagtttggcagagttggtgaaaggtttcacttgtcagacggtttggtaactggtgttcggggaagagattctccgtggaggtcggaatctgaagcatgggattcctatggatatgtagcttcaagaaatggtgttatgaacactaggagagggtttggtgctgttccggtggatggtaggttacacaaaaccgagcatgatactgatcaagtcagtggtaggcgggcttggaacaaaggaccaggaccgtttaggcttggtgaagggccttctgcaagaagcgtttggcaagcctcaaaggatgaggctacacttgaagctatcagagtagctggggaagacaatggaacatccagaaatgcagcacgagtagctgtaccagaattagatgctgaagctttaacagatgataatccagggcccgacaagggtccactttgggcgtcttggactcgtgccatggattcacttcatgttggtgacattgattcagcttatgaagagattctatctactggtgatgacttattacttgtaaagctaatggataaatcaggtccagttttcgaccagctctctggtgaaatagcaagtgaagtcttgcacgcagttgggcaatttattctggagcaaagcttgtttgatatagcattgaattggcttcaacagttgtcagatcttgttgtagagaatggagccgacttccttagagtccccctcgaatggaagagagagattttgttaaatcttcatgaagcttctgcacttgaactaccagaggattgggagggggcagcaccagaccaattaatgatgcatttagcatcagcctggggtctcaacttgcaacagcttgtcaagtag

SEQ ID NO: 9 - DNA, 2898 bp, Musa acuminata
atggctacttccacctccaaaccctcttctaggctctccaaaccctcttcctcctcttccaaatcccaatcttgctcttcctcctcttctggcctttcctcccatgtcgccatggtggagctcaagtcgcggatcctcgcggcgctcgcgaagctatccgatcgcgacacccaccagatcgccgtcgacgacctcgagaagatcatccgcaccctccccgccgagggcgtccccgtgctcctcaacgccctcgtccacgacccctccctgccttcgcccaccccccaagaaacccccggctccaagcacccctccttcctgatcgctcgccgcgagtccctccgcctcctcgccctcctctgtgccgtccacactgacgccgcctccgcccacctttccaagatcatggtccacattgcccgccgcatcaaggactcggcctctgactcctctgttcgcgatgcctgccgcgacgccgcgggctcgctcgcggcgctctaccttcgcccctgggtcgcggcagcggctgcgccggaggatagcgctggcggcatcggagggtcatcttcgatggtggcgctgttcgtgaagccgctgttcgacgccatgggggagcagaataaggcggtgcaaggcggggcagccatgtgccttgctagggtggtggagtgtgccggggctaacgatgatggtggggagggggaggagggaagggtgacggcgtcggggacgatgctccagaggttgtgccccaggatctgtaaacttcttggaggccagagctttcttgccaagggggcgttgctttcagttgtctctagcttggcgcaggtaggagcgatacatctgcagagcatgcaacaactgctgcaaattgttcgtgaatgtcttgaaagcagtgaatgggctacccgtaaggcagctgcagacacattgtgtgtcttggcctctcactcgagtcatttgcttggtgatggagctgcagcaacaataactgctcttgacgcttgccgttttgataaggtaaaacctgtcagagatagcatgatggaggcactgcagctatggaagaagatcaaaggacaaggagagggtggaacatcaggagacaagaaagattctagaaactctgacttaactgatagtgaggaaaaggcaactcacaagaggtccaactctaataagaggtcagaaactttgaaaaactcatctgctggttcttcacccagtgaaaatgattctgtatccagaggaaaaggcactaatatgcctgagaaagcagtcatactgttaaagaaaaaagcaccatctttgactgacaaagaattgaacccagacttcttccaaaagcttgagaagaagagttcagatgacctgccagtagaagtagtgttacctcgtaactgtttgcagtcttcccattcacaatgtgaagaaggaccagaagcaatttatagtgattcaacggaaacaccaaagcatagtggagcaacactccagcaatcggatgacattcatggacataataatgctaattatcataatgcagagaaacgactgggggttcacaataatgtgcaagactcggattattttcctagggggagatggatagagcaaagaggtatcagagcaaaagaatcaaaagcagaggattttgatggtgacgatagattggaggtctgtcagaaagatccctctcctggctgtcttaatgtccctagatctgatgctcatgctgaagggtcctttatgagcaataaagcgaattggtctgccatacagaggcagctagcccaattagagaggcaacaaatcagtcttatgaacatgttacaggactttatgggaggttcccatgatagcatggtaactctagaaaatcgagtgaggggtcttgagagagttgttgatgaaatggcccgtgatttggctattaaaccaggaaggagaggtggaaatatgatgcagggattcgataaatctccaggtaggtcttcaggcaagtacgatggccttcatgattgctccaactcaaagtttggcagggacagtgaggggcggttcccatttccagagaggtttctctcatcagaaagtatggtttctggagtaaggagacgaggttctccttggaggtcagaatctgaaacatgggattaccatggtgcctcaaggaatggtgtcgtgaactctaggagagggttcaatgctgttccagtggatggtagagtacctagatctgagcatgacgctgatcaagttggtggcaggtgggcctgggataagggaccaggaccatttaggcttggtgaagggccttctgcaagaagtgtttggcaagcctcaaaggatgaggctactttagaagctatccgagtagctggggaagacaacataacatccataactgcagcacgagtagctgttcctgaattagatgctgaaggtatagcagatgataatctggggctggacaagggtccactttgggcttcgtggactcgtgcgatggattcactttatgttggcgatgttgattcagcttatgcagagattctgtctactggtgatgacttattacttgtaaagctaatggataaatctggtccagtatttgatcagctctctaatgaaatagcgagcgaagtctttcgtgcaattggacagtttgttctggaagaaagcttgtttgatatagcgcttagctggctccatcagttatcggatcttgtcgtggagaatggaagcgagtttctcagcatccccctcgaatggaagagagagatgttgctgaatcttcgtgaagcttctgtttcagaaccaccagaatattgggaggggacaccaccggatcagctaatgatgcatttagcggctgcatggggtctcaactag

SEQ ID NO: 10 - DNA, 23 bp, Artificial sequence
  sgRNA sequence
gtctctcccatgaagttaagtgg

SEQ ID NO: 11 - DNA, 23 bp, Artificial sequence
  sgRNA sequence
gttcaggaatagtgagttcatgg

SEQ ID NO: 12 - DNA, 23 bp, Artificial sequence
  sgRNA sequence
agagggctggcaccatgtcttgg

SEQ ID NO: 13 - DNA, 23 bp, Artificial sequence
  sgRNA sequence
acggccaagaaaacctctgaagg

SEQ ID NO: 14 - DNA, 23 bp, Artificial sequence
  sgRNA sequence
ggtgggcgaacatggaggggagg

SEQ ID NO: 15 - DNA, 23 bp, Artificial sequence
  sgRNA sequence
gggggagggcactgcaagcgcgg

SEQ ID NO: 16 - DNA, 23 bp, Artificial sequence
  sgRNA sequence
ggaaggagcagatgacgggtggg

SEQ ID NO: 17 - DNA, 23 bp, Artificial sequence
  sgRNA sequence
acggtgtcgaggagtggagttgg

SEQ ID NO: 18 - DNA, 23 bp, Artificial sequence
  sgRNA sequence
agatccgaggaaagcagagcagg

SEQ ID NO: 19 - DNA, 23 bp, Artificial sequence
  sgRNA sequence
aaacaactgcgtaggcaggaggg

SEQ ID NO: 20 - DNA, 23 bp, Artificial sequence
  sgRNA sequence
cgcctcactatcaggaactgggg

SEQ ID NO: 21 - DNA, 23 bp, Artificial sequence
  sgRNA sequence
gtcctcccgtcttagagacatgg

SEQ ID NO: 22 - DNA, 23 bp, Artificial sequence
  sgRNA sequence
tggggacgccgtcggcggggagg

SEQ ID NO: 23 - DNA, 23 bp, Artificial sequence
  sgRNA sequence
gcgagggcatggaggggtcgtgg

SEQ ID NO: 24 - DNA, 23 bp, Artificial sequence
  sgRNA sequence
aaggaggggttcttggacccggg

SEQ ID NO: 25 - DNA, 8545 bp, Coffea canephora
aattatgatgatgatgaagatgattcattgaggatattagacgtaaatggatgtgtaaattggatcttcgcctaatgctgatgataaatttggtttggtggtgcattggataggataggataattttagtggtccaacaaggagtaatattaatggtggctgggtagcagatcagaattatgagttagagagggctaactgctagcgtattgctaccattcaagaaaatagtgagggagaatgaatgaatgatgacgtacactactactaccactacaactactgctcatggaactactgtgaggacaatgacagggcccggtgccgaatgaaaagtgcagagagagagagaggcaggaaacagaaggaaaatggatggacggaggcggagcctggtggagctttggcacaaaggtaaactacagtggaaggtgaaaagtaagttccttcctcgtgtaagtgaagtaaaagatggatagaatattctaagccataacaaatgtgtcccaataacaaatgcggccaaaacccaccaaattacatcacgcttccctcgcaaaaccattgctatataataattattacactactgcctttcgcatttccctttttatcttttcccttgtcacctcttgtgggtatttttgtgcgtatccagtcagtggtagttaactgctatacctcctagctgcaacaggaaggaggatttctgatggcctttactctgcaatcctgcctgccttcctttcttgcttctcccttatctcactctgaaagaactcgcagctaaaaaggagtttccttggactattctttgctcgcctagaggtaatcaagcttaccacctcaaactatagtctttgtagtttgtactgggaattttgcacctttcttttccaccgtcaattccagttctttttgggttaaattttgcagctgctcaaaatttgagacgctcaagtctttgctactctgtctatttatttcttgttggtttacttgatttgcttcttttccttgtcatgatatctgatacccttataactgtgtgggttaagtcatttcctgtatagctgtttcgtggctacatgtatggaggagagttgttggctgttgcttttttttttttggcccgtgtgggggtggggggccgaggaatgttacctaattatagtcagcacagcttaatctcttggttttaattgtattaatgaaccatttgatttaggaaaagttccaaattgattgcactgtgacgttggtccgtttagaagtctaaaagcaaactcaattttgcgcccaatttggagaaatgtctcaacttggacatgtttctgcactaagcctgaccaggcaaactagtgtgattaatgttcggagccctcattctgcttggaagtgtggcctttgttttggttctgggcaaatgacctcactttcatttggaggtggtgattctatgggagataaattgaaagttcaagttgcaaattcagttgtcgtgagatcaagggcggaggatgcaggtcctttaaaggtatgcttctgaaaaaatgtatctgatgatcatcgatatcaaggacaacaaacaataacaaaagaagggaaactaatccaatttactctttgcttcatcatgcaggtagcttgtattgactatccaaggccagagcttgaaaatgccgtcaactatttggaagctgcttatttatcatcaacattccgtacttctcctcatccaaataaaccattagaggtggtgatcgccggtgcaggtggaaatatcacactcaatctttaattatatttttctgccattttatttcgaaagtaaatcttatttccagtgaactcataagctgtgctatggtatccatttagattatagtttttcacttttcaaacatgtctcgttttagtattattccatgcttttggttcataagctctggcagccacacactcgcttttgtagctaagaacagtgtttaataatttttggcagaataatttgacttcattatgcatgagatttcctatccactttcctccacataatttaggtgctcctcatgattggttaaactctgaaaggtttcgtcactgtacatgcataggtaggcttgtgaatgaatttggggctgtcttatttaggagtcctatcagatgattatctggtttgcaagacggatcactttttatagctgatattttatatgttttagcctccattagaacctatgttgtcttattttggtattttgtcataaatttgtatcatcggatgttataagtcaattgcttctgaaaataagtcaaggtatgacatacagaacaaagtctgttatgaaataaatttccacttacttgattaagttttatactttcaggtttggctggtttgtctactgcaaagtatttggccgatgcaggtcataaacctatagtgttggaagctagggatgttctgggaggaaaggtagccaaattattactcattagtgttcatgaattccttgtggcataatggactgtgtcaaagttcaaggaaagtctttcaaaattttccagtatatggatgtgggagttggtctatatgtgtgcataatgtgtaaactttttgatatccaagtttctgtatgtgcattgcacacagtgttatattggtaaaatctgtggttggtatgttaagggaagatagaacaatattgttgcaattattggttgacttctaaaactagcttccatcatttacttatgcaaaattgatgtgtagaggaatatgatctattaacctctttatctaaggaatacttttcctcttctgaaaattatttgtctgtacctaggttgctgcatggaaagatgatgatggagactggtatgagactggcctgcacatattctgtaagtataaggaagaaatgtaacgatttacttaaaccttgtaatgatgactgctactggaaggattgctttaatcatgctcttttcaaatgctctcttgcccatattgtcctctggaaaactgttagtctttgatattaaggcaagctatgctgatctcttataagttttataattcttatggagacatctcttcttttttgtgaaattacattggatttttctaaatttttctaatccaactttactgctgttaggataacaaagggtacgaaccacggtacttaaacatttacttaaacattgttgagcaaatatcttacaagttgcaccaggttagcattaatggacaacattgtcttcttctcagtaaaatcagttaaggttcttggaaaggtgattaatcgtaaagaggttattttaattgacctccaaatatcatgggatgttgttttgtcaaattttcttgattttcgtatttgccttatcttgttccgtgcttttttgaatttcttatgagcatgaatttagatgattcttcttgtttctttttaagatacattatgatgcagcaaataacttgtgacattgattcttgatccaccttaagttggggcttacccaaatatgcagaacctgtttggagaactaggaattaatgatcggttgcagtggaaggagcattcaatgatatttgcaatgccaaataagcctggagagttcagtcgatttgattttcctgaggtgctaccagcaccattaaatggtgagctaatttgtgcagccaaatttcaaatgaagtaacttgtttttatgtggatattgtgttcaaattggtcttgcaggaatatgggccatcttgaagaataatgacatgcttacttggccagagaaagtcaaatttgcaattggactcttgccagcaattctgggtggacaatcttatgttgaggcacaagatggtataactgtcaaagactggatgagaaagcaagtatgcaaccattttcagtagaatgataagttagcaagtttaacaacccactactatgccaagttaatgcttacctaagcttcactacaaagatgaacttttctttcctttctgtatttcctttgcttccgttgagaagttgtattagtgcatttttctagaagaatatggtctaatctttgactgtattttagggcataccagatcgggtgactgatgaagtattctttgccatgtcaaaggcactgaacttcataaatccagatgaactttcaatgcagtgcattttaatagctttgaaccgatttcttcaggttggatccattcctctttctgtgtctctgtgtgtgtgtttttgataacatctctaacttatagtgagatgctaggattttcattcaaataatcacgtaaataaaatgtatcacctgcatttaatagacttcctcatgcagtatatacaaattgaatgacttacttttgcatgtagtggacatttcttactcactctatgaccaaggaagatcacttattttcatttgttaaaaccaggtcccattgcctaatgccatgaatcttccatctatagtgaaattttttatccacaattgagcatttctttttgggataaattttttaaagtccaggcctttattctgtagtgcccttcgtactgctccaacacacagagcaacactaagaaacagtagtctctgtgcagttcattgctgttctttagttccttgtttctttttttttttccttgaccagaaaattgaaagcaggttaattacctacagtctgaacatatagatctcttgagcacacaggagtacatgcaatgtctttaaggagtaggactttatggattgaagtttctcaatctttagaaggcagatggattagttttttttttttttgacaaaaaaaagagaaaagatagattatgtttttagggttttgaagttttctttaaggcacggggtgctttgcagttcttaatctacttctggcttcctttacaatttatacctccgttttcttaataaagttcttgccactttcatatgtaaattagaaggatgtgatagagatttctttctatcgtattagctgtttgaaagaattttagaatcgataaacaggagaagcatggatccaaaatggcatttttagatggtaaccctccagagagactttgcatgccgattgttgagcacattgagtcacgaggaggcagagtacaccttaactcaagaattcagaaaattgagctcaatgatgccggaagtgttgaaaacttcttgctgagtaatggaactgtgattagaggagatgcttatgtatttgccactccaggtagagtctttattaatctaagaaatcatacatgttccccagttttttgtgaactatcttaagattgctagtttgatgtgacgataacagttgatatcctgaagcttcttttgcctgaggattggaaagagatgccatacttcagaaagttggagaaattagttggagttcctgttataaatgtgcacatatggttagtgatttagttttcagcaattctaaagatattactcaacagttgtcctttttgctataaaggttttatctagatgattatttctaatatatacatttacattatgcgatataaaactacttaaagttcatcataatatacaaagtgtatgacctttaaaggataagtttgacctgcaaagatgagtgctattttgtggtcgaaatgatgcaattgactatccttgttggtaaaatcttcactagttatgaattaacacctgatatgctttctgtatcatttcaaaatgacaatctgttcctaacgttcattggattaatcaggagtaagattttatggattcctcctgtaactacacaaaataacacttagaatatggttccctacaggaatatcatcttgtataagtgaacaatcctatttgttgtcacaaattgcaataatatcttagctcagtgatattgatataattgacttcaattgcaggtttgacaggaagctcaggaacacatatgatcatcttctttttagcaggtcttttccatactcgtaccaccagtgaacaaaattttattctgtattcctatctttgaatgtttttgtcttaacagatctcttaacacaaaatcagaacaactatgcttacactatctgcaatttggaaaaatatagtgtcttaagatcttatatgcattactctaatgtgttgattttctgttactgaaacaatgaagcataagacaatttgaaccattttgtgtacaatcatgagttgttttttcctttttccctgttccctaatggggcttgaagagggaaaagtaacattgccccagtttcaagtcccatcctatgctatttgacttgtttcctgaaccaaccttctttctcttgcagaagtccacttcttagtgtgtatgctgacatgtctgtgacgtgtaaggtattccctgtacactgtttaagactcataatgtaatatacttgtattggctctcaatttaggtttttttttccttcctcccatcagcaaggcagcaaagtcatttgcttaaaatttccaaatcacatgacagaaatcttattttgtgcatggatgtaaggtatattatactgaaaaataagcaagttggcatactcaccatgtaatagtttagagaaagaaagtccgagtatgacccagagttcttttcaggcaggtaccctagagttaaatcattgggctaaagcaaattctactcaaagtcaaaaattcatctcaaattgttggaagcttttagcgcatctaaacagtttcagttagaaactggttgctattaattattctagcctctctttatttatttgtatatcggtggttgggaagttgtatctttgggctgcaacttgatatgatttgttcacaacaatttgtgatgactatggtcagaggagctatctttaagctacccttaaacacaaaagtaaaatttatgcaggaatattacagtccaaaccaatcaatgttggagctagtttttgcacctgcagaagaatggatatcacgaagtgatgaggaaattattgatgctacaatgaaggaacttgcaaaattatttcctgatgaaattgctgctgatcagagcaaagcaaaactcttgaaataccatattgtaaaaactccaaggtgacttttttgtctttctattccttgctattatagaaaattggaaacaatgatataatacgttttgctcaagtccgctggaatgttgagaatgtgaacggtcctctttgtaatggtaatgcgctggatcatgtccatgaaatatagctttgtagcaaaatcttttcataacaatttggctcactgtacctcaaaattcattttatgccttgtcaacctataaagcacctgaaatttgaatttcattgagattcagaattctccagtcattttattattggcctctgaaaatgaaaatggagcttttctttttctaggtcagtttataaaactgtgcccggaactgaaccctctcgtccgttgcaaagatctccagttcaagggttctatttagctggtgactatacaaaacagaagtatttggcttcaatggaaggtgccgttctttcaggaaagctttgtgcacaggcaattgtacaggtgatatttcactggtccaatatatacctgcagtgatgcacacactgttgtatggcatgatagagtacctacatcatgcaaattttaggttatgctgtgatatctgcagcttgaggtagtcagataattattatgctctatctagagttcaaagcatcagggtgtgtgactcgggatattgaacatcccatcccccttgttttatacaacttacctacatcaggcctgaggaagccaccaagtcaaccaccattatgaattacctttgccttggccattgttacagtcaaatttgtgacattcggatcgaggaagtgaggtggttttctagtaatctctggagaaaggaatatcaagcacgatcaacagttccagcagaactaaaatcctgaatatgattgaatattgcacaaatgcttgcttactgctatctgtctggtggggatgggcttgtttcatctatatggcgtggttaacatatttttcgttctagcataatcgagaggaagcttatgaagtgcctgaattttgtgaattgactactagaaattaatggtgtttggaggggagtatcgaaacatggagcagaagcaaagaatggaagaaagggatgccttgctgctttaaattaatatgcttttctgtctctctctgccgaccttttaaaccatgcaataactgtgtgttttgcaggatagtgagctgcttcttgccggcattgagaagagggtacccgaggcaagcacagcctgacaaacacaaagctgattactgggaaaagtggataggtgactggggcaggctgataatatatatatcacaaattagattcaaccctgtgcgaatgcacaggccattgtcttcatttggaagctgtgtcataaaataaaacaagtcattcttataattttctctctataaatacaacttttgcatctttacc

SEQ ID NO: 26 - DNA, 1611 bp, Coffea canephora
cccagaatccaccatttcagcacgatcatttgcagttcttgccttctaatttcacacacagcacacactcttcatccagtcagagctcattgcttctttcttttgccattcttaccttatatagcagcagtgaaaccagaactgatccctggagctggaatcatatcttgggttgcttttcttgtcaacccttttgttcatttttattgggttttcaagcttaggcctataaagtgtatgcatcatggctttaagtacatttgcgttccctgcaaatttaagcggggcagtcgtctcagattccataaagcggagtcttctctattctagctggctctatgggacagatcagcatcttcattttcaatccatgaataaccaggtttgacatctttctgatgatttagctcaaaataaaatctttacaaaactatcattgaatggatatcctgatcgccccttttggataaaaactagtttttgattccttctcacctgatcacgccgttgaacttgtatttgtgcgctttttcgtttgtgtttgttttgtcggtactgataactttgtgcttaccattgctgttattaaattgacattgaattatctgttgttcttcttgcattttgttgagaggtactgtgtttatcattacatatcttagtattggacttcttgaatgaaacgttggttttggactcattttgttgtaaaggtcacaaaaaagtccagtggagttcgggcatcactgtcagaaagaggggagtattactcgcagagaccaccaactcctctactggacactatcaattatccaattcacatgaaaaatctttctactaaggtgactacatgtttgatgaagttgtgtaataatgattgcttgtaatgtatattattaactgtctgaaatttaaagcaatttcttgattcaggaattgaaacaacttgcagatgaactgcgttcagatatcatttttaatgtttcaaagactggaggtcatcttggttcgagcctcggtgttgttgagttaactgtggctcttcattacttattcaactgcccccaagataagatactttgggatgttggtcatcaggtaatgatttaaacttgatggagagtagactatatggatggtgtagtttctaaattgttttatgcctctaaattgtttaatgaaggttaacaatggtcttctattctattctgcagtcctaccctcataagattttaactgggaggagagacaaaatgccaactttaagacagacagatggactgtcagggttcactaagcgatctgaaagtgaatatgattgctttggtgctggtcacagttctaccaccatctctgctggcctaggtaatttgtttcttctggtcaagaattgagtttggaattggtaggatttttacattaactgaaaaggacctcaatgtttaagttatatatgaaaatcctttgggggggggggggtgttctggattcttttggcatagttgtttgtgctgtaaatatccatgaaaacctatcctacttcatctcactctagtagatgtccctttattgcgcaacatgacaatagctctttattgatattattaatct

SEQ ID NO: 27 - DNA, 6034 bp, Coffea canephora
  misc_feature (1533)..(1927): n is a, c, g, t or u
agattaaacccaccgggcattggctaatgaatgagtgagatcagatctcccatcttccttccttctttgattattggccctctttcgttttcgcttccttctcacttcacttctccccactgtcactgtccactccaccaaagccagctctctccctctctcacaaggctctttgcattgcatctgttctcctctacatctaaccgactaataccacaccaggagtgaccggtgaattcaaattttacatttccccaacctcagagcccacgttcataatcccattccccagaaagggtaaaaaaaaaaaaaggaaaggaaaagggaaaaaaaaaaccagtcttggcaaactttttcccacatttttacgccattttctctctcgcatgctcgcaaaattcttgtgaaaatgagtacttctttaaaatctgctaaaccctcaaaacccccaaacccatcctccgcccagacaaccccttcaagatcttcctcctcctcgctttcttcccatttagccatgattgaactcaagcaaagaatcctcacttctctctccaagctctccgacagagacacgcatcagatcgccgttgaagacctggagaaaatcgtccacaccctctccaacgatggcgtttcaatgcttctcaactgcctttacgacgcctccaacgaccctaaacccgccgtcaaaaaagaatccctccgccttctggcagtcctatgtgcttcccataccgattcggcttccacccacttgactaaaatcatagcccacattgtcaaaaggcttagagactcggattctgctgtcagggattcctgtcgggatgccattggatctttggcatccctttacttgaagggggaagctgcggctgatcatggtaatgtgggattgaattcagtagtctcgttgtttgtgaagccgctgtttgaatccatgagtgagaataataaggtggttcaaggtggggccgcaatgtgtatggctaaaatggtggaatctgcttccgacccgcctacaatggctttccagaagctgtgtcccaggatttgcaagtacctcaatagtcccaattttatggcaaaggcagcattgttgcctgttgtttccagcttatcccaggtttgttcattttaggcacatgttttctccaatatttttctagtggacaattctttgtttatggtgattaagctgtagtacatcttttatttcattagttttcttgtgtgttattgttagtgaggattcacgtctagtcagtattttcccggttatggagaattttctcttaagaagcgatcaaaaactctttatgaaattgaaatgttaatttttttagtcaggttggtattttaagtattttgcgagggtcatggagttgcatgctatatgtatttcatggttagaagaaaggcaatttgatattttgttaggcatagaaccatgatgtcccaatgcaatggcaagcaacgcctactatagctttatatatatatatatatatatatatatatatatatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatatatatatatatatatatatatatatatatatatatatatatatatatagggcagctggacacgaaaatgtagtctgcctctcttaccctgcttaatcagcattttcctttgccttcgttcggtcaagcattctttgcagtttattgtattgatgtgattttttttcatttcgagtagttaatgatttcagcctctgttttcattgagatttgctggtgcagaatttggaaatgcttgatactgtcattttatctgtgcaccaggaagaaatgtactgtttatatgcaatagctgccacacatgtggttctgtgttttgcatcttcgaaagtttttttaactctttatccttactgcccaatgtaccatatggatgcatcagaaaaaactaagacgtgatgcagtcttcacatatattggaaatataatggtgtaaacacaaaactgagattatgttctacttgtttcaggtgggagctgttgcacctcaaagccttgaatctttggtgcaaagtattcacgactgtcttagcagttcagattgggcgacacgtaaggctgcagctgagacattaattgttctggcattgcactctagcagcttggtggtagagggagctaactccacagtgactctgctagaggcttgccggtttgacaaagtagcctttttgtaatttttgtcgtttaaaaaatcatcctgtcgcaataaatatgctgtgtggttccgaatgggcagtctccttatgcaggagtttttttcttgaatactgataatggaaccaagaaaagtcaatagcctgataaatgcatcaatgctagctatcgtgcatttttccttttaggttttatttgcacaaggattttggaatattcttatgttgacattttgtttttgtttttttttttctgaagcttctaatatttctcattcatgaatactttatctattttagtcccatttagattgctagtctgttattttacataacacaaaagataatggggaccaagatgaacatcttaaaagaatgtatagcttttgagaaatttcttgtgagtatttgaaaatgtttctttagttgatgaacttcaaaatgtctttcatgctttcatcatactttcatttgattttgccaaggagtgcaggattagaattaatctaacctattgatgacatattccacgtcttactctcagttctcttgttctttttatggttatgtaggaggatacatgattggaataatatcactctttgggtttaatcagtatattaatgatgctaacatttgcagtccctttttcttccaacatggagaagagttgcttacttacaaattgtaaaaatgtttgtgcagactttgaattaaaattccaggacactcttgctttcaggaaaactacaaaaatgattaatattatttcttttcaatttttgatacagctggtctattcctcagcatttaacgggttttctatgtatttcaaatgtaatgaccatatgtaatgttgtagttgttgattttctctttcaatttgacagagatctcaaccatctgaatctttttctacttcccttttttttttcagttctcctttttaatgagataagtgaagttattaagtattgcaagtatctcgacatacaaaagctaaaagttaacatgttcactctctgtcccagataaaaccagtcagagatagtgtaactgaagctctgcagctgtggaagaaaattgcaggaaaaggagatggagcttcagatgaacacaaaccttcatctcacagtatgcttgacttcaatatattaaatttcagtttctccttcaattaggatttcctgttaattctcttaagcccagtgttttcttatgtctcttcagatggtgagacttctgaatcagcttatccatcagacaaggactctcgaaaccctggtgaaagaagtgaactaccggtgaaggatttatctaataatccatcttctaatgatgcatatctcaaagacaagggtagcaacattatggacaaggcagttgggatactgaggaagaaggcacctgcattaactgacaaagaattgaaccctgagtttttccaaaaacttgaaacaaggggttcagatgatttgcctgtagaagtggttgtccctcgtcgatgccctaattcttctaatttgcagaatgaggaagaggctgtgggcaaggattcaagggagaggacaaggaccagctaccagcctgatggtggatcacttgactttagatatcgtaacactgagaaaggaacttctagctatagttctagagaacgagatactgatgaaacaagtgatctgaatcaaagagatttatctggcattcaagggggtttttccaagagtggaggccaatctgacagtttctcgaataataaaggaaattggctggctattcagaggcaattattacaactggaaaggcagcaggctcatctcatgaacatgttgcaggtgaggttcaataacatataactgcaaggaattattcctgtatctccagttgtgctaaccttttcctcatattgtaggattttatgggtggttcacatgatagcatggtgacgcttgaaaacagagtgagaggtcttgaaagagtagttgaagacatggcacgggatttgtctctatcaacaagtcgaagaggtgctagttttatgggtggatttgaaggatcatccaacagaagtgcagggaaatacaatgcctttgctgactatactaatgctaaattagggagtggtagtgatggaaggattccctttggagatagatttgcaccttctgatggtagaccttcaggcaataggggaaggggccctccttggagatctgatgcacctgatgcttgggattttcaagcatatggtaaaaatgggcaaatgggttctagaagaactttgggtggtggtcctgttgattgtaggtcccctaaatccgaaaatgataatgatcaagttggcagcaggagagcttgggaccgaggagctggacctgttagatttggtgagggaccatctgctagaagtgtctggcaagcttcaaaggatgaggcaacattagaagcaataagggtagctggtgaagacagtggggctgctcgaagtgcaagggtagcagtgccagaattgactgctgaagcattaggggatgataatgtcatgcaagaaagagatcctatctggaattcttggagcaatgctatggatgcacttcatgttggtgatacagattcagcttttgctgaagttctatctagtggagatgatcttctgcttgtaaagttaatggacagatcagggcctgtattagatcaaatctcaagtgaggttgcaattgaggttttacatgccattgcccaatttttactcgagcaggacttgtatgacatcagcttatcctgggtgcaacaggtattgtcactcttgattattgcctgactttctttcactgcattgagatctatattattgtcaaaatggtttcatgaatccaggttcctttcacagttcttgtctcaatttctcaagttgaaagtcatgaattatgtgattaaaatgatgaaggcatacaaagccatgagttctagcctcttttgcaatttatgtctgtcacttctactgtcaaatggtataggataaattctcagtagtatgtttttataataaaaggacgacttctaattaactggaagcagtcgagtaattttgtctaaaaagtggggcggttttttgttaaatggtctacaagcaaccttataggactttgttgcacggaggctgcagggttttctgatcttcttattatgttttttcattctggcttccttagtttcacactaaatgatctcatttctcatctcgtcagttggtggaaattacggtagaaaacgggactgacgttcttggcattcctatggatgtgaaaagagaaattttgttgaatttacatgaagcttcttcagcaattgatgtgccagaggactgggaaggagcaacaccagaacaacttttgttccagttggcatctgcttgggaaattgacttgaagcaattggagaaatag

SEQ ID NO: 28 - DNA, 23 bp, Artificial sequence
  sgRNA sequence
tttctgcactaagcctgaccagg

SEQ ID NO: 29 - DNA, 23 bp, Artificial sequence
  sgRNA sequence
tgtcgtgagatcaagggcggagg

SEQ ID NO: 30 - DNA, 23 bp, Artificial sequence
  sgRNA sequence
tcgtctcagattccataaagcgg

SEQ ID NO: 31 - DNA, 23 bp, Artificial sequence
  sgRNA sequence
tctattctagctggctctatggg

SEQ ID NO: 32 - DNA, 23 bp, Artificial sequence
  sgRNA sequence
agagcttggagagagaagtgagg

SEQ ID NO: 33 - DNA, 23 bp, Artificial sequence
  sgRNA sequence
gtccacaccctctccaacgatgg

SEQ ID NO: 34 - DNA, 20 bp, Artificial sequence
  sgRNA sequence
gggcgaggagctgttcaccg

SEQ ID NO: 35 - DNA, 20 bp, Artificial sequence
  sgRNA sequence
ggccacaagttcagcgtgtc

SEQ ID NO: 36 - protein, 9 aa, Artificial sequence
  LAGLIDADG motif amino acid sequence
LAGLIDADG

SEQ ID NO: 37 - protein, 241 aa, Artificial sequence
  EGFP amino acid sequence
MSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - protein, 591 aa, Musa acuminata
MKPRVVAHSKARSGGKAAVPQQAVFEMKQRVILLLNKLADRDTYNIGVEELEKAALRLTPDMIAPFLSCVTETNAEQKSAVRAECVRLMGTLARSHRILLAPYLGKVVGSIVKRLKDTDSVVRDACVEACGVLATSIRGGEGGGGATFVALAKPLFEALGEQNRYVQVGAAHCLARVIDEASDAPQNILPQMLTRVIKLLKNQHFMAKPAIIELIRSIIQAGCALAEHTLSAAVTSILEALKSNDWTTRKAASVALAGIAVNPGSSLAPLRSSCLHFLESCRFDKVKPARDSIMHAIQCWRALPVTHSSETSEAGSSTKGITVSGKMIEECLDTLSRKNGPVSDLCGNSTSSTQKRAPLSVRKPCTTNMQSHQRMKSNDWHIAMSVPKTHGTPLVNSNSVKSDSNVIDLLERRMLNTAELQNINFDYGSVFDKTECSSVSVPDYRIYEMEHLTVSHDCDGENDSEGNDSISPTRNNHSAIEDNGRECLGTQERKSPESTISDLCSRSMHGCCVHAANGLAAIKQQLLEIETKQSNLLDLLQIIENCILFHSPNYNKKFSDSIRFSTTNDIWFNFNFYIRLVKISYLAQFVD

SEQ ID NO: 39 - protein, 749 aa, Musa acuminata
MATSTSKPSSRLSKPSSSSSKSQSCSSSSSGLSSHVAMVELKSRILAALAKLSDRDTHQIAVDDLEKIIRTLPAEGVPVLLNALDSAGGIGGSSSMVALFVKPLFDAMGEQNKAVQGGAAMCLARVVECAGANDDGGEGEEGRVTASGTMLQRLCPRICKLLGGQSFLAKGALLSVVSSLAQVGAIHLQSMQQLLQIVRECLESSEWATRKAAADTLCVLASHSSHLLGDGAAATITALDACRFDKVKPVRDSMMEALQLWKKIKGQGEDSRNSDLTDSEEKATHKRSNSNKRSETLKNSSAGSSPSENDSVSRGKGTNMPEKAVILLKKKAPSLTDKELNPDFFQKLEKKSSDDLPVEVVLPRNCLQSSHSQCEEGPEAIYSDSTETPKHNSDYFPRGRWIEQRGIRAKESKAEDFDGSFMSNKANWSAIQRQLAQLERQQISLMNMLQDFMGGSHDSMVTLENRVRGLERVVDEMARDLAIKPGRRVRRRGSPWRSESETWDYHGASRNGVVNSRRGFNAVPVDGRVPRSEHDADQVGGRWAWDKGPGPFRLGEGPSARSVWQASKDEATLEAIRVAGEDNITSITAARVAVPELDAEGIADDNLGLDKGPLWASWTRAMDSLYVGDVDSAYAEILSTGDDLLLVKLMDKSGPVFDQLSNEIASEVFRAIGQFVLEESLFDIALSWLHQLSDLVVENGSEFLSIPLEWKREMLLNLREASVSEPPEYWEGTPPDQLMMHLAAAWGLN

SEQ ID NO: 40 - protein, 861 aa, Musa acuminata
MVELKSRVLSALSKLSDRDTHQIAVDDLEKIIRTLPADGVPMLLHALIHDPSMPSPSPQDPPGSKNPSFLVGRRESLRLLALLCASHTDAASAHLPRIMAHIVRRLKDPASDSSVRDACRDAAGSLAALYLRPSLAAAAAHVDGAGSGGPSPVVALFVKPLFEAMGEQNKAVQGGAAMCLAKVVESAGGGGVGGGGQREEGRVMTTGVVFQKLCPRICKLLGGQSFLAKGALLSVISSLAQVGAISPQSMQQVLQTIRECLENSDWATRKAAADTLCVLASHSSHVLGDGATATITALEACRFDKVKPVRDSMMEALQLWKKIRGDGTLADTKGISDLTDNEEKEDHKSDSVSKENGTNMLEKATVLLMKKAPSLTDKELNPEFFQKLEKRSLDDFPVEVVLPRRCLQSSHSQCEEGSEKVQDLDNFARDKWTEQRGSKAKESKAKVLNVEDTTEVCQKDPSPGRTNVPRSDANTDGPFMSNRANWTAIQRQLAQLERQQASLMNMLQDFIGGSHDSMVTLENRVRGLERVVEEMAHDLAMSSGRRVGNMMLGFDKSPGRSSSKYNGLHDYSSSKFGRVGERFHLSDGLVTGVRGRDSPWRSESEAWDSYGYVASRNGVMNTRRGFGAVPVDGRLHKTEHDTDQVSGRRAWNKGPGPFRLGEGPSARSVWQASKDEATLEAIRVAGEDNGTSRNAARVAVPELDAEALTDDNPGPDKGPLWASWTRAMDSLHVGDIDSAYEEILSTGDDLLLVKLMDKSGPVFDQLSGEIASEVLHAVGQFILEQSLFDIALNWLQQLSDLVVENGADFLRVPLEWKREILLNLHEASALELPEDWEGAAPDQLMMHLASAWGLNLQQLVK

SEQ ID NO: 41 - protein, 635 aa, Musa acuminata
MKNLSVRELKQLADELRSDIIFNVSRTGGHLGSSLGVVELTVALHYVFNAPQDKILWDVGHQSYPHKILTGRRDKMATMRQTNGLSGFTKRSESEYDCFGAGHSSTSISAALGMAVGRDLKGRKNNVVAVIGDGAMTAGQAYEAMNNAGYLDSDMIVILNDNKQVSLPTATLDGPVPPVGALSSALSRLQSSKPLRELREVAKGVTKQIGGSMHEIAAKVDEYARGMIGGSGSTLFEELGLYYIGPVDGHNIDDLVAILKDVKSTKTTGPVLIHVVTEKGRGYPYAEKAADKYHGVAKFDPATGKQFKSGSKTQSYTNYFAEALIAEAEVDEGIVAIHAAMGGGTGLNYFLRCYPTRCFDVGIAEQHAVTFAAGLACEGLKPFCAIYSSFLQRAYDQVIHDVDLQKLPVRFAMDRAGLVGADGPTHCGSFDVTYMACLPNMVVMAPSDEAELFHMVATAAAIDDRPSCFRYPRGNGIGVPLPPGNKGIPLEVGKGRILKEGERVTLLGYGTAVQSCLAAASLLEERGLKITVADARFCKPLDRSLIRNLARSHEVLLTVEEGSIGGFGSHVVQFLALDGLLDGTLKWRPVVLPDRYIDHGSPRDQLAEAGLTPSHIAATVLNILGQTREALEIMS

SEQ ID NO: 42 - protein, 748 aa, Musa acuminata
MAASTLPFSCHLPALLSSDLQKASPLLPTQLFAGTDLPHHRHRHGFLTPRRRSCVCASLSGTGEYFSQRPPTPLLDTVNYPIHMKNLSVKELKQLADELRSDVIFHVSKTGGHLGSSLGVVELTVALHYVFNAPQDKILWDVGHQSYPHKILTGRRDKMPTLRRTNGLSGFTKRSESDYDSFGTGHSSTSISAALGMAVGRDLKGRKNNVIAVIGDGAMTAGQAYEAMNNAGYLDSDMIVILNDNKQVSLPTASLDGPIPPVGALSSALSRLQSSRPLRELREVAKGVTKQIGGSMHQIAAKVDEYARGMISGSGSTLFEELGLYYIGPVDGHNIDDLVSILKEVKDTKTTGPVLIHVVTEKGRGYPYAERAADKYHGVTKFDPATGKQLKSISQTQSYTNYFAEALIAEAEVDKDIVAIHAAMGGGTGLNYFLRRFPTRCFDVGIAEQHAVTFAAGLACEGLKPFCAIYSSFLQRAYDQASHCPHFSILSFDKVKPTRSSNDEFELLMQVIHDVDLQKLPVRFAMDRAGLVGADGPTHCGAFDVTYMACLPNMIVMAPSDEAELFHMVATAAAINDRPSCFRYPRGNGIGVPLPQGNKGVPLEIGKGRILIEGERVALLGYGTAVQSCVAAASLLEQRGLRVTVADARFCKPLDHALIRNLSKSHQVLITVEEGSIGGFGSHVAQFMALNGLLDGTIKWRPLVLPDRYIEHGSPNDQLAEAGLTPSHVAATVLNILGQTREALEIMS

SEQ ID NO: 43 - protein, 408 aa, Musa acuminata
MISTDGSLLFEELGLYYIGPVDGHNVEDLVTIFEKVKSLPAPGPVLIHIVTEKGKGYPPAEAAADKMHGVVKFDPRTGKQFKSTSSTLSYTQYFAESLIKEAEADDKIVAIHAAMGSGTGLNLFQHKFPQRCFDVGIAEQHAVTFAAGLATEGLKPFCAIYSSFLQRGYDQVVHDVDLQKIPVRFALDRAGLVGADGPTHCGAFDITYMACLPNMIVMAPADEAELVHMVATAAAIDDRPSCFRFPRGNGVGVMLPPGNKGTPFEIGKGRVLMEGNRVAILGYGSIVQTCLKAADPLRARGVFATVADARFCKPLDVGLIRRLVNEHEILITVEEGSIGGFASHVTHFLSLSGLLDGRMKLRPMVLPDRYIDHGSPQDQIEAAGLSSGHIVSTVLNLLGRQKEALYLH

SEQ ID NO: 44 - protein, 710 aa, Musa acuminata
MASASSHCPFRHISFLQSESRFQSAESGYFGTPQFLKKSTSELIIYQNSVTTYLRKGCRQVAALPDIGDFFWEKDPTPILDMVDMPIQLKNLSHKELKQLAGEIRSEISFVMLKTRRPFRASLAVVELTVALHHVFHAPMDKILWDDGEQTYAHKILTGRRSLMHTLKRKDGLSGFTSRAESEYDAFGAGHGCNSISAGLGMAVARDINGKKNRIVTVISNWTTMAGQVYEAMSNAGYLDSNMIVILNDSRHSLHPKLSEGPKMTINPISSTLSKIQSSRSFRRFREAAKGVTKRIGKTMHELAAKVDEYTRGMIGPLGATLFEELGLYYIGPVDGHNIDDLICVLNEVASLDSTGPVLVHVITEDEDLESIQKENSKSCSNSINSNPSRTFNDCLAEAIVAEAERDKEIVVVHAGMGVDPSLKLFQSRFPDRFFDVGMAEQHAITFAAGLSCGGLKPFCIIPSTFLQRGYDQVIQDVDLQRLPVRFAISSAGLAGSEGPIHSGVFDITFMACLPNMIVMAPSDEDELIDMVATAACVNDRPICFRYPRVAIMGNNGLLHSGMPLEIGKGEMLVEGKHVALLGYGVMVQNCLKAQSLLAGLGIQVTVASARFCKPLDIELIRRLCQEHEFLITVEEGTVGGFGSHVSQFMALDGLLDGRVKWRPILLPDNYIEQATPREQLEIAGLTGHHIAATTLSLLGRHREAFLLMR

SEQ ID NO: 45 - protein, 691 aa, Musa acuminata
MVEARSLMVASAAPFLKALSSSANGRRQLCVRAGGASGDGKVMITKEKSGWKIDYSGEKPATPLLDSINYPIHMKNLSTRDLEQLSAELRAEIVFAVAKTGGHLSSSLGVVELAVALHHVFDAPEDKIIWDVGHQAYPHKILTGRRSRMNTIRQTAGLAGFPKRDESIYDAFGAGHSSTSISAGLGMAVARDLLGKKNHVISVIGDGAMTAGQAYEAMNNAGYLDSNLIIVLNDNKQVSLPTATLDGPATPVGALSKALTKLQSSTKLRKLREAAKNITKQIGGQTHDIAAKVDEYARGMMSATGYSLFEELGLYYIGPVDGHDVEDLVTIFEKVKSLPAPGPVLIHIVTEKGKGYPPAESAADKMHGVVKFDPKTGKQFKSKSSTLSYTQYFAETLIKEAQVDDKIVAVHAAMGSGTGLNYFQHKFPERCFDVGIAEQHAVTFAAGLATEGLKPFCAIYSSFLQRGYDQVVHDVDLQKIPVRFALDRAGLVGADGPTHCGAFDIVYMACLPNMIVMAPADEAELMHMIATAAAIDDRPSCFRFPRGNGVGVALPPNNKGTPLEIGKGRVLMEGNRVAILGYGSIVQTCLKAADSLRSHGIFPTVADARFCKPLDVELIRRLANEHEILITVEEGSIGGFGSHLRSMVLPDRYIDHGSPQDQFEVAGLSSRHIAATVLSLLGRRKEALHLH

SEQ ID NO: 46 - protein, 707 aa, Musa acuminata
MEASGSLMAAFSAPFLVAPNPRTSPKRQFRVRACGLGGDGKMMFNKGKSGWTIDFSGEKPPTPLLDTINYPIHMKNLSVQDLEQLAAELRAEIVFTVSKTGGHLSASLGVVELSVALHHVFDTPEDKIIWDVGHQAYTHKILTGRRSRMHTVRQTSGIAGFPRRDESIYDAFGAGHSSTSISAGLGMAVARDMLGKKNHVISVIGDGAMTAGQAYEAMNNSGYLNSNLIVVLNDNRQVSLPTATLDGPATPVGALSKALTRLQASTKFRKLREAAKSITKQIGGPTHEVAAKVDEFARGLISANGSSLFEELGLYYIGPVDGHNLEDLVTIFQDVKSMPAPGPVLIHIVTEKGKGYPPAEAAPDKMHGVVKFDPSTGKQLKPKSPTRSYTQYFAEALIKEAEADNKVVAIHAAMGGGTGLNYFQKRFPDRCFDVGIAEQHAVTFAAGLATEGLKPFCAIYSSFLQRGYDQVVHDVDLQKIPVRFALDRAGLVGADGPTHCGAFDITYMACLPNMIVMAPADEAELMHMVATAAAIDDRPSCFRFPRGNGVGVALPPDNKGSPLEIGKGRVLMEGDRAAILGYGSTVNTCLKAADTLRAHAVFATVADARFCKPLDVKLIRSLVKEHDILITVEEGSIGGFGSHVAHFLSLSGLLDGQLKLRSMVLPDRYIDHGSPQDQIEAAGLSSRHVAATVLSLLGRRKEALLLK

SEQ ID NO: 47 - DNA, 2898 bp, Musa acuminata; identical to SEQ ID NO: 9

SEQ ID NO: 48 - DNA, 2913 bp, Musa acuminata; identical to SEQ ID NO: 8

SEQ ID NO: 49 - DNA, 1776 bp, Musa acuminata
atgaagccccgcgtcgtggcgcattccaaggccagatcgggcggaaaggcggccgtgccgcagcaggccgtcttcgagatgaagcaacgggtgatcctcttgctgaacaagctcgccgaccgcgacacgtacaatatcggcgtggaagagctcgagaaggccgctttgaggttgacccccgacatgatcgctcctttcctgtcgtgcgtcaccgagaccaatgccgagcagaagagcgccgtccgcgcggagtgtgtccgactgatgggtaccctggcgaggtcccataggatcctcttggctccctatctcggcaaggtggtcggttccatcgtcaagcgcctcaaggacacggattccgtcgtccgtgacgcctgcgtcgaggcgtgcggcgttttggcgaccagcattagaggcggggaaggcggcggaggggcaacgttcgttgcattggccaagccccttttcgaagctttgggtgagcagaaccgatacgtgcaggtgggtgcggcgcactgcttagcgagggtcatcgatgaggccagtgatgctccgcagaacatcttgccacagatgctcacgcgtgtcataaagctgctgaagaatcagcatttcatggctaagccggcgatcattgagttgatcagaagcatcatacaggcaggatgtgctttagcagagcatactttatctgctgcagttacgagcattttggaagctcttaaaagtaatgattggacaacacgcaaagctgcttctgtggcattggctggaatcgccgtcaaccctggatcttctttggctcctctgagaagttcttgcctccacttccttgaatcctgcagatttgacaaagtgaaacctgcgcgggattcaatcatgcatgccatacagtgttggagagctctcccagtgacccattcttctgaaacttcagaggctggatcatccacaaaaggtataactgtttctgggaaaatgatcgaagaatgcttagacacattgtctagaaaaaatggtcctgtttctgacttatgtggaaattccaccagttcaacacaaaaaagagctcctctatctgtcaggaaaccatgtacaactaatatgcagagtcatcaacgtatgaagtcaaacgattggcacattgcgatgtcagtccccaagactcatggtacaccattggttaatagcaatagtgtaaagtctgacagtaatgtaatagatcttttagaaagaaggatgctaaatactgctgaactccaaaatatcaactttgattatggttctgtgtttgataagacagaatgctcttccgtatccgttccagattatcggatctatgagatggagcatttaactgtatctcatgactgtgatggggagaatgattctgagggcaatgattcaataagtccaacaagaaataatcattctgccattgaggacaatggacgagaatgccttggtacccaggagcggaagagtccggagtccactatttcagatttgtgttcacgcagtatgcatggatgttgtgtgcatgctgcaaatggactggctgccatcaaacagcaactcctagaaattgaaacaaaacaatcaaatttgctggatctcttacagattatagaaaattgtatccttttccactctccaaactataacaaaaaattttctgatagcatccgtttttccacaactaatgatatttggtttaattttaatttttacataagattggtcaaaatttcatatctagcccagtttgtggactaa

SEQ ID NO: 50 - DNA, 2250 bp, Musa acuminata
atggctacttccacctccaaaccctcttctaggctctccaaaccctcttcctcctcttccaaatcccaatcttgctcttcctcctcttctggcctttcctcccatgtcgccatggtggagctcaagtcgcggatcctcgcggcgctcgcgaagctatccgatcgcgacacccaccagatcgccgtcgacgacctcgagaagatcatccgcaccctccccgccgagggcgtccccgtgctcctcaacgccctcgatagcgctggcggcatcggagggtcatcttcgatggtggcgctgttcgtgaagccgctgttcgacgccatgggggagcagaataaggcggtgcaaggcggggcagccatgtgccttgctagggtggtggagtgtgccggggctaacgatgatggtggggagggggaggagggaagggtgacggcgtcggggacgatgctccagaggttgtgccccaggatctgtaaacttcttggaggccagagctttcttgccaagggggcgttgctttcagttgtctctagcttggcgcaggtaggagcgatacatctgcagagcatgcaacaactgctgcaaattgttcgtgaatgtcttgaaagcagtgaatgggctacccgtaaggcagctgcagacacattgtgtgtcttggcctctcactcgagtcatttgcttggtgatggagctgcagcaacaataactgctcttgacgcttgccgttttgataaggtaaaacctgtcagagatagcatgatggaggcactgcagctatggaagaagatcaaaggacaaggagaggattctagaaactctgacttaactgatagtgaggaaaaggcaactcacaagaggtccaactctaataagaggtcagaaactttgaaaaactcatctgctggttcttcacccagtgaaaatgattctgtatccagaggaaaaggcactaatatgcctgagaaagcagtcatactgttaaagaaaaaagcaccatctttgactgacaaagaattgaacccagacttcttccaaaagcttgagaagaagagttcagatgacctgccagtagaagtagtgttacctcgtaactgtttgcagtcttcccattcacaatgtgaagaaggaccagaagcaatttatagtgattcaacggaaacaccaaagcataactcggattattttcctagggggagatggatagagcaaagaggtatcagagcaaaagaatcaaaagcagaggattttgatgggtcctttatgagcaataaagcgaattggtctgccatacagaggcagctagcccaattagagaggcaacaaatcagtcttatgaacatgttacaggactttatgggaggttcccatgatagcatggtaactctagaaaatcgagtgaggggtcttgagagagttgttgatgaaatggcccgtgatttggctattaaaccaggaaggagagtaaggagacgaggttctccttggaggtcagaatctgaaacatgggattaccatggtgcctcaaggaatggtgtcgtgaactctaggagagggttcaatgctgttccagtggatggtagagtacctagatctgagcatgacgctgatcaagttggtggcaggtgggcctgggataagggaccaggaccatttaggcttggtgaagggccttctgcaagaagtgtttggcaagcctcaaaggatgaggctactttagaagctatccgagtagctggggaagacaacataacatccataactgcagcacgagtagctgttcctgaattagatgctgaaggtatagcagatgataatctggggctggacaagggtccactttgggcttcgtggactcgtgcgatggattcactttatgttggcgatgttgattcagcttatgcagagattctgtctactggtgatgacttattacttgtaaagctaatggataaatctggtccagtatttgatcagctctctaatgaaatagcgagcgaagtctttcgtgcaattggacagtttgttctggaagaaagcttgtttgatatagcgcttagctggctccatcagttatcggatcttgtcgtggagaatggaagcgagtttctcagcatccccctcgaatggaagagagagatgttgctgaatcttcgtgaagcttctgtttcagaaccaccagaatattgggaggggacaccaccggatcagctaatgatgcatttagcggctgcatggggtctcaactag

SEQ ID NO: 51 - DNA, 2586 bp, Musa acuminata
atggtggaactcaaatcgcgggtcctgtcggcgctgtcgaagctttccgaccgcgacacccaccagatcgcggtcgacgacctggagaagatcatccggaccctccccgccgacggcgtccccatgctcctccacgccctcatccacgacccctccatgccctcgcccagcccccaggacccgcccgggtccaagaacccctccttcctcgtgggtcgccgcgagtccctccgcctcctcgcgctcctctgcgcctcccacaccgacgccgcttccgcgcacctccccaggatcatggcccacatcgtccgccgcctcaaggaccccgcctccgactcctccgttcgcgacgcctgccgtgacgccgccggttcgctcgccgcgctctatctccgcccctcgctcgcagcggcggccgctcatgtggacggcgctggcagcggaggaccgtctccggtggtggcgttgttcgtgaagccattgtttgaggccatgggggagcagaataaggcggtgcagggcggggctgccatgtgcctcgcgaaggtggtcgagtctgctggaggtggcggcgtcggcggtggtgggcaaagggaggagggaagggtgatgacgacaggagtggttttccagaagttgtgccctaggatctgtaagctgcttggtggccagagctttctagctaaaggagcattgctttcagtcatctctagccttgctcaggtaggagcaatcagtcctcagagcatgcaacaagtgctgcaaactattcgtgaatgtcttgagaatagtgactgggctacccgtaaggcagctgctgatacactctgtgtgttggcctctcactcgagccatgttcttggtgatggggctacagcaaccataactgctcttgaggcctgccgttttgataaggtaaaacctgttagagatagcatgatggaggcactgcagctatggaagaagattagaggagatggaactttggcagacacaaaaggcatctcggacttaactgataatgaagaaaaggaagatcataaaagtgactctgtctccaaagaaaatggcaccaacatgctagagaaagcaacagtgcttttaatgaaaaaagcaccatcattaaccgataaggagttgaatccagaattcttccaaaagctagagaagaggagtttggatgactttcctgttgaagtggtgctacctcgtaggtgcttacagtcttcccattctcaatgtgaagaaggatcagaaaaggtgcaggacttggataattttgctcgggacaaatggacagagcaaaggggatctaaggcaaaagaatcaaaagcaaaagttttgaatgttgaggacacaactgaagtctgtcagaaagatccttctcctggtcgtacaaatgtccctagatctgatgccaacactgatgggccttttatgagcaatagggcgaattggactgcgatacagaggcagttggctcaattagagaggcaacaagccagtctcatgaatatgttacaggacttcattggtggctcccatgatagtatggtaactctagaaaatagagttaggggtcttgagagagttgttgaagaaatggctcatgatttggctatgtcatctggaaggagagttggaaatatgatgctgggatttgacaaatctccaggaaggtcttcaagcaagtacaatggccttcatgattactccagctcaaagtttggcagagttggtgaaaggtttcacttgtcagacggtttggtaactggtgttcggggaagagattctccgtggaggtcggaatctgaagcatgggattcctatggatatgtagcttcaagaaatggtgttatgaacactaggagagggtttggtgctgttccggtggatggtaggttacacaaaaccgagcatgatactgatcaagtcagtggtaggcgggcttggaacaaaggaccaggaccgtttaggcttggtgaagggccttctgcaagaagcgtttggcaagcctcaaaggatgaggctacacttgaagctatcagagtagctggggaagacaatggaacatccagaaatgcagcacgagtagctgtaccagaattagatgctgaagctttaacagatgataatccagggcccgacaagggtccactttgggcgtcttggactcgtgccatggattcacttcatgttggtgacattgattcagcttatgaagagattctatctactggtgatgacttattacttgtaaagctaatggataaatcaggtccagttttcgaccagctctctggtgaaatagcaagtgaagtcttgcacgcagttgggcaatttattctggagcaaagcttgtttgatatagcattgaattggcttcaacagttgtcagatcttgttgtagagaatggagccgacttccttagagtccccctcgaatggaagagagagattttgttaaatcttcatgaagcttctgcacttgaactaccagaggattgggagggggcagcaccagaccaattaatgatgcatttagcatcagcctggggtctcaacttgcaacagcttgtcaagtag

SEQ ID NO: 52 - DNA, 2157 bp, Musa acuminata; identical to SEQ ID NO: 7

SEQ ID NO: 53 - DNA, 1941 bp, Musa acuminata
atgcgttcaattcccctgaggacaagatcatttgggatgtcggccatcaggttagattcctctcttggtagtatccaaagcaattacttttctgttttgctttgcaaattgggtaacgaagaaggattgagtgaagaacaggcctatcctcataaaatattgactggaagaaggtcaagaatgcacaccatcagacaaacctcagggcttgcgggattccccaagagagatgagagcatccatgatgcctttggtgctggtcatagttccacgagcatctctgcggggcttggaatggctgtcgcaagagatctgctagggaagaagaatcatgttgtgtccgtgatcggtgatggagccatgactgctgggcaggcgtatgaggccatgaacaatgctggctacttggactctaaccttgttatcgtgttgaatgataacaagcaagtttccttgccgactgcaacccttgacggaccagccactcctgttggggcactcagtaaggccctcaccaaacttcagtccagcacagagttccgtatgcttcgtgaagcagctaagaatctcacaaagcagattggtgagcgaacacacgagattgctgcaaaagtggatcaatatgctcgaggaatgataagcactgatgggtctttgttattcgaagagctcggtctctattatattggacctgtagatgggcacaatgtagaagacttggttaccatctttgagaaggtgaagtctttgcctgctccaggacctgtccttatccatattgtgacagagaaaggaaaggggtatccccctgctgaggcggctgctgacaaaatgcatggtgagcattatttgctgcttgtaatgcatgtgcccgacttcttcccgactgtcataatgatccatgtgtttcttgctgtaggtgttgtgaagttcgacccaagaactgggaaacaattcaagtcaacatcatcgaccctttcatacactcagtactttgccgaatctctcattaaagaagcagaggccgacgacaagattgtggccattcatgctgccatgggaagtgggacggggctgaacttgtttcaacacaagtttcctcaaagatgctttgatgtggggattgcagagcagcatgcagtcacctttgcagccggtctggccaccgaaggcctcaagcctttctgtgccatctattcctcgtttctgcaacgaggatatgatcaggtggttcatgatgtggatttacagaagatacctgtccgtttcgctctggatcgagctggtcttgtcggagctgatggacctacacactgtggagcatttgacatcacgtacatggcatgtttgcccaacatgattgtaatggctccagctgatgaagctgagctagtgcacatggtcgcaacagcagcagcaatcgacgacagacctagctgcttcagattcccaaggggcaatggagttggtgtgatgcttcctccgggcaacaaaggcaccccttttgagattgggaagggaagggttctgatggaaggaaacagggtggccattcttggatatggttcaatagtacagacatgcttgaaggctgcagacccactgagagcccgtggagtttttgccaccgtagctgatgctcgtttctgtaagcctctggatgtggggctcataagaaggctggtaaatgagcatgagatcttgatcacagtggaggaaggctccattggaggtttcgcatcgcatgtcactcacttcttgagcttgagtggcctcctggatggccgcatgaagctgaggccaatggttctaccagaccgatacatcgaccatggatcacctcaggatcagattgaagcagctggactttcttcaggacatattgtaagcacagtgctgaatctgttaggcaggcagaaggaagcattatacctccattga

SEQ ID NO: 54 - DNA, 1905 bp, Musa acuminata
atgaagaatctctccacggaggatttagagcagttggcagcagagctgagagcagagattgtgttctcggtgtcccaaactggtggccacttgagtgcgagcttaggagtggtggagttggctgtggctctccatcatgcgttcaattcccctgaggacaagatcatttgggatgtcggccatcaggcctatcctcataaaatattgactggaagaaggtcaagaatgcacaccatcagacaaacctcagggcttgcgggattccccaagagagatgagagcatccatgatgcctttggtgctggtcatagttccacgagcatctctgcggggcttggaatggctgtcgcaagagatctgctagggaagaagaatcatgttgtgtccgtgatcggtgatggagccatgactgctgggcaggcgtatgaggccatgaacaatgctggctacttggactctaaccttgttatcgtgttgaatgataacaagcaagtttccttgccgactgcaacccttgacggaccagccactcctgttggggcactcagtaaggccctcaccaaacttcagtccagcacagagttccgtatgcttcgtgaagcagctaagaatctcacaaagcagattggtgagcgaacacacgagattgctgcaaaagtggatcaatatgctcgaggaatgataagcactgatgggtctttgttattcgaagagctcggtctctattatattggacctgtagatgggcacaatgtagaagacttggttaccatctttgagaaggtgaagtctttgcctgctccaggacctgtccttatccatattgtgacagagaaaggaaaggggtatccccctgctgaggcggctgctgacaaaatgcatggtgttgtgaagttcgacccaagaactgggaaacaattcaagtcaacatcatcgaccctttcatacactcagtactttgccgaatctctcattaaagaagcagaggccgacgacaagattgtggccattcatgctgccatgggaagtgggacggggctgaacttgtttcaacacaagtttcctcaaagatgctttgatgtggggattgcagagcagcatgcagtcacctttgcagccggtctggccaccgaaggcctcaagcctttctgtgccatctattcctcgtttctgcaacgaggatatgatcaggtggttcatgatgtggatttacagaagatacctgtccgtttcgctctggatcgagctggtcttgtcggagctgatggacctacacactgtggagcatttgacatcacgtacatggcatgtttgcccaacatgattgtaatggctccagctgatgaagctgagctagtgcacatggtcgcaacagcagcagcaatcgacgacagacctagctgcttcagattcccaaggggcaatggagttggtgtgatgcttcctccgggcaacaaaggcaccccttttgagattgggaagggaagggttctgatggaaggaaacagggtggccattcttggatatggttcaatagtacagacatgcttgaaggctgcagacccactgagagcccgtggagtttttgccaccgtagctgatgctcgtttctgtaagcctctggatgtggggctcataagaaggctggtaaatgagcatgagatcttgatcacagtggaggaaggctccattggaggtttcgcatcgcatgtcactcacttcttgagcttgagtggcctcctggatggccgcatgaagctgaggccaatggttctaccagaccgatacatcgaccatggatcacctcaggatcagattgaagcagctggactttcttcaggacatattgtaagcacagtgctgaatctgttaggcaggcagaaggaagcattatacctccattga

SEQ ID NO: 55 - DNA, 2133 bp, Musa acuminata
atggcctctgcttcctctcattgcccgttcagacatatttctttccttcaaagcgaatctaggttccaatctgcggaatctggttactttgggactccgcagttcttgaagaagagcacttctgagttgattatttaccaaaattctgtaactacgtatctaaggaagggttgcagacaggttgctgcactaccagatattggtgatttcttctgggaaaaagatccaactcccattttagacatggttgatatgccgattcaattgaagaatctgtcccacaaagaactaaagcaattagctggtgaaattcgttctgagatatcttttgttatgttaaagacccgtaggcccttcagagcaagtcttgcagtggtggagttaacagtggctttacatcatgtttttcatgctcccatggacaagatactctgggatgatggtgaacagacatatgcacacaagattctgacaggaaggcgctctcttatgcatacacttaagcgaaaagatggtctctcgggtttcacttctcgagcagaaagcgagtacgacgcatttggtgctgggcatggatgcaatagcatatctgctgggcttggcatggcagttgcaagggatattaatggaaagaagaatcgtatagtgacagttataagtaattggacaacgatggctggtcaggtctatgaggcaatgagcaatgctgggtatcttgattctaacatgatagtgattttaaatgatagtaggcactctttacaccctaagcttagtgaaggaccaaaaatgacaatcaatccgatctcaagcactttaagcaagattcaatctagtagatccttccggagattcagggaagctgcaaagggtgtaacgaaaagaatcggtaaaactatgcacgaattggcagctaaagtcgatgagtatacacgtggtatgattggtcctcttggagctactctctttgaagaacttgggctgtactacattggaccagtggatggacacaatattgatgatctaatttgtgtactcaatgaagtggcatcattggattcaactggacccgtattggttcatgtcattacagaagatgaggacttggaaagtattcagaaagagaactcaaaatcatgttctaattccatcaacagcaacccctctaggacattcaatgattgtcttgctgaagctatagttgcagaagcagaaagggacaaagaaattgtagtggttcatgcaggaatgggagtcgatccatcacttaagctcttccagtccagatttcctgacagattttttgatgttggcatggcagaacaacatgctattacttttgctgcaggcttatcttgcgggggtttgaaaccgttctgcataattccgtcaacattcttacaaagaggatatgatcaggttatccaagatgtagatctacagagacttcctgtgagatttgccattagtagtgcagggctggcaggatctgaaggtccaattcattctggagtttttgacataacatttatggcatgcttgccaaatatgattgtcatggcaccatcagatgaagatgaacttattgacatggtggctactgctgcttgtgttaacgacaggcctatttgcttccggtatcccagggtagctattatgggaaacaatggtctattacatagtggaatgcctcttgagattgggaagggagagatgctagtagaaggaaaacatgtggctttgcttggctatggtgtgatggttcagaattgcctaaaggcacaatctctgcttgctggcctcggtatccaagtgaccgttgccagtgcaaggttttgcaagccacttgacatcgagcttatccgaaggctatgtcaggagcatgagtttttgataactgtcgaggaaggaaccgttggtggttttggttctcatgtttcacaattcatggcacttgatggtttgcttgatggaagagtaaagtggcgacccattctactaccagacaactacatagagcaagcaaccccaagggaacagctagagattgctggactgaccggccatcacattgcagccacaacattaagtctgttgggacgtcatcgggaggcctttctcttaatgcggtag

SEQ ID NO: 56 - DNA, 2037 bp, Musa acuminata
atggcctctgcttcctctcattgcccgttcagacatatttctttccttcaaagcgaatctaggttccaatctgcggaatctggttactttgggactccgcagttcttgaagaagagcacttctgagttgattatttaccaaaattctgtaactacgtatctaaggaagggttgcagacaggttgctgcactaccagatattggtgatttcttctgggaaaaagatccaactcccattttagacatgacccgtaggcccttcagagcaagtcttgcagtggtggagttaacagtggctttacatcatgtttttcatgctcccatggacaagatactctgggatgatggtgaacagacatatgcacacaagattctgacaggaaggcgctctcttatgcatacacttaagcgaaaagatggtctctcgggtttcacttctcgagcagaaagcgagtacgacgcatttggtgctgggcatggatgcaatagcatatctgctgggcttggcatggcagttgcaagggatattaatggaaagaagaatcgtatagtgacagttataagtaattggacaacgatggctggtcaggtctatgaggcaatgagcaatgctgggtatcttgattctaacatgatagtgattttaaatgatagtaggcactctttacaccctaagcttagtgaaggaccaaaaatgacaatcaatccgatctcaagcactttaagcaagattcaatctagtagatccttccggagattcagggaagctgcaaagggtgtaacgaaaagaatcggtaaaactatgcacgaattggcagctaaagtcgatgagtatacacgtggtatgattggtcctcttggagctactctctttgaagaacttgggctgtactacattggaccagtggatggacacaatattgatgatctaatttgtgtactcaatgaagtggcatcattggattcaactggacccgtattggttcatgtcattacagaagatgaggacttggaaagtattcagaaagagaactcaaaatcatgttctaattccatcaacagcaacccctctaggacattcaatgattgtcttgctgaagctatagttgcagaagcagaaagggacaaagaaattgtagtggttcatgcaggaatgggagtcgatccatcacttaagctcttccagtccagatttcctgacagattttttgatgttggcatggcagaacaacatgctattacttttgctgcaggcttatcttgcgggggtttgaaaccgttctgcataattccgtcaacattcttacaaagaggatatgatcaggttatccaagatgtagatctacagagacttcctgtgagatttgccattagtagtgcagggctggcaggatctgaaggtccaattcattctggagtttttgacataacatttatggcatgcttgccaaatatgattgtcatggcaccatcagatgaagatgaacttattgacatggtggctactgctgcttgtgttaacgacaggcctatttgcttccggtatcccagggtagctattatgggaaacaatggtctattacatagtggaatgcctcttgagattgggaagggagagatgctagtagaaggaaaacatgtggctttgcttggctatggtgtgatggttcagaattgcctaaaggcacaatctctgcttgctggcctcggtatccaagtgaccgttgccagtgcaaggttttgcaagccacttgacatcgagcttatccgaaggctatgtcaggagcatgagtttttgataactgtcgaggaaggaaccgttggtggttttggttctcatgtttcacaattcatggcacttgatggtttgcttgatggaagagtaaagtggcgacccattctactaccagacaactacatagagcaagcaaccccaagggaacagctagagattgctggactgaccggccatcacattgcagccacaacattaagtctgttgggacgtcatcgggaggcctttctcttaatgcggtag

SEQ ID NO: 57 - DNA, 2124 bp, Musa acuminata
atggtggaagcaaggtctctcatggttgcctctgctgctccgttccttaaagctctaagctcgagcgcaaacggcagaagacagctttgcgtgagggcgggtggggcaagcggcgatgggaaggtgatgattacgaaggaaaagagtgggtggaagatcgattactcgggggagaagccagcaacccctctgctggatagcatcaactacccgattcatatgaagaacctctccacgcgggatttggagcagctctcggctgagctcagagcagaaatcgtgttcgctgtggccaagactggcggccacttgagttcgagcttgggagtggtggagttggctgtagctctccatcatgtgttcgatgcccccgaggacaagatcatttgggatgtcggccatcaggcctaccctcataagatattgacggggagaaggtcaaggatgaataccatcaggcagaccgcagggcttgccggatttcccaagagagatgagagcatctatgatgcctttggtgctggccatagttccacaagcatctctgcggggctaggaatggctgttgcaagagatctgctagggaagaagaatcatgttatatctgtcattggcgatggagccatgactgctggccaggcctacgaggccatgaacaatgctggctacttggactccaaccttattatcgtgttgaatgataataagcaagtttcgttaccgactgcaacacttgatggaccagccactcctgttggtgcgctgagtaaggccctcaccaaacttcaatcgagcactaagctgcgcaagctccgtgaagccgctaagaatatcacgaagcagattggtgggcagacacatgacattgctgcaaaggtggatgaatatgctcgtggaatgatgagtgctacagggtattcactgttcgaggagcttggtttgtattatattgggcctgtagatgggcacgatgtggaagacttggttaccatctttgagaaggtgaagtctttgcctgctccgggacctgtccttatccatattgtgacggagaagggcaaggggtatccccccgctgagtctgctgctgacaaaatgcacggtgttgtgaagtttgacccaaaaactgggaagcaattcaaatcaaaatcatccaccctttcgtacactcaatactttgcagagactcttattaaagaagcccaggttgacgacaagatcgtcgctgttcatgctgccatgggtagtgggacagggctgaactattttcagcacaaatttcctgaaagatgctttgatgtgggaattgcagagcagcatgcagtcacctttgcagctggtttggccaccgagggcctcaagcctttctgtgccatctactcatcatttctgcaacgaggatatgatcaggtggttcatgatgtggacttacaaaagatacccgtccggttcgcactggatcgagctggccttgtcggagctgatggacctacccactgtggagcattcgacatcgtgtacatggcatgcttgcccaacatgatcgtaatggccccagccgatgaagccgagctgatgcacatgattgcaacagcggcggcgatcgatgacagacctagctgcttcagattccctagggggaatggagtcggtgtggcccttcctccaaacaacaaaggcacccctcttgagatcgggaagggaagagttctgatggaaggaaacagggtggccatccttggatatggttcaatagtccagacatgcttgaaggctgcagactcactgagatcgcatggaattttccccacagtggctgatgctcggttctgtaaacctctggatgtggagctcataaggagactggcaaatgagcatgagatcctgatcacagtggaggagggctccattggaggtttcggatcgcacgtcactcacttccttggcttgagtggcctgctggataaaaacataaagctgaggtccatggttctaccagatcgatacatcgaccatggatcgccacaggatcaatttgaagtagctggactttcctccagacatattgcagccacagtgctgagtcttttgggcaggcggaaagaggcattgcatctccactga

SEQ ID NO: 58 - DNA, 2124 bp, Musa acuminata
atggaggcttcaggctctctgatggccgctttctccgctccgttcctcgtagctccgaatccaagaaccagccccaagcggcagtttcgtgtcagggcgtgcgggcttggtggtgatgggaagatgatgtttaacaaaggcaagagtgggtggacgattgatttctccggagagaagcctcccaccccgcttctggacaccattaattacccaattcacatgaagaatctctccgtgcaggacttggagcagctcgcagcagagctaagagcagagattgtgttcaccgtgtcgaagactggtgggcatttaagtgcaagcctgggagtcgtggaattgtccgtggctctccatcatgtgttcgatactcccgaggataagatcatatgggatgttggtcatcaggcctacacacataagatcttgaccgggagaaggtcaaggatgcataccgtcaggcaaacctctgggatcgcaggtttccccaggagagatgaaagcatctacgatgcttttggtgctggtcacagctccacaagcatctctgccggactcggcatggccgtcgcccgagatatgctagggaagaagaaccatgtaatctctgtcataggggatggagctatgaccgctggccaggcctacgaagccatgaacaactcaggatacttgaattcgaaccttattgtggtgttgaatgacaacaggcaagtttcattaccaactgcaacccttgatggacctgccactcccgttggtgcactgagtaaagccctcaccagacttcaagcaagtaccaagttccgtaagctccgggaagcagccaagagcatcacaaagcaaattggtggtccaacacatgaggttgctgcgaaggtggatgagttcgccagaggactgataagtgccaatggatcatcattgtttgaggagctgggattatactacatcggtccagtagacgggcacaacttggaagatttggtgaccatcttccaggacgtgaagtccatgcctgctccaggacctgtcctcatccacattgtgacagagaaagggaaagggtatccccccgccgaggctgctccagacaaaatgcacggagtcgtgaagtttgacccgagcaccgggaagcagctgaagccaaagtcacccactcgctcgtacacccagtactttgcggaggctctcatcaaagaggcggaggcggacaacaaggtcgtcgctatccacgcagccatgggtggtgggacgggactgaactacttccagaagaggttccctgaccgatgcttcgacgtgggaattgcagagcagcacgccgtcacgttcgcagctggtctggccaccgagggcctcaagcctttctgtgccatctactcatccttccttcaacgaggatatgatcaggtggtgcatgatgtcgacctccagaagatacctgtccggttcgcgctggatcgagcgggcctcgtcggcgccgatggaccgacgcactgcggagcatttgatatcacgtacatggcttgtttgcccaacatgatcgtgatggccccggcggacgaagccgagctgatgcacatggttgcaactgcggcagccatcgacgaccggcccagctgcttcagatttcccagaggcaacggagtaggtgtggccctccctcccgacaacaagggctcgcctctcgagatcgggaagggcagagttctgatggaaggggacagggccgccatcctgggatacggttccacagttaacacatgcctgaaggctgcagacacgctgagagcccacgcagtcttcgccaccgtggccgacgctcggttctgcaaacctctggacgtcaagctcataaggagcttagtgaaggagcacgatatcttaatcacggtggaggaaggctccatcggaggattcggatcccatgttgctcatttcctgagcttgagtggcctcctcgatggacaactgaagttgagatcgatggttctgccggatcgatacatcgaccatggatcacctcaggatcagattgaagcagcagggctgtcttcaagacatgttgctgcgaccgtgctgtctcttctggggaggcgcaaggaagcgttgctgctgaagtga

SEQ ID NO: 59 - DNA, 2256 bp, Musa acuminata; identical to SEQ ID NO: 6

SEQ ID NO: 60 - DNA, 1908 bp, Musa acuminata
atgaagaatctctcggtccgggagctgaagcaactcgccgacgagctccgctctgatatcatcttcaacgtgtctaggaccggcggtcacctcggttccagcctcggcgtggtcgagctcaccgtcgcgctccactacgtcttcaacgctccgcaggacaagatcctttgggatgtcggccaccagtcgtatcctcacaagatattgacgggaaggagagacaagatggcgacaatgaggcagacgaatggcttgtccgggttcaccaagcggtcggagagcgagtacgactgcttcggtgccggccacagctcgaccagcatatcggcagccctcgggatggcagtcggaagggatctgaaggggcgaaagaacaacgtagtggcagtgattggggacggagccatgaccgcggggcaagcttatgaggccatgaacaatgctggctatctcgactccgacatgattgtgatcttgaatgacaacaagcaggtctctctgcccactgcaactcttgatggccctgttcctccagttggagctctgagcagtgcccttagcagactgcagtcctccaagccactcagggaactgagggaggtcgctaagggagtcacgaagcagatcggtggatccatgcacgaaatagctgccaaagtcgacgaatacgctcgaggaatgatcggtggatcagggtcgaccttgttcgaagagctcggtctctactacatcggtcctgtcgatgggcacaacatagatgacctggtcgccattctcaaggacgtgaagagcaccaagacgacaggccctgttctcatccatgtcgtgaccgagaagggacgagggtatccctacgccgagaaagctgcagacaagtatcatggtgtcgccaaattcgatccagcgacagggaagcaattcaaatcgggctccaagacgcagtcttacacgaactacttcgcggaggcgttgattgccgaggcggaggtggacgaaggcatcgtcgcgatccacgcggccatgggaggaggaacagggctcaactacttccttcgctgctacccgacgaggtgcttcgacgtggggatcgcggagcagcacgcggtcacgtttgcggcagggctcgcctgcgaaggcctcaagccattctgcgcgatctactcgtcgttcctgcagcgggcttacgaccaggtgatacacgacgtggacttgcagaagctgccggtgaggtttgcgatggatcgggcgggactcgtcggagcggacgggccgactcactgcggctccttcgatgtcacctacatggcttgcctaccgaacatggtggtcatggcgccctccgacgaagcggagctgttccacatggtggccaccgcggcggccatcgacgaccggccgtcctgcttccggtaccccaggggcaacggcatcggtgttccgcttccccccggaaacaagggtattccacttgaggtggggaaggggaggatactgaaggaaggggagagggtgactcttctgggatacggaacagcagttcaaagctgcttggccgcggcatcgctgctggaggaacgcggcctaaagatcaccgtcgccgacgcacggttctgcaagccactcgaccggagcctgatccgaaacctggcgaggtcgcacgaggtgctcctcaccgtggaagaaggatccatcggcggtttcggctcccacgtcgtccagttcttggccctcgacggcctcctcgacggcaccctcaagtggcggccggtggttctcccggatcggtacatcgaccatggatcgccgcgcgatcagctggcggaagctggattgacgccgtctcatatcgcagcgactgtgctcaacatcctcggacagacgcgagaggcactcgagatcatgtcttag

SEQ ID NO: 61 - DNA, 2247 bp, Musa acuminata
atggctgcatccacgcttcccttctcttgccatttgcctgctctgctttcctcggatctgcagaaggcttcccccctcctgcctacgcagttgtttgcagggactgatctcccgcaccaccggcatcgtcatgggtttctcacgcctaggagacggtcatgtgtttgcgcctcactatcaggaactggggagtacttctcgcagcggccaccaactccgctgctggacaccgtcaactatcccatccatatgaagaatctctcggtcaaggaactcaaacaacttgcggacgaacttcggtcagatgtcatcttccatgtctctaagacgggaggacatcttggttcgagccttggagtggttgagctaaccgtcgctctacactatgtcttcaatgctcctcaagacaagatactatgggatgttgggcaccagtcgtacccacacaagatactaacagggaggagagacaagatgcctacgttacgacggacgaatggattatctgggttcacaaaacgatcagagagtgactatgatagctttggaactggtcatagttcaaccagcatctcagcagcccttgggatggctgtcggaagggatctgaagggcagaaagaataatgttatagcagtgataggggatggggccatgactgctggacaagcatatgaagctatgaacaatgctgggtatcttgactcggacatgattgtcattctgaatgacaacaagcaggtctctctgcccactgcaagtcttgacgggcctataccaccagttggagctttaagcagtgctctcagtagattacaatctagcagaccattaagagaactgagagaggtcgccaagggagttacgaagcagattggtggatcgatgcatcaaattgcggcaaaagtcgatgaatatgctcgaggaatgattagtggatctggctcaactttgtttgaagagcttggtctctattatattggcccggtggatggccacaacatagatgacctcgtttccatactcaaggaggttaaggacacaaagacaacaggtccagttcttatacatgttgtaacagaaaaaggacggggatatccctatgcagagagagctgctgacaagtatcatggtgttaccaaatttgatccggccactgggaaacaattgaagtcgatctctcagactcaatcttataccaattattttgctgaagctttgatagctgaggcagaggtagacaaagatatagtcgcaattcatgcagccatgggaggtggaaccggccttaactacttccttcgtcgatttccaacaagatgttttgatgtcggtatagccgagcagcatgctgttacatttgcagctggtctagcctgcgaaggcctcaagccattctgtgcaatctactcatctttcttgcaacgggcttacgatcaggcaagccattgccctcatttctccattctgagctttgacaaagttaagccaactagatcgagcaatgatgaatttgagcttttaatgcaggtgatacatgatgtggacttgcagaaacttcctgtaagatttgctatggaccgagcggggcttgtcggagctgatgggccaactcattgtggtgcatttgatgtcacatacatggcatgtctgcctaatatgattgtcatggctccttccgatgaagctgaactgtttcacatggttgccactgcagcagccatcaatgaccggccatcctgcttccgatatccaagaggaaatggcattggcgttcccctgccccaaggaaacaaaggtgttccgcttgagatcggcaaaggcaggatattgattgagggtgagagggtggctcttcttggatatggaacagcagttcagagctgtgtggctgcagcttccctcctggaacaacgtggtctaagggtcacagtggctgatgcacgattctgcaagccgctggatcatgctttgattcggaacttatctaaatctcaccaagtgctgattacagttgaagaaggatccatcggagggtttggctctcatgtcgcccagttcatggcacttaatggtcttcttgatggcacgataaagtggagaccgctggttcttcctgatcgttacatcgagcatggatcacccaatgatcagctggcagaagctggtttgacaccgtctcatgttgcagccacagtgctcaacatccttggacaaactagagaggcacttgaaatcatgtcatag

SEQ ID NO: 62 - DNA, 1227 bp, Musa acuminata
atgataagcactgatgggtctttgttattcgaagagctcggtctctattatattggacctgtagatgggcacaatgtagaagacttggttaccatctttgagaaggtgaagtctttgcctgctccaggacctgtccttatccatattgtgacagagaaaggaaaggggtatccccctgctgaggcggctgctgacaaaatgcatggtgttgtgaagttcgacccaagaactgggaaacaattcaagtcaacatcatcgaccctttcatacactcagtactttgccgaatctctcattaaagaagcagaggccgacgacaagattgtggccattcatgctgccatgggaagtgggacggggctgaacttgtttcaacacaagtttcctcaaagatgctttgatgtggggattgcagagcagcatgcagtcacctttgcagccggtctggccaccgaaggcctcaagcctttctgtgccatctattcctcgtttctgcaacgaggatatgatcaggtggttcatgatgtggatttacagaagatacctgtccgtttcgctctggatcgagctggtcttgtcggagctgatggacctacacactgtggagcatttgacatcacgtacatggcatgtttgcccaacatgattgtaatggctccagctgatgaagctgagctagtgcacatggtcgcaacagcagcagcaatcgacgacagacctagctgcttcagattcccaaggggcaatggagttggtgtgatgcttcctccgggcaacaaaggcaccccttttgagattgggaagggaagggttctgatggaaggaaacagggtggccattcttggatatggttcaatagtacagacatgcttgaaggctgcagacccactgagagcccgtggagtttttgccaccgtagctgatgctcgtttctgtaagcctctggatgtggggctcataagaaggctggtaaatgagcatgagatcttgatcacagtggaggaaggctccattggaggtttcgcatcgcatgtcactcacttcttgagcttgagtggcctcctggatggccgcatgaagctgaggccaatggttctaccagaccgatacatcgaccatggatcacctcaggatcagattgaagcagctggactttcttcaggacatattgtaagcacagtgctgaatctgttaggcaggcagaaggaagcattatacctccattga

SEQ ID NO: 63 - DNA, 2133 bp, Musa acuminata; identical to SEQ ID NO: 55

SEQ ID NO: 64 - DNA, 2076 bp, Musa acuminata
atggtggaagcaaggtctctcatggttgcctctgctgctccgttccttaaagctctaagctcgagcgcaaacggcagaagacagctttgcgtgagggcgggtggggcaagcggcgatgggaaggtgatgattacgaaggaaaagagtgggtggaagatcgattactcgggggagaagccagcaacccctctgctggatagcatcaactacccgattcatatgaagaacctctccacgcgggatttggagcagctctcggctgagctcagagcagaaatcgtgttcgctgtggccaagactggcggccacttgagttcgagcttgggagtggtggagttggctgtagctctccatcatgtgttcgatgcccccgaggacaagatcatttgggatgtcggccatcaggcctaccctcataagatattgacggggagaaggtcaaggatgaataccatcaggcagaccgcagggcttgccggatttcccaagagagatgagagcatctatgatgcctttggtgctggccatagttccacaagcatctctgcggggctaggaatggctgttgcaagagatctgctagggaagaagaatcatgttatatctgtcattggcgatggagccatgactgctggccaggcctacgaggccatgaacaatgctggctacttggactccaaccttattatcgtgttgaatgataataagcaagtttcgttaccgactgcaacacttgatggaccagccactcctgttggtgcgctgagtaaggccctcaccaaacttcaatcgagcactaagctgcgcaagctccgtgaagccgctaagaatatcacgaagcagattggtgggcagacacatgacattgctgcaaaggtggatgaatatgctcgtggaatgatgagtgctacagggtattcactgttcgaggagcttggtttgtattatattgggcctgtagatgggcacgatgtggaagacttggttaccatctttgagaaggtgaagtctttgcctgctccgggacctgtccttatccatattgtgacggagaagggcaaggggtatccccccgctgagtctgctgctgacaaaatgcacggtgttgtgaagtttgacccaaaaactgggaagcaattcaaatcaaaatcatccaccctttcgtacactcaatactttgcagagactcttattaaagaagcccaggttgacgacaagatcgtcgctgttcatgctgccatgggtagtgggacagggctgaactattttcagcacaaatttcctgaaagatgctttgatgtgggaattgcagagcagcatgcagtcacctttgcagctggtttggccaccgagggcctcaagcctttctgtgccatctactcatcatttctgcaacgaggatatgatcaggtggttcatgatgtggacttacaaaagatacccgtccggttcgcactggatcgagctggccttgtcggagctgatggacctacccactgtggagcattcgacatcgtgtacatggcatgcttgcccaacatgatcgtaatggccccagccgatgaagccgagctgatgcacatgattgcaacagcggcggcgatcgatgacagacctagctgcttcagattccctagggggaatggagtcggtgtggcccttcctccaaacaacaaaggcacccctcttgagatcgggaagggaagagttctgatggaaggaaacagggtggccatccttggatatggttcaatagtccagacatgcttgaaggctgcagactcactgagatcgcatggaattttccccacagtggctgatgctcggttctgtaaacctctggatgtggagctcataaggagactggcaaatgagcatgagatcctgatcacagtggaggagggctccattggaggtttcggatcgcacctgaggtccatggttctaccagatcgatacatcgaccatggatcgccacaggatcaatttgaagtagctggactttcctccagacatattgcagccacagtgctgagtcttttgggcaggcggaaagaggcattgcatctccactga

SEQ ID NO: 65 - DNA, 2124 bp, Musa acuminata; identical to SEQ ID NO: 58

SEQ ID NO: 66 - DNA, 23 bp, Artificial sequence
  single guide RNA (sgRNA) nucleic acid sequence
gaggctagagatgtcctgggtgg

SEQ ID NO: 67 - DNA, 23 bp, Artificial sequence
  single guide RNA (sgRNA) nucleic acid sequence
catctttctgcaatggtccacgg

SEQ ID NO: 68 - DNA, 23 bp, Artificial sequence
  single guide RNA (sgRNA) nucleic acid sequence
gtctctcccatgaagttaagtgg

SEQ ID NO: 69 - DNA, 23 bp, Artificial sequence
  single guide RNA (sgRNA) nucleic acid sequence
tttctgcactaagcctgaccagg

SEQ ID NO: 70 - DNA, 23 bp, Artificial sequence
  single guide RNA (sgRNA) nucleic acid sequence
tttggaggtggtgattctatggg

SEQ ID NO: 71 - DNA, 23 bp, Artificial sequence
  single guide RNA (sgRNA) nucleic acid sequence
tgaaaatgccgtcaactatttgg

SEQ ID NO: 72 - DNA, 23 bp, Artificial sequence
  single guide RNA (sgRNA) nucleic acid sequence
ccgtacttctcctcatccaaata

SEQ ID NO: 73 - DNA, 20 bp, Artificial sequence
  single guide RNA (sgRNA) nucleic acid sequence
gggcgaggagctgttcaccg

SEQ ID NO: 74 - DNA, 20 bp, Artificial sequence
  single guide RNA (sgRNA) nucleic acid sequence
ggccacaagttcagcgtgtc